SEQUENCE LISTING (32 sequences)

SEQ ID NO: 1 - protein, 1273 aa, Unknown
  Coronaviridae
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 29905 bp, Unknown
  S1 (Subunit 1 of Spike protein) of Coronaviridae
  misc_feature (694)..(694): n is a, c, g, or t
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatanttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 3 - protein, 542 aa, Artificial Sequence
  S1 (Subunit 1 of Spike protein)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFN

SEQ ID NO: 4 - protein, 731 aa, Artificial Sequence
  S2 (Subunit 2 of Spike protein and S1/S2 cleavage region)
FNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1273 aa, Artificial Sequence
  D614G spike protein variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 215 aa, Homo sapiens
MVLLTMIARVADGLPLAASMQEDEQSGRDLQQYQSQAKQLFRKLNEQSPTRCTLEAGAMTFHYIIEQGVCYLVLCEAAFPKKLAFAYLEDLHSEFDEQHGKKVPTVSRPYSFIEFDTFIQKTKKLYIDSRARRNLGSINTELQDVQRIMVANIEEVLQRGEALSALDSKANNLSSLSKKYRQDAKYLNMRSTYAKLAAVAVFFIMLIVYVRFWWL

SEQ ID NO: 7 - DNA, 648 bp, Homo sapiens
atggtgttgctaacaatgatcgcccgagtggcggacgggctcccgctggccgcctcgatgcaggaggacgaacagtctggccgggaccttcaacaatatcagagtcaggctaagcaactctttcgaaagttgaatgaacagtcccctaccagatgtaccttggaagcaggagccatgacttttcactacattattgagcagggggtgtgttatttggttttatgtgaagctgccttccctaagaagttggcttttgcctacctagaagatttgcactcagaatttgatgaacagcatggaaagaaggtgcccactgtgtcccgaccctattcctttattgaatttgatactttcattcagaaaaccaagaagctctacattgacagtcgtgctcgaagaaatctaggctccatcaacactgaattgcaagatgtgcagaggatcatggtggccaatattgaagaagtgttacaacgaggagaagcactctcagcattggattcaaaggctaacaatttgtccagtctgtccaagaaataccgccaggatgcgaagtacttgaacatgcgttccacttatgccaaacttgcagcagtagctgtatttttcatcatgttaatagtgtatgtccgattctggtggctgtga

SEQ ID NO: 8 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - DNA, 762 bp, Homo sapiens
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 10 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 11 - DNA, 378 bp, Homo sapiens
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 12 - protein, 261 aa, Homo sapiens [sequence omitted]

SEQ ID NO: 13 - DNA, 841 bp, Homo sapiens
tttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttgtcttactcaaactctgaacagtgtcaccttgcaggctgtggtggagctgacgctgggagtc

SEQ ID NO: 14 - protein, 559 aa, Homo sapiens
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 15 - DNA, 1680 bp, Homo sapiens
atgtcggccaccagcgtggacacccagagaacaaaaggacaagataataaagtacaaaatggttcgttacatcagaaggatacagttcatgacaatgactttgagccctaccttactggacagtcaaatcagagtaacagttacccctcaatgagcgacccctacctgtccagctattacccgccgtccattggatttccttactccctcaatgaggctccgtggtctactgcaggggaccctccgattccatacctcaccacctacggacagctcagtaacggagaccatcattttatgcacgatgctgtttttgggcagcctgggggcctggggaacaacatctatcagcacaggttcaattttttccctgaaaaccctgcgttctcagcatgggggacaagtgggtctcaaggtcagcagacccagagctccgcgtatgggagcagctacacctaccccccgagctccctgggtggcacggtggttgatgggcagccaggctttcacagcgacaccctcagcaaggcccccgggatgaacagcctggagcagggcatggttggcctgaagattggggacgtcagctcctccgccgtcaagacggtgggctctgtcgtcagcagcgtggcactgactggtgtcctttctggcaacggtgggacaaatgtgaacatgccagtttcaaagccgacctcgtgggctgccattgccagcaagcctgcaaaaccacagcctaaaatgaaaacaaagagcgggcctgtcatggggggtgggctgccccctccacccataaagcataacatggacattggcacctgggataacaaggggcctgtgccgaaggccccagtcccccagcaggcaccctctccacaggctgccccacagccccagcaggtggctcagcctctcccagcacagcccccagctttggctcaaccgcagtatcagagccctcagcagccaccccagacccgctgggttgccccacgcaacagaaacgcggcgtttgggcagagcggaggggctggcagcgatagcaactctcctggaaacgtccagcctaattctgcccccagcgtcgaatcccaccccgtccttgaaaaactgaaggctgctcacagctacaacccgaaagagtttgagtggaatctgaaaagcgggcgtgtgttcatcatcaagagctactctgaggacgacatccaccgctccattaagtactccatctggtgtagcacagagcacggcaacaagcgcctggacagcgccttccgctgcatgagcagcaaggggcccgtctacctgctcttcagcgtcaatgggagtgggcatttttgtggggtggccgagatgaagtcccccgtggactacggcaccagtgccggggtctggtctcaggacaagtggaaggggaagtttgatgtccagtggatttttgttaaggatgtacccaataaccagctccggcacatcaggctggagaataacgacaacaaaccggtcacaaactcccgggacacccaggaggtgcccttagaaaaagccaagcaagtgctgaaaattatcagttcctacaagcacacaacctccatcttcgacgactttgctcactacgagaagcgccaggaggaggaggaggtggtgcgcaaggaacggcagagtcgaaacaaacaatga

SEQ ID NO: 16 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 17 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 18 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 19 - DNA, 822 bp, Homo sapiens
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctga

SEQ ID NO: 20 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 21 - DNA, 834 bp, Homo sapiens
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 22 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 23 - DNA, 582 bp, Homo sapiens
atgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctga

SEQ ID NO: 24 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASRVDAKAILSARLSKSRRLENLIAQLPGEKKNGLFGNLIALLLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDKEMIEERLKKYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWKQLLNAKLITQRKFDNLTKAERGGLSELDKAGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 25 - protein, 994 aa, Staphylococcus agnetis
MNNYILGLDIGITSVGYGIVDSDTREIKDAGVRLFPEANVDNNEGRRSKRGARRLKRRRIHRLDRVKHLLAEYNLLDLTNIPKSTNPYQIRVKGLNEKLSKDELVIALLHIAKRRGIHNVNVMMDDNDSGNELSTKDQLKKNAKALSDKYVCELQLERFEQDYKVRGEKNRFKTEDFVREARKLLETQSKFFEIDQTFIMRYIDLVETRREYFEGPGKGSPFGWEGNIKKWFEQMMGHCTYFPEELRSVKYAYSAELFNALNDLNNLVITRDEEAKLNYGEKFQIIENVFKQKKTPNLKQIAKEIGVSETDIKGYRVNKSGKPEFTQFKLYHDLKNIFEDSKYLNDVQLMDNIAEIITIYQDPESIIKELNQLPELLSEKEKEKISALSGYAGTHRLSLKCINLLLDDLWESSLNQMELFTKLNLKPKKIDLSQQHKIPIKLVDDFILSPVVKRAFIQSIQVVNAIIDKYGLPEDIIIELARENNSDDRRKFLNQLQKQNAETRKQVEKVLREYGNDNAKRIVQKIKLHNMQEGKCLYSLKDIPLEDLLKNPNHYEVDHIIPRSVAFDNSMHNKVLVRAEENSKKGNRTPYQYLNSSESSLSYNEFKQHILNLSKTKDRITKKKREYLLEERDINKYDVQKEFINRNLVDTRYATRELTSLLKAYFSANNLDVKVKTINGSFTNYLRKVWKFDKDRNKGYKHHAEDALIIANADFLFKHNKKLRNINKVLDAPSKEVDKKRVTVQSEDEYNQMFEDTQKAQAIKKFEIRKFSHRVDKKPNRQLIKDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLKIYSYRFDIYQYGNNYKMITISYIDLEQKSNYYYISREKYEQKKKDKQIDDSYKFIGSFYKNDIINYNGEMYRVIGVNDSEKIKFSLI

SEQ ID NO: 26 - protein, 1110 aa, Artificial Sequence
  Synthetic construct derived from Staphylococcus aureus Cas9
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTREQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEERQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSYPYDVPDYASGFANELGPRLMGK

SEQ ID NO: 27 - protein, 1235 aa, Candidatus Methanomethylophilus alvus
MHTGGLLSMDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 28 - protein, 1227 aa, Candidatus Methanomethylophilus alvus
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 29 - protein, 1238 aa, Candidatus Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 30 - protein, 1273 aa, Artificial Sequence
  COVID19 variant
MFVFLVLLPLVSSQCVNFTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHISYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSKNLDSKVGGNYNYRFRLFRKSNLKPFERDISTEIYQAGNTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTPTNSHRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - DNA, 3819 bp, Artificial Sequence
  COVID19 variant DNA
atgtttgtgtttctggtgctgctgccgctggtgagcagccagtgcgtgaactttaccacccgcacccagctgccgccggcgtataccaacagctttacccgcggcgtgtattatccggataaagtgtttcgcagcagcgtgctgcatagcacccaggatctgtttctgccgttttttagcaacgtgacctggtttcatgcgattcatgtgagcggcaccaacggcaccaaacgctttgataacccggtgctgccgtttaacgatggcgtgtattttgcgagcaccgaaaaaagcaacattattcgcggctggatttttggcaccaccctggatagcaaaacccagagcctgctgattgtgaacaacgcgaccaacgtggtgattaaagtgtgcgaatttcagttttgcaacgatccgtttctgggcgtgtattatcataaaaacaacaaaagctggatggaaagcgaatttcgcgtgtatagcagcgcgaacaactgcacctttgaatatgtgagccagccgtttctgatggatctggaaggcaaacagggcaactttaaaaacctgcgcgaatttgtgtttaaaaacattgatggctattttaaaatttatagcaaacataccccgattaacctggtgcgcgatctgccgcagggctttagcgcgctggaaccgctggtggatctgccgattggcattaacattacccgctttcagaccctgctggcgctgcatattagctatctgaccccgggcggcagcagcagcggctggaccgcgggcgcggcggcgtattatgtgggctatctgcagccgcgcacctttctgctgaaatataacgaaaacggcaccattaccgatgcggtggattgcgcgctggatccgctgagcgaaaccaaatgcaccctgaaaagctttaccgtggaaaaaggcatttatcagaccagcaactttcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaacattgcggattataactataaactgccggatgattttaccggctgcgtgattgcgtggaacagcaaaaacctggatagcaaagtgggcggcaactataactatcgctttcgcctgtttcgcaaaagcaacctgaaaccgtttgaacgcgatattagcaccgaaatttatcaggcgggcaacaccccgtgcaacggcgtgaaaggctttaactgctattttccgctgcagagctatggctttcagccgacctatggcgtgggctatcagccgtatcgcgtggtggtgctgagctttgaactgctgcatgcgccggcgaccgtgtgcggcccgaaaaaaagcaccaacctggtgaaaaacaaatgcgtgaactttaactttaacggcctgaccggcaccggcgtgctgaccgaaagcaacaaaaaatttctgccgtttcagcagtttggccgcgatattgcggataccaccgatgcggtgcgcgatccgcagaccctggaaattctggatattaccccgtgcagctttggcggcgtgagcgtgattaccccgggcaccaacaccagcaaccaggtggcggtgctgtatcagggcgtgaactgcaccgaagtgccggtggcgattcatgcggatcagctgaccccgacctggcgcgtgtatagcaccggcagcaacgtgtttcagacccgcgcgggctgcctgattggcgcggaacatgtgaacaacagctatgaatgcgatattccgattggcgcgggcatttgcgcgagctatcagaccccgaccaacagccatcgccgcgcgcgcagcgtggcgagccagagcattattgcgtataccatgagcctgggcgtggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatgcagatggcgtatcgctttaacggcattggcgtgacccagaacgtgctgtatgaaaaccagaaactgattgcgaaccagtttaacagcgcgattggcaaaattcaggatagcctgagcagcaccgcgagcgcgctgggcaaactgcaggatgtggtgaaccagaacgcgcaggcgctgaacaccctggtgaaacagctgagcagcaactttggcgcgattagcagcgtgctgaacgatattctgagccgcctggataaagtggaagcggaagtgcagattgatcgcctgattaccggccgcctgcagagcctgcagacctatgtgacccagcagctgattcgcgcggcggaaattcgcgcgagcgcgaacctggcggcgaccaaaatgagcgaatgcgtgctgggccagagcaaacgcgtggatttttgcggcaaaggctatcatctgatgagctttccgcagagcgcgccgcatggcgtggtgtttctgcatgtgacctatgtgccggcgcaggaaaaaaactttaccaccgcgccggcgatttgccatgatggcaaagcgcattttccgcgcgaaggcgtgtttgtgagcaacggcacccattggtttgtgacccagcgcaacttttatgaaccgcagattattaccaccgataacacctttgtgagcggcaactgcgatgtggtgattggcattgtgaacaacaccgtgtatgatccgctgcagccggaactggatagctttaaagaagaactggataaatattttaaaaaccataccagcccggatgtggatctgggcgatattagcggcattaacgcgagcgtggtgaacattcagaaagaaattgatcgcctgaacgaagtggcgaaaaacctgaacgaaagcctgattgatctgcaggaactgggcaaatatgaacagtatattaaatggccgtggtatatttggctgggctttattgcgggcctgattgcgattgtgatggtgaccattatgctgtgctgcatgaccagctgctgcagctgcctgaaaggctgctgcagctgcggcagctgctgcaaatttgatgaagatgatagcgaaccggtgctgaaaggcgtgaaactgcattatacc

SEQ ID NO: 32 - RNA, 3819 nt, Artificial Sequence
  COVID19 variant mRNA
auguuuguguuucuggugcugcugccgcuggugagcagccagugcgugaacuuuaccacccgcacccagcugccgccggcguauaccaacagcuuuacccgcggcguguauuauccggauaaaguguuucgcagcagcgugcugcauagcacccaggaucuguuucugccguuuuuuagcaacgugaccugguuucaugcgauucaugugagcggcaccaacggcaccaaacgcuuugauaacccggugcugccguuuaacgauggcguguauuuugcgagcaccgaaaaaagcaacauuauucgcggcuggauuuuuggcaccacccuggauagcaaaacccagagccugcugauugugaacaacgcgaccaacguggugauuaaagugugcgaauuucaguuuugcaacgauccguuucugggcguguauuaucauaaaaacaacaaaagcuggauggaaagcgaauuucgcguguauagcagcgcgaacaacugcaccuuugaauaugugagccagccguuucugauggaucuggaaggcaaacagggcaacuuuaaaaaccugcgcgaauuuguguuuaaaaacauugauggcuauuuuaaaauuuauagcaaacauaccccgauuaaccuggugcgcgaucugccgcagggcuuuagcgcgcuggaaccgcugguggaucugccgauuggcauuaacauuacccgcuuucagacccugcuggcgcugcauauuagcuaucugaccccgggcggcagcagcagcggcuggaccgcgggcgcggcggcguauuaugugggcuaucugcagccgcgcaccuuucugcugaaauauaacgaaaacggcaccauuaccgaugcgguggauugcgcgcuggauccgcugagcgaaaccaaaugcacccugaaaagcuuuaccguggaaaaaggcauuuaucagaccagcaacuuucgcgugcagccgaccgaaagcauugugcgcuuuccgaacauuaccaaccugugcccguuuggcgaaguguuuaacgcgacccgcuuugcgagcguguaugcguggaaccgcaaacgcauuagcaacugcguggcggauuauagcgugcuguauaacagcgcgagcuuuagcaccuuuaaaugcuauggcgugagcccgaccaaacugaacgaucugugcuuuaccaacguguaugcggauagcuuugugauucgcggcgaugaagugcgccagauugcgccgggccagaccggcaacauugcggauuauaacuauaaacugccggaugauuuuaccggcugcgugauugcguggaacagcaaaaaccuggauagcaaagugggcggcaacuauaacuaucgcuuucgccuguuucgcaaaagcaaccugaaaccguuugaacgcgauauuagcaccgaaauuuaucaggcgggcaacaccccgugcaacggcgugaaaggcuuuaacugcuauuuuccgcugcagagcuauggcuuucagccgaccuauggcgugggcuaucagccguaucgcgugguggugcugagcuuugaacugcugcaugcgccggcgaccgugugcggcccgaaaaaaagcaccaaccuggugaaaaacaaaugcgugaacuuuaacuuuaacggccugaccggcaccggcgugcugaccgaaagcaacaaaaaauuucugccguuucagcaguuuggccgcgauauugcggauaccaccgaugcggugcgcgauccgcagacccuggaaauucuggauauuaccccgugcagcuuuggcggcgugagcgugauuaccccgggcaccaacaccagcaaccagguggcggugcuguaucagggcgugaacugcaccgaagugccgguggcgauucaugcggaucagcugaccccgaccuggcgcguguauagcaccggcagcaacguguuucagacccgcgcgggcugccugauuggcgcggaacaugugaacaacagcuaugaaugcgauauuccgauuggcgcgggcauuugcgcgagcuaucagaccccgaccaacagccaucgccgcgcgcgcagcguggcgagccagagcauuauugcguauaccaugagccugggcguggaaaacagcguggcguauagcaacaacagcauugcgauuccgaccaacuuuaccauuagcgugaccaccgaaauucugccggugagcaugaccaaaaccagcguggauugcaccauguauauuugcggcgauagcaccgaaugcagcaaccugcugcugcaguauggcagcuuuugcacccagcugaaccgcgcgcugaccggcauugcgguggaacaggauaaaaacacccaggaaguguuugcgcaggugaaacagauuuauaaaaccccgccgauuaaagauuuuggcggcuuuaacuuuagccagauucugccggauccgagcaaaccgagcaaacgcagcuuuauugaagaucugcuguuuaacaaagugacccuggcggaugcgggcuuuauuaaacaguauggcgauugccugggcgauauugcggcgcgcgaucugauuugcgcgcagaaauuuaacggccugaccgugcugccgccgcugcugaccgaugaaaugauugcgcaguauaccagcgcgcugcuggcgggcaccauuaccagcggcuggaccuuuggcgcgggcgcggcgcugcagauuccguuugcgaugcagauggcguaucgcuuuaacggcauuggcgugacccagaacgugcuguaugaaaaccagaaacugauugcgaaccaguuuaacagcgcgauuggcaaaauucaggauagccugagcagcaccgcgagcgcgcugggcaaacugcaggauguggugaaccagaacgcgcaggcgcugaacacccuggugaaacagcugagcagcaacuuuggcgcgauuagcagcgugcugaacgauauucugagccgccuggauaaaguggaagcggaagugcagauugaucgccugauuaccggccgccugcagagccugcagaccuaugugacccagcagcugauucgcgcggcggaaauucgcgcgagcgcgaaccuggcggcgaccaaaaugagcgaaugcgugcugggccagagcaaacgcguggauuuuugcggcaaaggcuaucaucugaugagcuuuccgcagagcgcgccgcauggcgugguguuucugcaugugaccuaugugccggcgcaggaaaaaaacuuuaccaccgcgccggcgauuugccaugauggcaaagcgcauuuuccgcgcgaaggcguguuugugagcaacggcacccauugguuugugacccagcgcaacuuuuaugaaccgcagauuauuaccaccgauaacaccuuugugagcggcaacugcgauguggugauuggcauugugaacaacaccguguaugauccgcugcagccggaacuggauagcuuuaaagaagaacuggauaaauauuuuaaaaaccauaccagcccggauguggaucugggcgauauuagcggcauuaacgcgagcguggugaacauucagaaagaaauugaucgccugaacgaaguggcgaaaaaccugaacgaaagccugauugaucugcaggaacugggcaaauaugaacaguauauuaaauggccgugguauauuuggcugggcuuuauugcgggccugauugcgauugugauggugaccauuaugcugugcugcaugaccagcugcugcagcugccugaaaggcugcugcagcugcggcagcugcugcaaauuugaugaagaugauagcgaaccggugcugaaaggcgugaaacugcauuauacc

(19) United States
(12) Patent Application Publication
(10) Pub. No.: US 2021/0290757 A1
(43) Pub. Date: Sep. 23, 2021

(54) ENGINEERING OF DENDRITIC CELLS FOR GENERATION OF VACCINES AGAINST SARS-COV-2

(71) Applicant: Avectas Limited, Co. Kildare (IE)

(72) Inventors: Shirley O'Dea, Maynooth (IE); Michael Maguire, Dublin (IE)

(21) Appl. No.: 17/210,409

(22) Filed: Mar. 23, 2021

Related U.S. Application Data
(60) Provisional application No. 62/993,461, filed on Mar. 23, 2020.

(51) Int. Cl. A61K 39/215

(52) U.S. Cl. CPC A61K 39/215; A61K 2039/5154

(57) ABSTRACT

The invention relates to methods of engineering cells (e.g., dendritic cells (DCs)) for vaccinations (e.g., COVID-19) using ethanol-based transient cell membrane permeabilization. Related methods, compositions, apparatus, systems, and articles as described and/or illustrated herein.

### RELATED APPLICATIONS

This application claims the benefit of priority under 35 U.S.C. § 119(e) to U.S. Provisional Application No. 62/993,461, filed Mar. 23, 2020, the entire contents of which is incorporated herein by reference in its entirety.

### INCORPORATION BY REFERENCE OF SEQUENCE LISTING

The contents of the sequence listing text file named “048831-524001US_Sequence_Listing_ST25.txt”, which was created on Jun. 4, 2021 and is 188,046 bytes in size, is hereby incorporated by reference in its entirety.

### FIELD OF THE INVENTION

The invention relates to engineering dendritic cells (DCs) for vaccinations.

### BACKGROUND OF THE INVENTION

Severe acute respiratory syndrome (SARS) is a viral respiratory illness caused by a coronavirus called SARS-associated coronavirus (SARS-CoV). SARS-CoV-2 is a new coronavirus that is responsible for the 2020 COVID-19 global pandemic. Although vaccines are currently available for COVID-19, variants have emerged and continue to emerge in the population. Some variants are more infectious and/or more deadly than the originally-identified virus. Thus, improved vaccines are urgently required. A vaccine is a biological preparation that provides active acquired immunity to a particular infectious disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. Thus new vaccines and treatments are urgently needed.

### SUMMARY OF THE INVENTION

The invention provides an improved vaccine against coronavirus infection and disease. The invention also provides a solution to the problem of efficiently delivering payload/cargo (e.g., coronavirus antigens, conventional mRNA molecules, synthetic mRNAs, DNA-encoding antigens or SARS-CoV-2 proteins or peptides) compounds and compositions into cells, e.g., dendritic cells (DCs), which play an important role in immunity against infectious agents such as coronavirus COVID-19. As described herein, the SOLUPORE™ system is used to engineer DCs such that the DCs (i) present coronavirus antigens and (ii) have enhanced functionality, e.g., the ability to present antigen to immune effector cells to elicit a productive and protective immune response based on the delivered antigen(s). The SOLUPORE™ system can refer to technology related to, associated with, and including an approach to delivering payload/cargo and compositions into cells using alcohol and a spray delivery means.

DC vaccines are generated using the SOLUPORE™ system to deliver mRNA encoding for SARS-CoV-2 antigens to autologous dendritic cells ex vivo. For example, blood, e.g., peripheral blood is taken from a subject, optionally processed to purify or enrich for dendritic cells, and then contacting the autologous dendritic cells with mRNA encoding for SARS-CoV-2 antigens after which the modified dendritic cells are then infused or injected back into the same subject from which they came. In other examples, DC vaccines are generated using the SOLUPORE™ system to deliver mRNA encoding for SARS-CoV-2 antigens to allogeneic cells ex vivo. Exemplary allogeneic cells are cell lines, e.g., immortalized cells. For example, the cells include DCOne cells (from DCPrime) or MUTZ-3 cells [available from DSMZ, German Collection of Microrganisms and Cell Cultures (https://www.dsmz.de/collection/catalogue/details/culture/ACC-295)].

Moreover, in addition to conventional mRNA molecules, synthetic mRNAs that are expressed more rapidly are used in order to achieve more rapid in vivo responses (see, e.g., U.S. Pat. No. 9,657,282 Factor Bio, incorporated herein by reference in its entirety. In particular, see col. 3: 1-16; col. 10: 48-col. 15:49 and col. 14: 14-48 of U.S. Pat. No. 9,657,282. Synthetic mRNAs can be customized to encode the a protein antigen or composite protein antigen, e.g., w a COVID-19 spike protein that includes 1 or more, e.g., 2, 3, 4, 5, 6, 7, 8, 9, 10 or more point mutations that are associated with COVID virus variants such as more infectious or deadly existing variants or projected variants such as those with predicted dangerous point mutations that lead to increased infectivity or severity of disease.

In embodiments, DNA-encoding antigens or SARS-CoV-2 proteins or peptides are delivered to autologous or allogeneic DCs using the SOLUPORE™ technology. As used herein, the term “autologous” refers to, or involving tissues or cells that are from one's own body or bodily tissue/fluid sample. The term “allogenic” refers to tissues or cells that are genetically dissimilar and hence immunologically incompatible, although from individuals of the same species.

In embodiments, ‘TriMix’ mRNAs are delivered in order to enhance DC functionality. The TriMix approach involves mRNA transfection-based delivery of antigens alongside a combination of cluster of differentiation 40 ligand (CD40L), constitutively active toll receptor 4 (caTLR4), and cluster of differentiation 70 (CD70) encoding mRNAs.

DCs transfected with TriMix demonstrate an enhanced T cell activation potential. Vaccination with autologous TriMix-DCs has been shown to be safe and capable of antigen-specific immune response activation.

In embodiments, DCs are engineered to express proteins that enhance DC functionality. For example, the Soluble NSF attachment proteins (SNAP) Receptor protein (SNARE) protein includes vesicle tracking protein SEC22b (SEC22B) reduces antigen degradation by DCs. Delivery of SEC22b-encoding DNA or mRNA enhances DC functionality. The human SEC22B amino acid sequence is provided below (SEQ ID NO: 6)

MVLLTMIARVADGLPLAASMQEDEQSGRDLQQYQSQAKQLFRKLNEQSPT
RCTLEAGAMTFHYIIEQGVCYLVLCEAAFPKKLAFAYLEDLHSEFDEQHG
KKVPTVSRPYSFIEFDTFIQKTKKLYIDSRARRNLGSINTELQDVQRIMV
ANIEEVLQRGEALSALDSKANNLSSLSKKYRQDAKYLNMRSTYAKLAAVA
VFFIMLIVYVRFWWL

The human SEC22B nucleic acid sequence is provided below (SEQ ID NO: 7)

ATGGTGTTGCTAACAATGATCGCCCGAGTGGCGGACGGGCTCCCGCTGGC
CGCCTCGATGCAGGAGGACGAACAGTCTGGCCGGGACCTTCAACAATATC
AGAGTCAGGCTAAGCAACTCTTTCGAAAGTTGAATGAACAGTCCCCTACC
AGATGTACCTTGGAAGCAGGAGCCATGACTTTTCACTACATTATTGAGCA
GGGGGTGTGTTATTTGGTTTTATGTGAAGCTGCCTTCCCTAAGAAGTTGG
CTTTTGCCTACCTAGAAGATTTGCACTCAGAATTTGATGAACAGCATGGA
AAGAAGGTGCCCACTGTGTCCCGACCCTATTCCTTTATTGAATTTGATAC
TTTCATTCAGAAAACCAAGAAGCTCTACATTGACAGTCGTGCTCGAAGAA
ATCTAGGCTCCATCAACACTGAATTGCAAGATGTGCAGAGGATCATGGTG
GCCAATATTGAAGAAGTGTTACAACGAGGAGAAGCACTCTCAGCATTGGA
TTCAAAGGCTAACAATTTGTCCAGTCTGTCCAAGAAATACCGCCAGGATG
CGAAGTACTTGAACATGCGTTCCACTTATGCCAAACTTGCAGCAGTAGCT
GTATTTTTCATCATGTTAATAGTGTATGTCCGATTCTGGTGGCTGTGA

Another example is expression of interleukin 12 (IL-12) or Chemokine (C-X-C motif) ligand 9 (CXCL9) to enhance T cell activation by DCs. In still another example, induction of CD40L expression via mRNA is well established as a maturation tool in some DC vaccines.

The human amino acid sequence for IL-12 is provided below (SEQ ID NO: 8)

MWPPGSASQPPPSPAAATGLHPAARPVSLQCRLSMCPARSLLLVATLVLL
DHLSLARNLPVATPDPGMFPCLHHSQNLLRAVSNMLQKARQTLEFYPCTS
EEIDHEDITKDKTSTVEACLPLELTKNESCLNSRETSFITNGSCLASRKT
SFMMALCLSSIYEDLKMYQVEFKTMNAKLLMDPKRQIFLDQNMLAVIDEL
MQALNFNSETVPQKSSLEEPDFYKTKIKLCILLHAFRIRAVTIDRVMSYL
NAS

The human nucleic acid sequence for IL-12 is provided below (SEQ ID NO: 9)

ATGTGGCCCCCTGGGTCAGCCTCCCAGCCACCGCCCTCACCTGCCGCGGC
CACAGGTCTGCATCCAGCGGCTCGCCCTGTGTCCCTGCAGTGCCGGCTCA
GCATGTGTCCAGCGCGCAGCCTCCTCCTTGTGGCTACCCTGGTCCTCCTG
GACCACCTCAGTTTGGCCAGAAACCTCCCCGTGGCCACTCCAGACCCAGG
AATGTTCCCATGCCTTCACCACTCCCAAAACCTGCTGAGGGCCGTCAGCA
ACATGCTCCAGAAGGCCAGACAAACTCTAGAATTTTACCCTTGCACTTCT
GAAGAGATTGATCATGAAGATATCACAAAAGATAAAACCAGCACAGTGGA
GGCCTGTTTACCATTGGAATTAACCAAGAATGAGAGTTGCCTAAATTCCA
GAGAGACCTCTTTCATAACTAATGGGAGTTGCCTGGCCTCCAGAAAGACC
TCTTTTATGATGGCCCTGTGCCTTAGTAGTATTTATGAAGACTTGAAGAT
GTACCAGGTGGAGTTCAAGACCATGAATGCAAAGCTTCTGATGGATCCTA
AGAGGCAGATCTTTCTAGATCAAAACATGCTGGCAGTTATTGATGAGCTG
ATGCAGGCCCTGAATTTCAACAGTGAGACTGTGCCACAAAAATCCTCCCT
TGAAGAACCGGATTTTTATAAAACTAAAATCAAGCTCTGCATACTTCTTC
ATGCTTTCAGAATTCGGGCAGTGACTATTGATAGAGTGATGAGCTATCTG
AATGCTTCCTAA

The human CXCL9 amino acid sequence is provided below (SEQ ID NO: 10):

MKKSGVLFLLGIILLVLIGVQGTPVVRKGRCSCISTNQGTIHLQSLKDLK
QFAPSPSCEKIEIIATLKNGVQTCLNPDSADVKELIKKWEKQVSQKKKQK
NGKKHQKKKVLKVRKSQRSRQKKTT

The human CXCL9 nucleic acid sequence is provided below (SEQ ID NO: 11); GenBank Accession No: NM_002416:

ATGAAGAAAAGTGGTGTTCTTTTCCTCTTGGGCATCATCTTGCTGGTTCT
GATTGGAGTGCAAGGAACCCCAGTAGTGAGAAAGGGTCGCTGTTCCTGCA
TCAGCACCAACCAAGGGACTATCCACCTACAATCCTTGAAAGACCTTAAA
CAATTTGCCCCAAGCCCTTCCTGCGAGAAAATTGAAATCATTGCTACACT
GAAGAATGGAGTTCAAACATGTCTAAACCCAGATTCAGCAGATGTGAAGG
AACTGATTAAAAAGTGGGAGAAACAGGTCAGCCAAAAGAAAAAGCAAAAG
AATGGGAAAAAACATCAAAAAAAGAAAGTTCTGAAAGTTCGAAAATCTCA
ACGTTCTCGTCAAAAGAAGACTACATAA

The human CD40 amino acid sequence is provided below (SEQ ID NO: 12)

MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFVLLKL

The human CD40 nucleic acid sequence is provided below (SEQ ID NO: 13); GenBank Accession No: N298241.

tttaacacag catgatcgaa acatacaacc aaacttctcc
ccgatctgcg gccactggactgcccatcag catgaaaatt
tttatgtatt tacttactgt ttacttatc
acccagatgattgggtcagc actattgct gtgtatcttc
atagaaggtt ggacaagata gaagatgaaaggaatcttca
tgaagatttt gtattcatga aaacgataca gagatgcaac
acaggagaaagatccttatc cttactgaac tgtgaggaga
ttaaaagcca gtttgaaggc tttgtgaaggatataatgtt
aaacaaagag gagacgaaga aagaaaacag ctttgaaatg
caaaaaggtgatcagaatcc tcaaattgcg gcacatgtca
taagtgaggc cagcagtaaaacaacatctgtgttacagtg
ggctgaaaaa ggatactaca ccatgagcaa caacttggta
accctggaaaatgggaaaca gctgaccgtt aaaagacaag
gactctatta tatctatgcc caagtcaccactgaccaa
tcgggaagct tcgagtcaag ctccatttat agccagcctc
tgcctaaagtcccccggtag attcgagagaatcttactcagagctg
caaatacccacagaccgccaaaccagcgggca acaatccatt
cacttgggag gagtatttga attgcaacca
ggtgcttcggtgtttgtcaa tgtgactgat ccaagccaag
tgagccatgg cactggcttc acgtcctttgtcttactcaa
actctgaaca gtgtcacctt gcaggctgtg gtggagctga
cgctgggagtc

In other examples, the protein sequence of CD40 is provided below (SEQ ID NO: 20)

MVRLPLQCVLWGCLLTAVHPEPPTACREKQYLINSQCCSLCQPGQKL
VSDCTEFTETECLPCGESEFLDTWNRETHCHQHKYCDPNLGLRVQQK
GTSETDTICTCEEGWHCTSEACESCVLHRSCSPGFGVKQIATGVSDT
ICEPCPVGFFSNVSSAFEKCHPWTSCETKDLVVQQAGTNKTDVVCGP
QDRLRALVVIPIIFGILFAILLVLVFIKKVAKKPTNKAPHPKQEPQE
INFPDDLPGSNTAAPVQETLHGCQPVTQEDGKESRISVQERQ

In other examples, the nucleic acid sequence of human CD40 is provided below (SEQ ID NO: 21); GenBank Accession No: NM_001250

ATGGTTCGTCTGCCTCTGCAGTGCGTCCTCTGGGGCTGCTTGCTGAC
CGCTGTCCATCCAGAACCACCCACTGCATGCAGAGAAAAACAGTACC
TAATAAACAGTCAGTGCTGTTCTTTGTGCCAGCCAGGACAGAAACTG
GTGAGTGACTGCACAGAGTTCACTGAAACGGAATGCCTTCCTTGCGG
TGAAAGCGAATTCCTAGACACCTGGAACAGAGAGACACACTGCCACC
AGCACAAATACTGCGACCCCAACCTAGGGCTTCGGGTCCAGCAGAAG
GGCACCTCAGAAACAGACACCATCTGCACCTGTGAAGAAGGCTGGCA
CTGTACGAGTGAGGCCTGTGAGAGCTGTGTCCTGCACCGCTCATGCT
CGCCCGGCTTTGGGGTCAAGCAGATTGCTACAGGGGTTTCTGATACC
ATCTGCGAGCCCTGCCCAGTCGGCTTCTTCTCCAATGTGTCATCTGC
TTTCGAAAAATGTCACCCTTGGACAAGCTGTGAGACCAAAGACCTGG
TTGTGCAACAGGCAGGCACAAACAAGACTGATGTTGTCTGTGGTCCC
CAGGATCGGCTGAGAGCCCTGGTGGTGATCCCCATCATCTTCGGGAT
CCTGTTTGCCATCCTCTTGGTGCTGGTCTTTATCAAAAAGGTGGCCA
AGAAGCCAACCAATAAGGCCCCCCACCCCAAGCAGGAACCCCAGGAG
ATCAATTTTCCCGACGATCTTCCTGGCTCCAACACTGCTGCTCCAGT
GCAGGAGACTTTACATGGATGCCAACCGGTCACCCAGGAGGATGGCA
AAGAGAGTCGCATCTCAGTGCAGGAGAGACAGTGA

In embodiments, as described herein, proteins can be downregulated in DCs to enhance DC functionality. For example, YTH N6-Methyladenosine RNA Binding Protein 1 (YTHDF1) promotes antigen degradation. Soluporation of molecules that downregulate expression of YTHDF1, such as siRNA or gene editing systems such as CRISPR Cas9, may enhance DC functionality. Another example is knockdown of Programmed death-ligand 1 (PD-L1) and Programmed death-ligand 2 (PD-L2) which could improve T cell activation by DCs.

The human YTHDF1 amino acid sequence is provided below (SEQ ID NO: 14)

MSATSVDTQRTKGQDNKVQNGSLHQKDTVHDNDPEPYLTGQSNQSNS
YPSMSDPYLSSYYPPSIGFPYSLNEAPWSTAGDPPIPYLTTYGQLSN
GDHHFMHDAVFGQPGGLGNNIYQHRFNFPPENPAFSAWGTSGSQGQQ
TQSSAYGSSYTYPPSSLGGTVVDGQPGFHSDTLSKAPGMNSLEQGMV
GLKIGDVSSSAVKTVGSVVSSVALTGVLSGNGGTNVNMPVSKPTSWA
AIASKPAKPQPKMKTKSGPVMGGGLPPPPIKHNMDIGTWDNKGPVPK
APVPQQAPSPQAAPQPQQVAQPLPAQPPALAQPQYQSPQQPPQTRWV
APRNRNAAFGQSGGAGSDSNSPGNVQPNSAPSVESHPVLEKLKAAHS
YNPKEFEWNLKSGRVFIIKSYSEDDIHRSIKYSIWCSTEHGNKRLDS
AFRCMSSKGPVYLLFSVNGSGHFCGVAEMKSPVDYGTSAGVWSQDKW
KGKFDVQWIFVKDVPNNQLRHIRLENNDNKPVTNSRDTQEVPLEKAK
QVLKIISSYKHTTSIFDDFAHYEKRQEEEEVVRKERQSRNKQ

The human YTHDF1 nucleic acid sequence is provided below (SEQ ID NO: 15); GenBank Accession No: NM_017798

ATGTCGGCCACCAGCGTGGACACCCAGAGAACAAAAGGACAAGATAA
TAAAGTACAAAATGGTTCGTTACATCAGAAGGATACAGTTCATGACA
ATGACTTTGAGCCCTACCTTACTGGACAGTCAAATCAGAGTAACAGT
TACCCCTCAATGAGCGACCCCTACCTGTCCAGCTATTACCCGCCGTC
CATTGGATTTCCTTACTCCCTCAATGAGGCTCCGTGGTCTACTGCAG
GGGACCCTCCGATTCCATACCTCACCACCTACGGACAGCTCAGTAAC
GGAGACCATCATTTTATGCACGATGCTGTTTTTGGGCAGCCTGGGGG
CCTGGGGAACAACATCTATCAGCACAGGTTCAATTTTTTCCCTGAAA
ACCCTGCGTTCTCAGCATGGGGGACAAGTGGGTCTCAAGGTCAGCAG
ACCCAGAGCTCCGCGTATGGGAGCAGCTACACCTACCCCCCGAGCTC
CCTGGGTGGCACGGTGGTTGATGGGCAGCCAGGCTTTCACAGCGACA
CCCTCAGCAAGGCCCCCGGGATGAACAGCCTGGAGCAGGGCATGGTT
GGCCTGAAGATTGGGGACGTCAGCTCCTCCGCCGTCAAGACGGTGGG
CTCTGTCGTCAGCAGCGTGGCACTGACTGGTGTCCTTTCTGGCAACG
GTGGGACAAATGTGAACATGCCAGTTTCAAAGCCGACCTCGTGGGCT
GCCATTGCCAGCAAGCCTGCAAAACCACAGCCTAAAATGAAAACAAA
GAGCGGGCCTGTCATGGGGGGTGGGCTGCCCCCTCCACCCATAAAGC
ATAACATGGACATTGGCACCTGGGATAACAAGGGGCCTGTGCCGAAG
GCCCCAGTCCCCCAGCAGGCACCCTCTCCACAGGCTGCCCCACAGCC
CCAGCAGGTGGCTCAGCCTCTCCCAGCACAGCCCCCAGCTTTGGCTC
AACCGCAGTATCAGAGCCCTCAGCAGCCACCCCAGACCCGCTGGGTT
GCCCCACGCAACAGAAACGCGGCGTTTGGGCAGAGCGGAGGGGCTGG
CAGCGATAGCAACTCTCCTGGAAACGTCCAGCCTAATTCTGCCCCCA
GCGTCGAATCCCACCCCGTCCTTGAAAAACTGAAGGCTGCTCACAGC
TACAACCCGAAAGAGTTTGAGTGGAATCTGAAAAGCGGGCGTGTGTT
CATCATCAAGAGCTACTCTGAGGACGACATCCACCGCTCCATTAAGT
ACTCCATCTGGTGTAGCACAGAGCACGGCAACAAGCGCCTGGACAGC
GCCTTCCGCTGCATGAGCAGCAAGGGGCCCGTCTACCTGCTCTTCAG
CGTCAATGGGAGTGGGCATTTTTGTGGGGTGGCCGAGATGAAGTCCC
CCGTGGACTACGGCACCAGTGCCGGGGTCTGGTCTCAGGACAAGTGG
AAGGGGAAGTTTGATGTCCAGTGGATTTTTGTTAAGGATGTACCCAA
TAACCAGCTCCGGCACATCAGGCTGGAGAATAACGACAACAAACCGG
TCACAAACTCCCGGGACACCCAGGAGGTGCCCTTAGAAAAAGCCAAG
CAAGTGCTGAAAATTATCAGTTCCTACAAGCACACAACCTCCATCTT
CGACGACTTTGCTCACTACGAGAAGCGCCAGGAGGAGGAGGAGGTGG
TGCGCAAGGAACGGCAGAGTCGAAACAAACAATGA

The human PD-L1 amino acid sequence is provided below (SEQ ID NO: 16)

MRIFAVFIFMTYWHLLNAFTVTVPKDLYVVEYGSNMTIECKFPVEKQ
LDLAALIVYWEMEDKNIIQFVHGEEDLKVQHSSYRQRARLLKDQLSL
GNAALQITDVKLQDAGVYRCMISYGGADYKRITVKVNAPYNKINQRI
LVVDPVTSEHELTCQAEGYPKAEVIWTSSDHQVLSGKTTTTNSKREE
KLFNVTSTLRINTTTNEIFYCTFRRLDPEENHTAELVIPELPLAHPP
NERTHLVILGAILLCLGVALTFIFRLRKGRMMDVKKCGIQDTNSKKQ
SDTHLEET

The human PD-L1 nucleic acid sequence is provided below (SEQ ID NO: 17); GenBank Accession No: NM 014143.4

ATGAGGATATTTGCTGTCTTTATATTCATGACCTACTGGCATTTGCT
GAACGCATTTACTGTCACGGTTCCCAAGGACCTATATGTGGTAGAGT
ATGGTAGCAATATGACAATTGAATGCAAATTCCCAGTAGAAAAACAA
TTAGACCTGGCTGCACTAATTGTCTATTGGGAAATGGAGGATAAGAA
CATTATTCAATTTGTGCATGGAGAGGAAGACCTGAAGGTTCAGCATA
GTAGCTACAGACAGAGGGCCCGGCTGTTGAAGGACCAGCTCTCCCTG
GGAAATGCTGCACTTCAGATCACAGATGTGAAATTGCAGGATGCAGG
GGTGTACCGCTGCATGATCAGCTATGGTGGTGCCGACTACAAGCGAA
TTACTGTGAAAGTCAATGCCCCATACAACAAAATCAACCAAAGAATT
TTGGTTGTGGATCCAGTCACCTCTGAACATGAACTGACATGTCAGGC
TGAGGGCTACCCCAAGGCCGAAGTCATCTGGACAAGCAGTGACCATC
AAGTCCTGAGTGGTAAGACCACCACCACCAATTCCAAGAGAGAGGAG
AAGCTTTTCAATGTGACCAGCACACTGAGAATCAACACAACAACTAA
TGAGATTTTCTACTGCACTTTTAGGAGATTAGATCCTGAGGAAAACC
ATACAGCTGAATTGGTCATCCCAGAACTACCTCTGGCACATCCTCCA
AATGAAAGGACTCACTTGGTAATTCTGGGAGCCATCTTATTATGCCT
TGGTGTAGCACTGACATTCATCTTCCGTTTAAGAAAAGGGAGAATGA
TGGATGTGAAAAAATGTGGCATCCAAGATACAAACTCAAAGAAGCAA
AGTGATACACATTTGGAGGAGACGTAA

The human PD-L2 amino acid sequence is provided below (SEQ ID NO: 18)

MIFLLLMLSLELQLHQIAALFTVTVPKELYIIEHGSNVTLECNFDTG
SHVNLGAITASLQKVENDTSPHRERATLLEEQLPLGKASFHIPQVQV
RDEGQYQCIIIYGVAWDYKYLTLKVKASYRKINTHILKVPETDEVEL
TCQATGYPLAEVSWPNVSVPANTSHSRTPEGLYQVTSVLRLKPPPGR
NFSCVFWNTHVRELTLASIDLQSQMEPRTHPTWLLHIFIPFCIIAFI
FIATVIALRKQLCQKLYSSKDTTKRPVTTTKREVNSAI

The human PD-L2 nucleic acid sequence is provided below (SEQ ID NO: 19); GenBank Accession No: NM_025239

ATGATCTTCCTCCTGCTAATGTTGAGCCTGGAATTGCAGCTTCACCA
GATAGCAGCTTTATTCACAGTGACAGTCCCTAAGGAACTGTACATAA
TAGAGCATGGCAGCAATGTGACCCTGGAATGCAACTTTGACACTGGA
AGTCATGTGAACCTTGGAGCAATAACAGCCAGTTTGCAAAAGGTGGA
AAATGATACATCCCCACACCGTGAAAGAGCCACTTTGCTGGAGGAGC
AGCTGCCCCTAGGGAAGGCCTCGTTCCACATACCTCAAGTCCAAGTG
AGGGACGAAGGACAGTACCAATGCATAATCATCTATGGGGTCGCCTG
GGACTACAAGTACCTGACTCTGAAAGTCAAAGCTTCCTACAGGAAAA
TAAACACTCACATCCTAAAGGTTCCAGAAACAGATGAGGTAGAGCTC
ACCTGCCAGGCTACAGGTTATCCTCTGGCAGAAGTATCCTGGCCAAA
CGTCAGCGTTCCTGCCAACACCAGCCACTCCAGGACCCCTGAAGGCC
TCTACCAGGTCACCAGTGTTCTGCGCCTAAAGCCACCCCCTGGCAGA
AACTTCAGCTGTGTGTTCTGGAATACTCACGTGAGGGAACTTACTTT
GGCCAGCATTGACCTTCAAAGTCAGATGGAACCCAGGACCCATCCAA
CTTGGCTGCTTCACATTTTCATCCCCTTCTGCATCATTGCTTTCATT
TTCATAGCCACAGTGATAGCCCTAAGAAAACAACTCTGTCAAAAGCT
GTATTCTTCAAAAGACACAACAAAAAGACCTGTCACCACAACAAAGA
GGGAAGTGAACAGTGCTATCTGA

The amino acid sequence of human CD70 is provided below (SEQ ID NO: 22)

MPEEGSGCSVRRRPYGCVLRAALVPLVAGLVICLVVCIQRFAQAQQQ
LPLESLGWDVAELQLNHTGPQQDPRLYWQGGPALGRSFLHGPELDKG
QLRIHRDGIYMVHIQVTLAICSSTTASRHHPTTLAVGICSPASRSIS
LLRLSFHQGCTIASQRLTPLARGDTLCTNLTGTLLPSRNTDETFFGV
QWVRP

The nucleic acid sequence of human CD70 is provided below (SEQ ID NO: 23); Gen Bank Accession No: NM_001252

ATGCCGGAGGAGGGTTCGGGCTGCTCGGTGCGGCGCAGGCCCTATGG
GTGCGTCCTGCGGGCTGCTTTGGTCCCATTGGTCGCGGGCTTGGTGA
TCTGCCTCGTGGTGTGCATCCAGCGCTTCGCACAGGCTCAGCAGCAG
CTGCCGCTCGAGTCACTTGGGTGGGACGTAGCTGAGCTGCAGCTGAA
TCACACAGGACCTCAGCAGGACCCCAGGCTATACTGGCAGGGGGGCC
CAGCACTGGGCCGCTCCTTCCTGCATGGACCAGAGCTGGACAAGGGG
CAGCTACGTATCCATCGTGATGGCATCTACATGGTACACATCCAGGT
GACGCTGGCCATCTGCTCCTCCACGACGGCCTCCAGGCACCACCCCA
CCACCCTGGCCGTGGGAATCTGCTCTCCCGCCTCCCGTAGCATCAGC
CTGCTGCGTCTCAGCTTCCACCAAGGTTGTACCATTGCCTCCCAGCG
CCTGACGCCCCTGGCCCGAGGGGACACACTCTGCACCAACCTCACTG
GGACACTTTTGCCTTCCCGAAACACTGATGAGACCTTCTTTGGAGTG
CAGTGGGTGCGCCCCTGA

In embodiments, the functionally closed SOLUPORE™ system is deployed to effect needle-needle near-patient cell engineering of a vaccine-size dose of engineered cells.

In other embodiments, the SOLUPORE™ system is used as described herein to generate DC vaccines for other infectious diseases as well as non-infectious diseases such as cancer.

In embodiments, other delivery methods and/or vectors are used to generate DCs as outlined herein such as viral transduction, electroporation, lipofection, nanoparticles, magnetofection, cell squeezing, carrier molecules (e.g. Feldan shuttle technology), Poros technology, Ntrans technology, microinjection, microfluidic vortex shedding.

In embodiments, the method for engineering dendritic cells to present a payload includes an mRNA encoding for severe acute respiratory syndrome coronavirus 2 (SARS-CoV-2) spike protein (SEQ ID NO: 1), or a fragment thereof as the payload. For example, the payload includes mRNA encoding for a SARS-CoV-2 spike (S) protein variant.

In examples, the payload includes full length spike protein (SEQ ID NO: 1), or subunit 1 of spike protein (SEQ ID NO: 3), or subunit 2 of spike protein (SEQ ID NO: 4).

In embodiments, the variant includes mutations of SEQ ID NO: 1 (spike protein) including K417N, E484K, N501Y, K417T, E484K, and/or N501Y of SEQ ID NO: 1. In other examples, the variant includes K417N, K417T, N439K, L452R, Y453F, S477N, E484K, N501Y, D253G, L18F, R246I, L452R, P681H, A701V, Q677P, and/or Q677H of SEQ ID NO: 1.

In further examples, the payload of the engineered dendritic cells includes mRNA encoding for at least one of cluster of differentiation 40 ligand (CD40), constitutively active Toll receptor 4 (caTLR4), and/or cluster of differentiation 70 (CD70).

Additionally, the payload of the engineered DCs of the invention may further include Snap Receptor Protein (SNARE) protein, wherein the SNARE protein includes vesicle-trafficking protein SEC22B (SEC22B). For example, the payload may include DNA or mRNA encoding SNARE or SEC22b.

In further embodiments, the methods herein provide for engineered DCs that have enhanced functionality and T cell response compared to control DCs (control DCs do not comprise a payload). Accordingly, a method of loading of mRNA into (dendritic cells) DCs ex vivo, followed by re-infusion of the transfected cells; and second, direct parenteral injection of mRNA with or without a carrier, and thus engineering the DCs such that the DCs (i) present coronavirus antigens and (ii) have enhanced functionality. The method provides for delivering the cargo or payload (e.g., coronavirus antigens, conventional mRNA molecules, synthetic mRNAs, DNA-encoding antigens or SARS-CoV-2 proteins or peptides) across a plasma membrane of a dendritic cell, comprising the steps of providing a population of dendritic cells and contacting the population of cells with a volume of an isotonic aqueous solution, the aqueous solution including the payload and an alcohol at greater than 2 percent (v/v) concentration e.g., the concentration of alcohol is greater than 5 percent (v/v) concentration. For example, the alcohol comprises ethanol, e.g., greater than 10% ethanol. In some examples, the aqueous solution comprises between 20-30% ethanol, e.g., 27% ethanol. In other examples, the alcohol comprises alcohol at a concentration less than 5 percent (v/v) concentration, e.g., zero percent alcohol. In embodiments, the alcohol is at a concentration from about 2-20% (v/v). For example, the alcohol comprises ethanol at a concentration of about 12% (v/v).

The aqueous solution for delivering cargo to cells comprises a physiologically-acceptable salt, e.g., potassium chloride (KCl) in between 12.5-500 mM, e.g., 25-250 mM, 50-275 mM, 50-200 mM, 50-150 mM, 50-125 mM For example, the solution is isotonic with respect to the cytoplasm of a mammalian cell such a human dendritic cell. Such an exemplary isotonic delivery solution comprises about 106 mM KCl, e.g., 106 nM KCl.

The methods are used to deliver any cargo molecule or molecules to mammalian cells, e.g., mammalian immune cells such as antigen presenting cells, e.g., dendritic cells (DCs).

In other embodiments, additional mammalian cells are used, including for example, adherent or non-adherent and are particularly useful to deliver cargo to non-adherent cells because of the difficulties associated with doing so prior to the invention. In some examples, the non-adherent cell comprises a peripheral blood mononuclear cell, e.g., the non-adherent cell comprises an immune cell such as a T cell (T lymphocyte). An immune cell such as a T cell is optionally activated with a ligand of cluster of differentiation 3 (CD3), cluster of differentiation 28 (CD28), or a combination thereof. For example, the ligand is an antibody or antibody fragment that binds to CD3 or CD28 or both.

The method involves delivering the cargo in the delivery solution to a population of dendritic cells comprising a monolayer. For example, the monolayer is contacted with a spray of aqueous delivery solution. The method delivers the payload/cargo (compound or composition) into the cytoplasm of the cell and wherein the population of cells comprises a greater percent viability compared to delivery of the payload by electroporation or nucleofection—a significant advantage of the SOLUPORE™ system.

Any compound or composition can be delivered. For example, the payload comprises coronavirus antigens, conventional mRNA molecules, synthetic mRNAs, DNA-encoding antigens or SARS-CoV-2 proteins or peptides. Additionally, the payload may include a messenger ribonucleic acid (mRNA), e.g., a mRNA that encodes a gene-editing composition. For example, the gene editing composition reduces the expression of an immune checkpoint inhibitor such as PD-1 or PD-L1. In some examples, the mRNA encodes a chimeric antigen receptor (CAR).

In certain embodiments, the monolayer of dendritic cells resides on a membrane filter. In some embodiments, the membrane filter is vibrated following contacting the cell monolayer with a spray of the delivery solution. The membrane filter may be vibrated or agitated before, during, and/or after spraying the cells with the delivery solution.

Also within the invention is a system comprising: a housing configured to receive a plate comprising a well; a differential pressure applicator configured to apply a differential pressure to the well; a delivery solution applicator configured to deliver atomized delivery solution to the well; a stop solution applicator configured to deliver a stop solution to the well; and a culture medium applicator configured to deliver a culture medium to the well. A stop solution is one that lacks a cell membrane permeabilizing agent, e.g., ethanol. An example phosphate buffered saline or any physiologically-compatible buffer solution. The system optionally further comprises: an addressable well assembly configured to: align the differential pressure applicator adjacent the well for applying the differential pressure to the well; align the delivery solution applicator adjacent the well for delivering the atomized delivery solution to the well; align the stop solution applicator adjacent the well to deliver the stop solution to the well; and/or align the culture medium applicator adjacent the well to deliver the culture medium to the well.

The addressable well assembly can include a movable base-plate configured to receive the plate comprising the well and move the plate in at least one dimension. The addressable well assembly can include a mounting assembly configured to couple to the delivery solution applicator, the stop solution applicator and the culture medium applicator.

The delivery solution applicator can include a nebulizer. The delivery solution applicator can be configured to deliver 10-300 micro liters of the delivery solution per actuation.

The system can include a temperature control system configured to control a temperature of the delivery solution and/or of the plate comprising the well.

The system can include an enclosure configured to control an environment of the plate comprising the well.

The differential pressure applicator can include a nozzle assembly configured to form a seal with an opening of the well and to deliver a vapor to the well to increase or decrease pressure within the well, thereby driving a liquid portion of the culture medium from the well such that a layer of cells remains within the well.

The stop solution applicator can comprise a needle emitter configured to couple to a stop solution reservoir.

The culture medium applicator can comprise a needle emitter configured to couple to a culture medium reservoir.

The system can further comprise a controller configured to: receive user input; operate the delivery solution applicator to deliver the atomized delivery solution to a cellular monolayer within the well; incubate, for a first incubation period, the cellular monolayer after application of the delivery solution; operate, in response to expiration of the first incubation period, the stop solution applicator to deliver the stop solution to the cellular monolayer; and incubate, for a second incubation period and in response to application of the stop solution, the cellular monolayer. The controller can be further configured to: iterate operation of the delivery solution applicator, incubation for the first incubation period, operation of the stop solution applicator, and incubation for the second incubation period for a predetermined number of iterations.

The system can further comprise a controller configured to: operate the positive pressure system to remove supernatant from the well to create a cellular monolayer within the well.

The delivery solution applicator can include a spray head and a collar encircling a distal end of the spray head, wherein the collar is configured to prevent contamination between wells in a multi-well plate, wherein the collar is configured to provide a gap between the plate and the collar.

The delivery solution applicator can include a spray head and a film encircling a distal end of the spray head.

The system can further comprise a vibration system coupled to a membrane holder and configured to vibrate a membrane.

The system can further comprise the plate, wherein the well is configured to contain a population of dendritic cells.

The delivery solution includes an isotonic aqueous solution, the aqueous solution including the payload and an alcohol at greater than 5 percent (v/v) concentration. The alcohol can comprise ethanol. The aqueous solution can comprise greater than 10% ethanol. The aqueous solution can comprise between 20-30% ethanol, e.g., 20-27% v/v ethanol. The aqueous solution can comprise 27% ethanol. The aqueous solution can comprise between 12.5-500 mM KCl. The aqueous solution can comprise between 106 mM KCl. In other embodiments, the alcohol comprises less than 5% concentration (v/v), including for example, zero percent alcohol.

The payload can comprise coronavirus antigens, conventional mRNA molecules, synthetic mRNAs, DNA-encoding antigens or SARS-CoV-2 proteins or peptides. Additional examples include messenger ribonucleic acid (mRNA). The mRNA can encode a gene-editing composition. For example, the gene editing composition reduces the expression of PD-1. The mRNA can encode a chimeric antigen receptor.

The system is used to deliver a cargo compound or composition to a mammalian cell (e.g., a dendritic cell).

In another aspect, a composition comprises an isotonic aqueous solution, the aqueous solution comprising KCl at a concentration of 10-500 mM and ethanol at greater than 5 percent (v/v) concentration for use to deliver a cargo compound or composition to a mammalian cell. The KCl concentration can be 106 mM and the alcohol concentration can be 27%. In embodiments, the alcohol (e.g., ethanol) can be less than 5 percent (v/v) concentration. For example, the KCl concentration can be about 106 mM and the alcohol concentration can be about 12% v/v.

The compounds that are loaded into the composition are processed or purified. For example, polynucleotides, polypeptides, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its natural-occurring state. A purified or isolated polypeptide is free of the amino acids or sequences that flank it in its naturally-occurring state. Purified also defines a degree of sterility that is safe for administration to a human subject, e.g., lacking infectious or toxic agents. In the case of tumor antigens, the antigen may be purified or a processed preparation such as a tumor cell lysate.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “about” in reference to a given parameter or other measurable factor means within 10%.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. For example, the base sequence is the spike protein SEQ ID NO: 1, SEQ ID NO: 30, SEQ ID NO: 3 and SEQ. ID NO: 4.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof, e.g., the base sequence is the spike protein SEQ ID NO: 1, SEQ ID NO: 30, SEQ ID NO: 3 and SEQ. ID NO: 4.), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1, −2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is an image depicting an autologous cell based vaccine delivery method described herein.

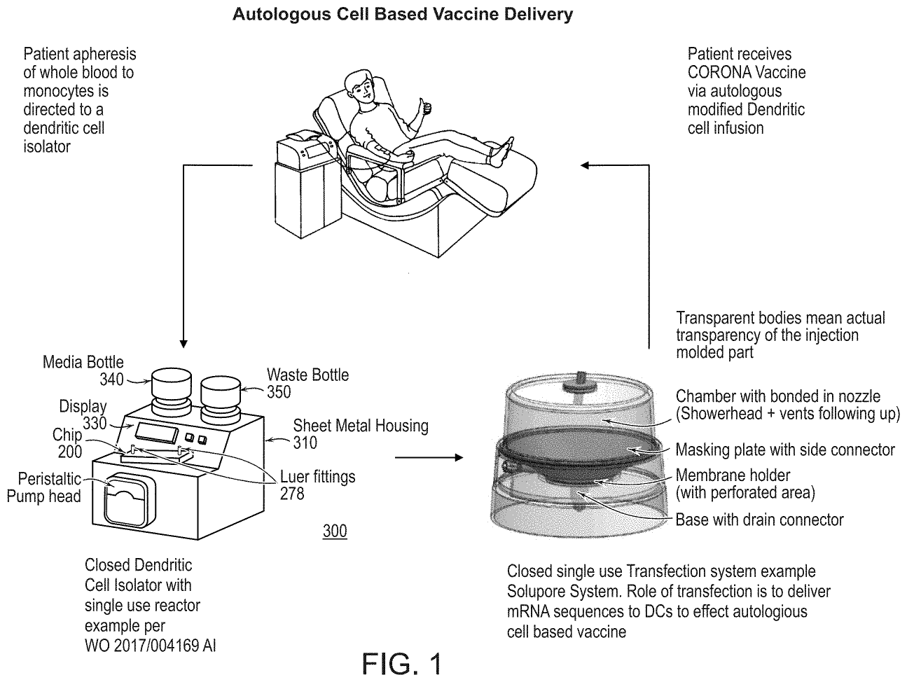

FIG. 2 is an image depicting an allogenaeic cell based vaccine delivery method described herein.

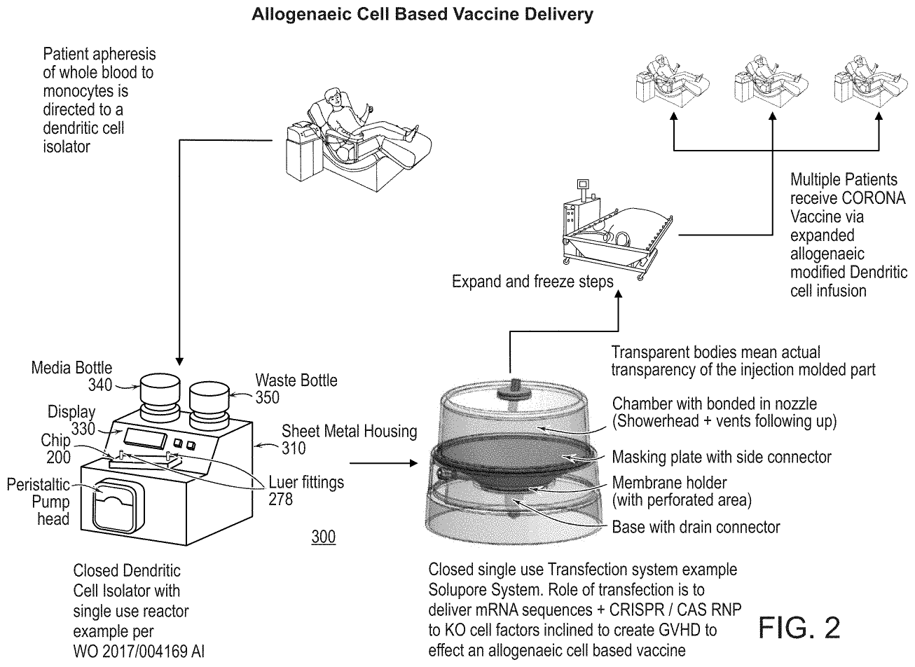

FIG. 3 is an image depicting alternative methods of cell based vaccine delivery methods described herein.

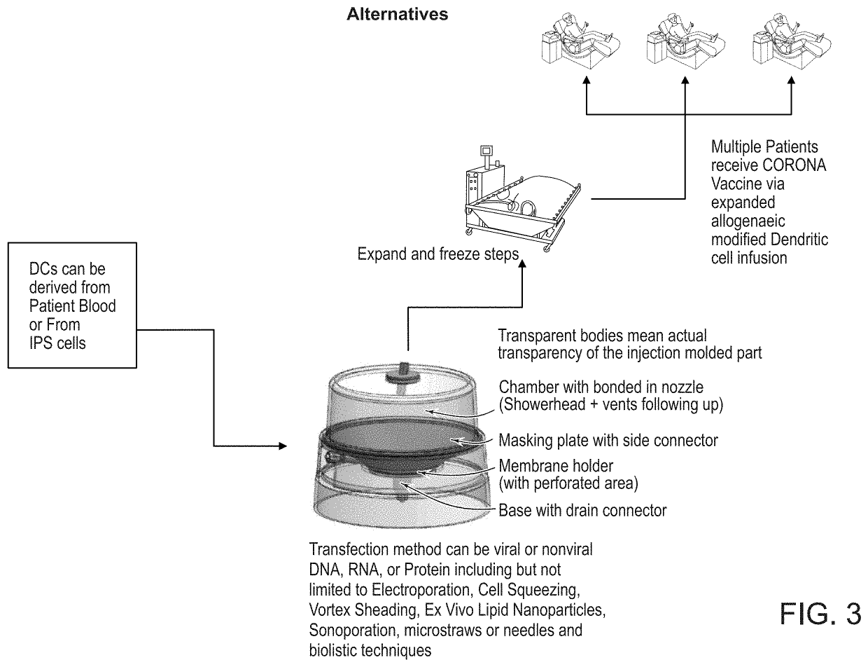

FIG. 4 is an image depicting autologous cell based vaccine methods manufactured at Contract Development Manufacturing Organization (CDMO), as described herein.

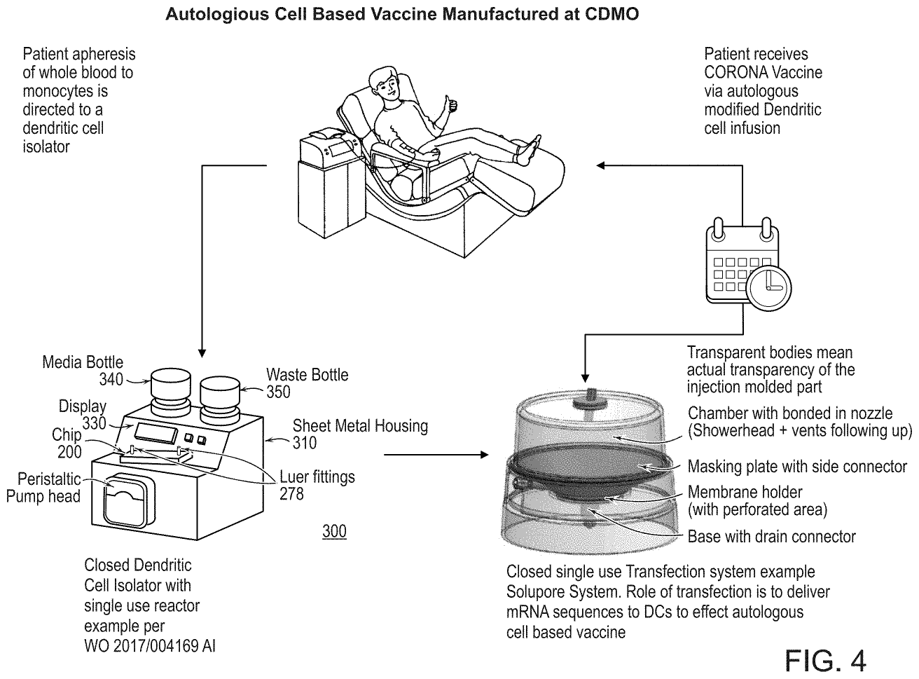

FIG. 5 is a schematic depicting the major targets used in COVID vaccine candidates. Severe acute respiratory syndrome coronavirus 2 (SARS-CoV-2) contains four major structure proteins: spike (S), membrane (M) and envelope (E) proteins, which are embedded on the virion surface, and nucleocapsid (N) protein, which binds viral RNA inside the virion. The S protein trimer in its pre-fusion conformation is shown. The S protein comprises the 51 subunit (which includes the N-terminal domain (NTD) and the receptor-binding domain (RBD)) (the receptor-binding motif (RBM) within the RBD is also labelled) and the S2 subunit (which includes fusion peptide (FP), connecting region (CR), heptad repeat 1 (HR1), heptad repeat (HR2) and central helix (CH)). The SARS-CoV-2 S protein binds to its host receptor, the dimeric human angiotensin-converting enzyme 2 (hACE2), via the RBD and dissociates the 51 subunits. Cleavage at both S1-S2 and ST sites allows structural rearrangement of the S2 subunit required for virus-host membrane fusion. The S2-trimer in its post-fusion arrangement is shown. The RBD is an attractive vaccine target. The generation of an RBD-dimer or RBD-trimer has been shown to enhance the immunogenicity of RBD-based vaccines. A stabilized S-trimer shown with a C-terminal trimer-tag is a vaccine target. The pre-fusion S protein is generally metastable during in vitro preparations and prone to transform into its post-fusion conformation. Mutation of two residues (K986 and V987) to proline stabilizes S protein (S-2P) and prevents the pre-fusion to post-fusion structural change. The schematic was taken from: Dai L, Gao G F. Viral targets for vaccines against COVID-19. Nat Rev Immunol. 2021 February; 21(2):73-82. doi: 10.1038/s41577-020-00480-0. Epub 2020 Dec. 18. PMID: 33340022; PMCID: PMC7747004.

### DETAILED DESCRIPTION

Severe acute respiratory syndrome (SARS) is a viral respiratory illness caused by a coronavirus called SARS-associated coronavirus (SARS-CoV). SARS-CoV-2 is a new coronavirus that is responsible for the 2020 COVID-19 global pandemic. A vaccine is not currently available for COVID-19 and is urgently required. A vaccine is a biological preparation that provides active acquired immunity to a particular infectious disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future.

The invention relates to methods of engineering cells (e.g., dendritic cells (DCs)) for vaccines (e.g., to generate COVID-19-specific immunity). The DC processing method utilizes transient cell membrane permeabilization. The invention is based on the surprising discovery that the SOLUPORE™ system can be used to engineer DCs such that the DCs (i) present coronavirus antigens and (ii) have enhanced functionality, e.g., ability to present antigen encoded by the delivered nucleic acid and the development of an improved immune response to the antigen. These vaccines are generated using the SOLUPORE™ system to deliver mRNA encoding for SARS-CoV-2 antigens to autologous or allogeneic dendritic cells ex vivo.

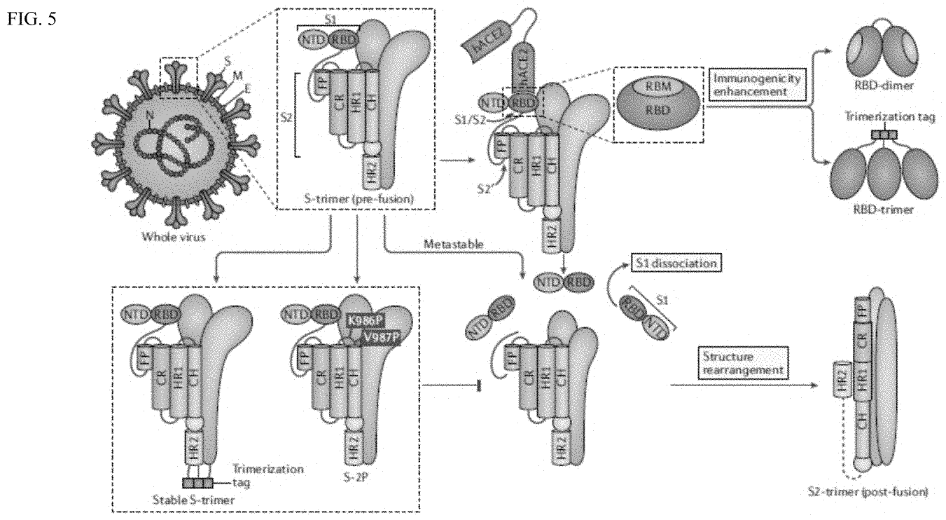

SARS-CoV-2 is an enveloped single stranded RNA (ssRNA) virus with spike-like-glycoproteins expressed on the surface forming a ‘corona’. The whole genome sequence (29,903 nt) has been assigned GenBank accession number MN908947 (SEQ ID NO: 2). SARS-CoV-2 consists of four key proteins (FIG. 5). The S (“spike”) protein (NCBI GenBank Ref. No: QHD43416.1) enables the attachment and entry of SARS-CoV-2 to the host cells [S protein sequence provided below (SEQ ID NO: 1)].

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpl nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvngnaqaln |
| 961 | tlvkqlssnf gaissvlndi lsrldkveae vqidrlitgr lqslqtyvtq qliraaeira |
| 1021 | sanlaatkms ecvlgqskry dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeqyi kwpwyiwlgf iagliaivmv timlccmtsc csclkgccsc gscckfdedd |
| 1261 | sepvlkgvkl hyt |

Exemplary landmark residues, domains, and fragments of Spike (S) protein include, but are not limited to residues 13-304 (N-terminal domain of the 51 subunit), subunit 1 (51 SEQ ID NO: 3), and subunit 2 (S2; SEQ ID NO: 4).

S1 (Subunit 1 of Spike protein)
(SEQ ID NO: 3)
mfvflvllpl vssqcvnltt rtqlppaytn sftrgvyypd
kvfrssvlhs tqdlflpffs nvtwfhaihv sgtngtkrfd
npvlpfndgv yfasteksni irgwifgttl dsktqslliv
nnatnvvikv cefqfcndpf lgvyyhknnk swmesefrvy
ssannctfey vsqpflmdle gkqgnfknlr efvfknidgy
fkiyskhtpi nlvrdlpqgf saleplvdlp iginitrfqt
llalhrsylt pgdsssgwta gaaayyvgyl qprtfllkyn
engtitdavd caldplsetk ctlksftvek giyqtsnfrv
qptesivrfp nitnlcpfge vfnatrfasv yawnrkrisn
cvadysvlyn sasfstfkcy gvsptklndl cftnvyadsf
virgdevrqi apgqtgkiad ynyklpddft gcviawnsnn
ldskvggnyn ylyrlfrksn lkpferdist eiyqagstpc
ngvegfncyf plqsygfqpt ngvgyqpyrv vvlsfellha
patvcgpkks tnlvknkcvn fn
S2 (Subunit 2 of Spike protein and S1/S2
cleavage region)
SEQ ID NO: 4
fngltgtg vltesnkkfl pfqqfgrdia dttdavrdpq
tleilditpc sfggvsvitp gtntsnqvav lyqdvnctev
pvaihadqlt ptwrvystgs nvfqtragcl igaehvnnsy
ecdipigagi casyqtqtns prrarsvasq siiaytmslg
aensvaysnn siaiptnfti svtteilpvs mtktsvdctm
yicgdstecs nlllqygsfc tqlnraltgi aveqdkntqe
vfaqvkqiyk tppikdfggf nfsqilpdps kpskrsfied
llfnkvtlad agfikqygdc lgdiaardli caqkfngltv
lpplltdemi aqytsallag titsgwtfga gaalqipfam
qmayrfngig vtqnvlyenq klianqfnsa igkiqdslss
tasalgklqd vvnqnaqaln tlvkqlssnf gaissvindi
lsrldkveae vqidrlitgr lqslqtyvtq qliraaeira
sanlaatkms ecvlgqskrv dfcgkgyhlm sfpqsaphgv
vflhvtyvpa qeknfttapa ichdgkahfp regvfvsngt
hwfvtqrnfy epqiittdnt fvsgncdvvi givnntvydp
lqpeldsfke eldkyfknht spdvdlgdis ginasvvniq
keidrlneva knlneslidl qelgkyeqyi kwpwyiwlgf
iagliaivmv timlccmtsc csclkgccsc gscckfdedd
sepvlkgvkl hyt

A fragment of an S protein is less than the length of the full length protein, e.g., a fragment is at least 3, 4, 5, 6, 7, 8, 9, 10, 20, 30, 40, 50, 100, 200 or more residues in length, but less than e.g., 1273 residues in the case of full length S1 above. Compared with the sequence shown above (SEQ ID NO: 1-S protein sequence), these variants have the following mutations: N501Y in B.1.1.7 (the UK “Kent” variant); K417N, E484K, and N501Y in B.1.351 (South Africa variant); and K417T, E484K, and N501Y in P.1 (Brazil variant); see Zhou D., Evidence of escape of SARS-CoV-2 variant B.1.351 from natural and vaccine-indice sera. Cell. 2021. 189:1-14. These mutations are shown in bold and underlined above (in SEQ ID NO:1).

A spike protein variant is also contemplated in the invention (e.g., as the payload for delivery to the dendritic cells). An exemplary spike protein variant amino acid sequence is provided below, which is a D614G variant meaning the amino acid ‘D’ at position 614 is changed to amino acid ‘G’).

| (SEQ ID NO: 5) | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyry vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqgvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvlndi lsrldkveae vqidrlitgr lqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeqyi kwpwyiwlgf iagliaivmv timlccmtsc csclkgccsc gscckfdedd |
| 1261 | sepvlkgvkl hyt |

Additional spike protein variants include K417N, K417T, N439K, L452R, Y453F, S477N, E484K, N501Y, D253G, L18F, R246I, L452R, P681H, A701V, Q677P, or Q677H of SEQ ID NO: 1.

The nucleic acid sequence of the full virus (NCBI GenBank Ref No: MN908947.3 SEQ ID NO: 2) is provided below, and the start and stop codons bold and underlined.

| | |
|---|---|
| 1 | attaaaggtt tataccttcc caggtaacaa accaaccaac tttcgatctc ttgtagatct |
| 61 | gttctctaaa cgaactttaa aatctgtgtg gctgtcactc ggctgcatgc ttagtgcact |
| 121 | cacgcagtat aattaataac taattactgt cgttgacagg acacgagtaa ctcgtctatc |
| 181 | ttctgcaggc tgcttacggt ttcgtccgtg ttgcagccga tcatcagcac atctaggttt |
| 241 | cgtccgggtg tgaccgaaag gtaagatgga gagccttgtc cctggtttca acgagaaaac |
| 301 | acacgtccaa ctcagtttgc ctgttttaca ggttcgcgac gtgctcgtac gtggctttgg |
| 361 | agactccgtg gaggaggtct tatcagaggc acgtcaacat cttaaagatg gcacttgtgg |
| 421 | cttagtagaa gttgaaaaag gcgttttgcc tcaacttgaa cagccctatg tgttcatcaa |
| 481 | acgttcggat gctcgaactg cacctcatgg tcatgttatg gttgagctgg tagcagaact |
| 541 | cgaaggcatt cagtacggtc gtagtggtga gacacttggt gtccttgtcc ctcatgtggg |
| 601 | cgaaatacca gtggcttacc gcaaggttct tcttcgtaag aacggtaata aaggagctgg |
| 661 | tggccatagt tacggcgccg atctaaagtc atan ttgactta ggcgacgagcttggcactga |
| 721 | tccttatgaa gattttcaag aaaactggaa cactaaacat agcagtggtg ttacccgtga |
| 781 | actcatgcgt gagcttaacg gaggggcata cactcgctat gtcgataaca acttctgtgg |
| 841 | ccctgatggc taccctcttg agtgcattaa agaccttcta gcacgtgctg gtaaagcttc |
| 901 | atgcactttg tccgaacaac tggactttat tgacactaag aggggtgtat actgctgccg |
| 961 | tgaacatgag catgaaattg cttggtacac ggaacgttct gaaaagagct atgaattgca |
| 1021 | gacacctttt gaaattaaat tggcaaagaa atttgacacc ttcaatgggg aatgtccaaa |
| 1081 | ttttgtattt cccttaaatt ccataatcaa gactattcaa ccaagggttg aaaagaaaaa |
| 1141 | gcttgatggc tttatgggta gaattcgatc tgtctatcca gttgcgtcac caaatgaatg |
| 1201 | caaccaaatg tgcctttcaa ctctcatgaa gtgtgatcat tgtggtgaaa cttcatggca |
| 1261 | gacgggcgat tttgttaaag ccacttgcga attttgtggc actgagaatt tgactaaaga |
| 1321 | aggtgccact acttgtggtt acttacccca aaatgctgtt gttaaaattt attgtccagc |
| 1381 | atgtcacaat tcagaagtag gacctgagca tagtcttgcc gaataccata atgaatctgg |
| 1441 | cttgaaaacc attcttcgta agggtggtcg cactattgcc tttggaggct gtgtgttctc |
| 1501 | ttatgttggt tgccataaca agtgtgccta ttgggttcca cgtgctagcg ctaacatagg |
| 1561 | ttgtaaccat acaggtgttg ttggagaagg ttccgaaggt cttaatgaca accttcttga |
| 1621 | aatactccaa aaagagaaag tcaacatcaa tattgttggt gactttaaac ttaatgaaga |
| 1681 | gatcgccatt attttggcat ctttttctgc ttccacaagt gcttttgtgg aaactgtgaa |
| 1741 | aggtttggat tataaagcat tcaaacaaat tgttgaatcc tgtggtaatt ttaaagttac |
| 1801 | aaaaggaaaa gctaaaaaag gtgcctggaa tattggtgaa cagaaatcaa tactgagtcc |
| 1861 | tctttatgca tttgcatcag aggctgctcg tgttgtacga tcaattttct cccgcactct |
| 1921 | tgaaactgct caaaattctg tgcgtgtttt acagaaggcc gctataacaa tactagatgg |
| 1981 | aatttcacag tattcactga gactcattga tgctatgatg ttcacatctg atttggctac |
| 2041 | taacaatcta gttgtaatgg cctacattac aggtggtgtt gttcagttga cttcgcagtg |
| 2101 | gctaactaac atctttggca ctgtttatga aaaactcaaa cccgtccttg attggcttga |
| 2161 | agagaagttt aaggaaggtg tagagtttct tagagacggt tgggaaattg ttaaatttat |
| 2221 | ctcaacctgt gcttgtgaaa ttgtcggtgg acaaattgtc acctgtgcaa aggaaattaa |
| 2281 | ggagagtgtt cagacattct ttaagcttgt aaataaattt ttggctttgt gtgctgactc |
| 2341 | tatcattatt ggtggagcta aacttaaagc cttgaattta ggtgaaacat ttgtcacgca |
| 2401 | ctcaaaggga ttgtacagaa agtgtgttaa atccagagaa gaaactggcc tactcatgcc |
| 2461 | tctaaaagcc ccaaaagaaa ttatcttctt agagggagaa acacttccca cagaagtgtt |
| 2521 | aacagaggaa gttgtcttga aaactggtga tttacaacca ttagaacaac ctactagtga |
| 2581 | agctgttgaa gctccattgg ttggtacacc agtttgtatt aacgggctta tgttgctcga |
| 2641 | aatcaaagac acagaaaagt actgtgccct tgcacctaat atgatggtaa caaacaatac |
| 2701 | cttcacactc aaaggcggtg caccaacaaa ggttactttt ggtgatgaca ctgtgataga |
| 2761 | agtgcaaggt tacaagagtg tgaatatcac ttttgaactt gatgaaagga ttgataaagt |
| 2821 | acttaatgag aagtgctctg cctatacagt tgaactcggt acagaagtaa atgagttcgc |
| 2881 | ctgtgttgtg gcagatgctg tcataaaaac tttgcaacca gtatctgaat tacttacacc |
| 2941 | actgggcatt gatttagatg agtggagtat ggctacatac tacttatttg atgagtctgg |
| 3001 | tgagtttaaa ttggcttcac atatgtattg ttctttctac cctccagatg aggatgaaga |
| 3061 | agaaggtgat tgtgaagaag aagagtttga gccatcaact caatatgagt atggtactga |
| 3121 | agatgattac caaggtaaac ctttggaatt tggtgccact tctgctgctc ttcaacctga |
| 3181 | agaagagcaa gaagaagatt ggttagatga tgatagtcaa caaactgttg gtcaacaaga |
| 3241 | cggcagtgag gacaatcaga caactactat tcaaacaatt gttgaggttc aacctcaatt |
| 3301 | agagatggaa cttacaccag ttgttcagac tattgaagtg aatagtttta gtggttattt |
| 3361 | aaaacttact gacaatgtat acattaaaaa tgcagacatt gtggaagaag ctaaaaaggt |
| 3421 | aaaaccaaca gtggttgtta atgcagccaa tgtttacctt aaacatggag gaggtgttgc |
| 3481 | aggagcctta aataaggcta ctaacaatgc catgcaagtt gaatctgatg attacatagc |
| 3541 | tactaatgga ccacttaaag tgggtggtag ttgtgtttta agcggacaca atcttgctaa |
| 3601 | acactgtctt catgttgtcg gcccaaatgt taacaaaggt gaagacattc aacttcttaa |
| 3661 | gagtgcttat gaaaatttta atcagcacga agttctactt gcaccattat tatcagctgg |
| 3721 | tatttttggt gctgacccta tacattcttt aagagtttgt gtagatactg ttcgcacaaa |
| 3781 | tgtctactta gctgtctttg ataaaaatct ctatgacaaa cttgtttcaa gctttttgga |
| 3841 | aatgaagagt gaaaagcaag ttgaacaaaa gatcgctgag attcctaaag aggaagttaa |
| 3901 | gccatttata actgaaagta aaccttcagt tgaacagaga aaacaagatg ataagaaaat |
| 3961 | caaagcttgt gttgaagaag ttacaacaac tctggaagaa actaagttcc tcacagaaaa |
| 4021 | cttgttactt tatattgaca ttaatggcaa tcttcatcca gattctgcca ctcttgttag |
| 4081 | tgacattgac atcactttct taaagaaaga tgctccatat atagtgggtg atgttgttca |
| 4141 | agagggtgtt ttaactgctg tggttatacc tactaaaaag gctggtggca ctactgaaat |
| 4201 | gctagcgaaa gctttgagaa aagtgccaac agacaattat ataaccactt acccgggtca |
| 4261 | gggtttaaat ggttacactg tagaggaggc aaagacagtg cttaaaaagt gtaaaagtgc |
| 4321 | cttttacatt ctaccatcta ttatctctaa tgagaagcaa gaaattcttg gaactgtttc |
| 4381 | ttggaatttg cgagaaatgc ttgcacatgc agaagaaaca cgcaaattaa tgcctgtctg |
| 4441 | tgtggaaact aaagccatag tttcaactat acagcgtaaa tataagggta ttaaaataca |
| 4501 | agagggtgtg gttgattatg gtgctagatt ttacttttac accagtaaaa caactgtagc |
| 4561 | gtcacttatc aacacactta acgatctaaa tgaaactctt gttacaatgc cacttggcta |
| 4621 | tgtaacacat ggcttaaatt tggaagaagc tgctcggtat atgagatctc tcaaagtgcc |
| 4681 | agctacagtt tctgtttctt cacctgatgc tgttacagcg tataatggtt atcttacttc |
| 4741 | ttcttctaaa acacctgaag aacattttat tgaaaccatc tcacttgctg gttcctataa |
| 4801 | agattggtcc tattctggac aatctacaca actaggtata gaatttctta agagaggtga |
| 4861 | taaaagtgta tattacacta gtaatcctac cacattccac ctagatggtg aagttatcac |
| 4921 | ctttgacaat cttaagacac ttctttcttt gagagaagtg aggactatta aggtgtttac |
| 4981 | aacagtagac aacattaacc tccacacgca agttgtggac atgtcaatga catatggaca |
| 5041 | acagtttggt ccaacttatt tggatggagc tgatgttact aaaataaaac ctcataattc |
| 5101 | acatgaaggt aaaacatttt atgttttacc taatgatgac actctacgtg ttgaggcttt |
| 5161 | tgagtactac cacacaactg atcctagttt tctgggtagg tacatgtcag cattaaatca |
| 5221 | cactaaaaag tggaaatacc cacaagttaa tggtttaact tctattaaat gggcagataa |
| 5281 | caactgttat cttgccactg cattgttaac actccaacaa atagagttga agtttaatcc |
| 5341 | acctgctcta caagatgctt attacagagc aagggctggt gaagctgcta acttttgtgc |
| 5401 | acttatctta gcctactgta ataagacagt aggtgagtta ggtgatgtta gagaaacaat |
| 5461 | gagttacttg tttcaacatg ccaatttaga ttcttgcaaa agagtcttga acgtggtgtg |
| 5521 | taaaacttgt ggacaacagc agacaaccct taagggtgta gaagctgtta tgtacatggg |
| 5581 | cacactttct tatgaacaat ttaagaaagg tgttcagata ccttgtacgt gtggtaaaca |
| 5641 | agctacaaaa tatctagtac aacaggagtc accttttgtt atgatgtcag caccacctgc |
| 5701 | tcagtatgaa cttaagcatg gtacatttac ttgtgctagt gagtacactg gtaattacca |
| 5761 | gtgtggtcac tataaacata taacttctaa agaaactttg tattgcatag acggtgcttt |
| 5821 | acttacaaag tcctcagaat acaaaggtcc tattacggat gttttctaca aagaaaacag |
| 5881 | ttacacaaca accataaaac cagttactta taaattggat ggtgttgttt gtacagaaat |
| 5941 | tgaccctaag ttggacaatt attataagaa agacaattct tatttcacag agcaaccaat |
| 6001 | tgatcttgta ccaaaccaac catatccaaa cgcaagcttc gataatttta agtttgtatg |
| 6061 | tgataatatc aaatttgctg atgatttaaa ccagttaact ggttataaga aacctgcttc |
| 6121 | aagagagctt aaagttacat ttttccctga cttaaatggt gatgtggtgg ctattgatta |
| 6181 | taaacactac acaccctctt ttaagaaagg agctaaattg ttacataaac ctattgtttg |
| 6241 | gcatgttaac aatgcaacta ataaagccac gtataaacca aatacctggt gtatacgttg |
| 6301 | tctttggagc acaaaaccag ttgaaacatc aaattcgttt gatgtactga agtcagagga |
| 6361 | cgcgcaggga atggataatc ttgcctgcga agatctaaaa ccagtctctg aagaagtagt |
| 6421 | ggaaaatcct accatacaga aagacgttct tgagtgtaat gtgaaaacta ccgaagttgt |
| 6481 | aggagacatt atacttaaac cagcaaataa tagtttaaaa attacagaag aggttggcca |
| 6541 | cacagatcta atggctgctt atgtagacaa ttctagtctt actattaaga aacctaatga |
| 6601 | attatctaga gtattaggtt tgaaaaccct tgctactcat ggtttagctg ctgttaatag |
| 6661 | tgtcccttgg gatactatag ctaattatgc taagcctttt cttaacaaag ttgttagtac |
| 6721 | aactactaac atagttacac ggtgtttaaa ccgtgtttgt actaattata tgccttattt |
| 6781 | ctttacttta ttgctacaat tgtgtacttt tactagaagt acaaattcta gaattaaagc |
| 6841 | atctatgccg actactatag caaagaatac tgttaagagt gtcggtaaat tttgtctaga |
| 6901 | ggcttcattt aattatttga agtcacctaa tttttctaaa ctgataaata ttataatttg |
| 6961 | gtttttacta ttaagtgttt gcctaggttc tttaatctac tcaaccgctg ctttaggtgt |
| 7021 | tttaatgtct aatttaggca tgccttctta ctgtactggt tacagagaag gctatttgaa |
| 7081 | ctctactaat gtcactattg caacctactg tactggttct ataccttgta gtgtttgtct |
| 7141 | tagtggttta gattctttag acacctatcc ttctttagaa actatacaaa ttaccatttc |
| 7201 | atcttttaaa tgggatttaa ctgcttttgg cttagttgca gagtggtttt tggcatatat |
| 7261 | tcttttcact aggtttttct atgtacttgg attggctgca atcatgcaat tgtttttcag |
| 7321 | ctattttgca gtacatttta ttagtaattc ttggcttatg tggttaataa ttaatcttgt |
| 7381 | acaaatggcc ccgatttcag ctatggttag aatgtacatc ttctttgcat cattttatta |
| 7441 | tgtatggaaa agttatgtgc atgttgtaga cggttgtaat tcatcaactt gtatgatgtg |
| 7501 | ttacaaacgt aatagagcaa caagagtcga atgtacaact attgttaatg gtgttagaag |
| 7561 | gtccttttat gtctatgcta atggaggtaa aggcttttgc aaactacaca attggaattg |
| 7621 | tgttaattgt gatacattct gtgctggtag tacatttatt agtgatgaag ttgcgagaga |
| 7681 | cttgtcacta cagtttaaaa gaccaataaa tcctactgac cagtcttctt acatcgttga |
| 7741 | tagtgttaca gtgaagaatg gttccatcca tctttacttt gataaagctg gtcaaaagac |
| 7801 | ttatgaaaga cattctctct ctcattttgt taacttagac aacctgagag ctaataacac |
| 7861 | taaaggttca ttgcctatta atgttatagt ttttgatggt aaatcaaaat gtgaagaatc |
| 7921 | atctgcaaaa tcagcgtctg tttactacag tcagcttatg tgtcaaccta tactgttact |
| 7981 | agatcaggca ttagtgtctg atgttggtga tagtgcggaa gttgcagtta aaatgtttga |
| 8041 | tgcttacgtt aatacgtttt catcaacttt taacgtacca atggaaaaac tcaaaacact |
| 8101 | agttgcaact gcagaagctg aacttgcaaa gaatgtgtcc ttagacaatg tcttatctac |
| 8161 | ttttatttca gcagctcggc aagggtttgt tgattcagat gtagaaacta aagatgttgt |
| 8221 | tgaatgtctt aaattgtcac atcaatctga catagaagtt actggcgata gttgtaataa |
| 8281 | ctatatgctc acctataaca aagttgaaaa catgacaccc cgtgaccttg gtgcttgtat |
| 8341 | tgactgtagt gcgcgtcata ttaatgcgca ggtagcaaaa agtcacaaca ttgctttgat |
| 8401 | atggaacgtt aaagatttca tgtcattgtc tgaacaacta cgaaaacaaa tacgtagtgc |
| 8461 | tgctaaaaag aataacttac cttttaagtt gacatgtgca actactagac aagttgttaa |
| 8521 | tgttgtaaca acaaagatag cacttaaggg tggtaaaatt gttaataatt ggttgaagca |
| 8581 | gttaattaaa gttacacttg tgttcctttt tgttgctgct attttctatt taataacacc |
| 8641 | tgttcatgtc atgtctaaac atactgactt ttcaagtgaa atcataggat acaaggctat |
| 8701 | tgatggtggt gtcactcgtg acatagcatc tacagatact tgttttgcta acaaacatgc |
| 8761 | tgattttgac acatggttta gccagcgtgg tggtagttat actaatgaca aagcttgccc |
| 8821 | attgattgct gcagtcataa caagagaagt gggttttgtc gtgcctggtt tgcctggcac |
| 8881 | gatattacgc acaactaatg gtgacttttt gcatttctta cctagagttt ttagtgcagt |
| 8941 | tggtaacatc tgttacacac catcaaaact tatagagtac actgactttg caacatcagc |
| 9001 | ttgtgttttg gctgctgaat gtacaatttt taaagatgct tctggtaagc cagtaccata |
| 9061 | ttgttatgat accaatgtac tagaaggttc tgttgcttat gaaagtttac gccctgacac |
| 9121 | acgttatgtg ctcatggatg gctctattat tcaatttcct aacacctacc ttgaaggttc |
| 9181 | tgttagagtg gtaacaactt ttgattctga gtactgtagg cacggcactt gtgaaagatc |
| 9241 | agaagctggt gtttgtgtat ctactagtgg tagatgggta cttaacaatg attattacag |
| 9301 | atctttacca ggagttttct gtggtgtaga tgctgtaaat ttacttacta atatgtttac |
| 9361 | accactaatt caacctattg gtgctttgga catatcagca tctatagtag ctggtggtat |
| 9421 | tgtagctatc gtagtaacat gccttgccta ctattttatg aggtttagaa gagcttttgg |
| 9481 | tgaatacagt catgtagttg cctttaatac tttactattc cttatgtcat tcactgtact |
| 9541 | ctgtttaaca ccagtttact cattcttacc tggtgtttat tctgttattt acttgtactt |
| 9601 | gacattttat cttactaatg atgtttcttt tttagcacat attcagtgga tggttatgtt |
| 9661 | cacaccttta gtacctttct ggataacaat tgcttatatc atttgtattt ccacaaagca |
| 9721 | tttctattgg ttctttagta attacctaaa gagacgtgta gtctttaatg gtgtttcctt |
| 9781 | tagtactttt gaagaagctg cgctgtgcac ctttttgtta aataaagaaa tgtatctaaa |
| 9841 | gttgcgtagt gatgtgctat tacctcttac gcaatataat agatacttag ctctttataa |
| 9901 | taagtacaag tattttagtg gagcaatgga tacaactagc tacagagaag ctgcttgttg |
| 9961 | tcatctcgca aaggctctca atgacttcag taactcaggt tctgatgttc tttaccaacc |
| 10021 | accacaaacc tctatcacct cagctgtttt gcagagtggt tttagaaaaa tggcattccc |
| 10081 | atctggtaaa gttgagggtt gtatggtaca agtaacttgt ggtacaacta cacttaacgg |
| 10141 | tctttggctt gatgacgtag tttactgtcc aagacatgtg atctgcacct ctgaagacat |
| 10201 | gcttaaccct aattatgaag atttactcat tcgtaagtct aatcataatt tcttggtaca |
| 10261 | ggctggtaat gttcaactca gggttattgg acattctatg caaaattgtg tacttaagct |
| 10321 | taaggttgat acagccaatc ctaagacacc taagtataag tttgttcgca ttcaaccagg |
| 10381 | acagactttt tcagtgttag cttgttacaa tggttcacca tctggtgttt accaatgtgc |
| 10441 | tatgaggccc aatttcacta ttaagggttc attccttaat ggttcatgtg gtagtgttgg |
| 10501 | ttttaacata gattatgact gtgtctcttt ttgttacatg caccatatgg aattaccaac |
| 10561 | tggagttcat gctggcacag acttagaagg taacttttat ggaccttttg ttgacaggca |
| 10621 | aacagcacaa gcagctggta cggacacaac tattacagtt aatgttttag cttggttgta |
| 10681 | cgctgctgtt ataaatggag acaggtggtt tctcaatcga tttaccacaa ctcttaatga |
| 10741 | ctttaacctt gtggctatga agtacaatta tgaacctcta acacaagacc atgttgacat |
| 10801 | actaggacct ctttctgctc aaactggaat tgccgtttta gatatgtgtg cttcattaaa |
| 10861 | agaattactg caaaatggta tgaatggacg taccatattg ggtagtgctt tattagaaga |
| 10921 | tgaatttaca ccttttgatg ttgttagaca atgctcaggt gttactttcc aaagtgcagt |
| 10981 | gaaaagaaca atcaagggta cacaccactg gttgttactc acaattttga cttcactttt |
| 11041 | agttttagtc cagagtactc aatggtcttt gttctttttt ttgtatgaaa atgccttttt |
| 11101 | accttttgct atgggtatta ttgctatgtc tgcttttgca atgatgtttg tcaaacataa |
| 11161 | gcatgcattt ctctgtttgt ttttgttacc ttctcttgcc actgtagctt attttaatat |
| 11221 | ggtctatatg cctgctagtt gggtgatgcg tattatgaca tggttggata tggttgatac |
| 11281 | tagtttgtct ggttttaagc taaaagactg tgttatgtat gcatcagctg tagtgttact |
| 11341 | aatccttatg acagcaagaa ctgtgtatga tgatggtgct aggagagtgt ggacacttat |
| 11401 | gaatgtcttg acactcgttt ataaagttta ttatggtaat gctttagatc aagccatttc |
| 11461 | catgtgggct cttataatct ctgttacttc taactactca ggtgtagtta caactgtcat |
| 11521 | gtttttggcc agaggtattg tttttatgtg tgttgagtat tgccctattt tcttcataac |
| 11581 | tggtaataca cttcagtgta taatgctagt ttattgtttc ttaggctatt tttgtacttg |
| 11641 | ttactttggc ctcttttgtt tactcaaccg ctactttaga ctgactcttg gtgtttatga |
| 11701 | ttacttagtt tctacacagg agtttagata tatgaattca cagggactac tcccacccaa |
| 11761 | gaatagcata gatgccttca aactcaacat taaattgttg ggtgttggtg gcaaaccttg |
| 11821 | tatcaaagta gccactgtac agtctaaaat gtcagatgta aagtgcacat cagtagtctt |
| 11881 | actctcagtt ttgcaacaac tcagagtaga atcatcatct aaattgtggg ctcaatgtgt |
| 11941 | ccagttacac aatgacattc tcttagctaa agatactact gaagcctttg aaaaaatggt |
| 12001 | ttcactactt tctgttttgc tttccatgca gggtgctgta gacataaaca agctttgtga |
| 12061 | agaaatgctg gacaacaggg caaccttaca agctatagcc tcagagttta gttcccttcc |
| 12121 | atcatatgca gcttttgcta ctgctcaaga agcttatgag caggctgttg ctaatggtga |
| 12181 | ttctgaagtt gttcttaaaa agttgaagaa gtctttgaat gtggctaaat ctgaatttga |
| 12241 | ccgtgatgca gccatgcaac gtaagttgga aaagatggct gatcaagcta tgacccaaat |
| 12301 | gtataaacag gctagatctg aggacaagag ggcaaaagtt actagtgcta tgcagacaat |
| 12361 | gcttttcact atgcttagaa agttggataa tgatgcactc aacaacatta tcaacaatgc |
| 12421 | aagagatggt tgtgttccct tgaacataat acctcttaca acagcagcca aactaatggt |
| 12481 | tgtcatacca gactataaca catataaaaa tacgtgtgat ggtacaacat ttacttatgc |
| 12541 | atcagcattg tgggaaatcc aacaggttgt agatgcagat agtaaaattg ttcaacttag |
| 12601 | tgaaattagt atggacaatt cacctaattt agcatggcct cttattgtaa cagctttaag |
| 12661 | ggccaattct gctgtcaaat tacagaataa tgagcttagt cctgttgcac tacgacagat |
| 12721 | gtcttgtgct gccggtacta cacaaactgc ttgcactgat gacaatgcgt tagcttacta |
| 12781 | caacacaaca aagggaggta ggtttgtact tgcactgtta tccgatttac aggatttgaa |
| 12841 | atgggctaga ttccctaaga gtgatggaac tggtactatc tatacagaac tggaaccacc |
| 12901 | ttgtaggttt gttacagaca cacctaaagg tcctaaagtg aagtatttat actttattaa |
| 12961 | aggattaaac aacctaaata gaggtatggt acttggtagt ttagctgcca cagtacgtct |
| 13021 | acaagctggt aatgcaacag aagtgcctgc caattcaact gtattatctt tctgtgcttt |
| 13081 | tgctgtagat gctgctaaag cttacaaaga ttatctagct agtgggggac aaccaatcac |
| 13141 | taattgtgtt aagatgttgt gtacacacac tggtactggt caggcaataa cagttacacc |
| 13201 | ggaagccaat atggatcaag aatcctttgg tggtgcatcg tgttgtctgt actgccgttg |
| 13261 | ccacatagat catccaaatc ctaaaggatt ttgtgactta aaaggtaagt atgtacaaat |
| 13321 | acctacaact tgtgctaatg accctgtggg ttttacactt aaaaacacag tctgtaccgt |
| 13381 | ctgcggtatg tggaaaggtt atggctgtag ttgtgatcaa ctccgcgaac ccatgcttca |
| 13441 | gtcagctgat gcacaatcgt ttttaaacgg gtttgcggtg taagtgcagc ccgtcttaca |
| 13501 | ccgtgcggca caggcactag tactgatgtc gtatacaggg cttttgacat ctacaatgat |
| 13561 | aaagtagctg gttttgctaa attcctaaaa actaattgtt gtcgcttcca agaaaaggac |
| 13621 | gaagatgaca atttaattga ttcttacttt gtagttaaga gacacacttt ctctaactac |
| 13681 | caacatgaag aaacaattta taatttactt aaggattgtc cagctgttgc taaacatgac |
| 13741 | ttctttaagt ttagaataga cggtgacatg gtaccacata tatcacgtca acgtcttact |
| 13801 | aaatacacaa tggcagacct cgtctatgct ttaaggcatt ttgatgaagg taattgtgac |
| 13861 | acattaaaag aaatacttgt cacatacaat tgttgtgatg atgattattt caataaaaag |
| 13921 | gactggtatg attttgtaga aaacccagat atattacgcg tatacgccaa cttaggtgaa |
| 13981 | cgtgtacgcc aagctttgtt aaaaacagta caattctgtg atgccatgcg aaatgctggt |
| 14041 | attgttggtg tactgacatt agataatcaa gatctcaatg gtaactggta tgatttcggt |
| 14101 | gatttcatac aaaccacgcc aggtagtgga gttcctgttg tagattctta ttattcattg |
| 14161 | ttaatgccta tattaacctt gaccagggct ttaactgcag agtcacatgt tgacactgac |
| 14221 | ttaacaaagc cttacattaa gtgggatttg ttaaaatatg acttcacgga agagaggtta |
| 14281 | aaactctttg accgttattt taaatattgg gatcagacat accacccaaa ttgtgttaac |
| 14341 | tgtttggatg acagatgcat tctgcattgt gcaaacttta atgttttatt ctctacagtg |
| 14401 | ttcccaccta caagttttgg accactagtg agaaaaatat ttgttgatgg tgttccattt |
| 14461 | gtagtttcaa ctggatacca cttcagagag ctaggtgttg tacataatca ggatgtaaac |
| 14521 | ttacatagct ctagacttag ttttaaggaa ttacttgtgt atgctgctga ccctgctatg |
| 14581 | cacgctgctt ctggtaatct attactagat aaacgcacta cgtgcttttc agtagctgca |
| 14641 | cttactaaca atgttgcttt tcaaactgtc aaacccggta attttaacaa agacttctat |
| 14701 | gactttgctg tgtctaaggg tttctttaag gaaggaagtt ctgttgaatt aaaacacttc |
| 14761 | ttctttgctc aggatggtaa tgctgctatc agcgattatg actactatcg ttataatcta |
| 14821 | ccaacaatgt gtgatatcag acaactacta tttgtagttg aagttgttga taagtacttt |
| 14881 | gattgttacg atggtggctg tattaatgct aaccaagtca tcgtcaacaa cctagacaaa |
| 14941 | tcagctggtt ttccatttaa taaatggggt aaggctagac tttattatga ttcaatgagt |
| 15001 | tatgaggatc aagatgcact tttcgcatat acaaaacgta atgtcatccc tactataact |
| 15061 | caaatgaatc ttaagtatgc cattagtgca aagaatagag ctcgcaccgt agctggtgtc |
| 15121 | tctatctgta gtactatgac caatagacag tttcatcaaa aattattgaa atcaatagcc |
| 15181 | gccactagag gagctactgt agtaattgga acaagcaaat tctatggtgg ttggcacaac |
| 15241 | atgttaaaaa ctgtttatag tgatgtagaa aaccctcacc ttatgggttg ggattatcct |
| 15301 | aaatgtgata gagccatgcc taacatgctt agaattatgg cctcacttgt tcttgctcgc |
| 15361 | aaacatacaa cgtgttgtag cttgtcacac cgtttctata gattagctaa tgagtgtgct |
| 15421 | caagtattga gtgaaatggt catgtgtggc ggttcactat atgttaaacc aggtggaacc |
| 15481 | tcatcaggag atgccacaac tgcttatgct aatagtgttt ttaacatttg tcaagctgtc |
| 15541 | acggccaatg ttaatgcact tttatctact gatggtaaca aaattgccga taagtatgtc |
| 15601 | cgcaatttac aacacagact ttatgagtgt ctctatagaa atagagatgt tgacacagac |
| 15661 | tttgtgaatg agttttacgc atatttgcgt aaacatttct caatgatgat actctctgac |
| 15721 | gatgctgttg tgtgtttcaa tagcacttat gcatctcaag gtctagtggc tagcataaag |
| 15781 | aactttaagt cagttcttta ttatcaaaac aatgttttta tgtctgaagc aaaatgttgg |
| 15841 | actgagactg accttactaa aggacctcat gaattttgct ctcaacatac aatgctagtt |
| 15901 | aaacagggtg atgattatgt gtaccttcct tacccagatc catcaagaat cctaggggcc |
| 15961 | ggctgttttg tagatgatat cgtaaaaaca gatggtacac ttatgattga acggttcgtg |
| 16021 | tctttagcta tagatgctta cccacttact aaacatccta atcaggagta tgctgatgtc |
| 16081 | tttcatttgt acttacaata cataagaaag ctacatgatg agttaacagg acacatgtta |
| 16141 | gacatgtatt ctgttatgct tactaatgat aacacttcaa ggtattggga acctgagttt |
| 16201 | tatgaggcta tgtacacacc gcatacagtc ttacaggctg ttggggcttg tgttctttgc |
| 16261 | aattcacaga cttcattaag atgtggtgct tgcatacgta gaccattctt atgttgtaaa |
| 16321 | tgctgttacg accatgtcat atcaacatca cataaattag tcttgtctgt taatccgtat |
| 16381 | gtttgcaatg ctccaggttg tgatgtcaca gatgtgactc aactttactt aggaggtatg |
| 16441 | agctattatt gtaaatcaca taaaccaccc attagttttc cattgtgtgc taatggacaa |
| 16501 | gtttttggtt tatataaaaa tacatgtgtt ggtagcgata atgttactga ctttaatgca |
| 16561 | attgcaacat gtgactggac aaatgctggt gattacattt tagctaacac ctgtactgaa |
| 16621 | agactcaagc tttttgcagc agaaacgctc aaagctactg aggagacatt taaactgtct |
| 16681 | tatggtattg ctactgtacg tgaagtgctg tctgacagag aattacatct ttcatgggaa |
| 16741 | gttggtaaac ctagaccacc acttaaccga aattatgtct ttactggtta tcgtgtaact |
| 16801 | aaaaacagta aagtacaaat aggagagtac acctttgaaa aaggtgacta tggtgatgct |
| 16861 | gttgtttacc gaggtacaac aacttacaaa ttaaatgttg gtgattattt tgtgctgaca |
| 16921 | tcacatacag taatgccatt aagtgcacct acactagtgc cacaagagca ctatgttaga |
| 16981 | attactggct tatacccaac actcaatatc tcagatgagt tttctagcaa tgttgcaaat |
| 17041 | tatcaaaagg ttggtatgca aaagtattct acactccagg gaccacctgg tactggtaag |
| 17101 | agtcattttg ctattggcct agctctctac tacccttctg ctcgcatagt gtatacagct |
| 17161 | tgctctcatg ccgctgttga tgcactatgt gagaaggcat taaaatattt gcctatagat |
| 17221 | aaatgtagta gaattatacc tgcacgtgct cgtgtagagt gttttgataa attcaaagtg |
| 17281 | aattcaacat tagaacagta tgtcttttgt actgtaaatg cattgcctga gacgacagca |
| 17341 | gatatagttg tctttgatga aatttcaatg gccacaaatt atgatttgag tgttgtcaat |
| 17401 | gccagattac gtgctaagca ctatgtgtac attggcgacc ctgctcaatt acctgcacca |
| 17461 | cgcacattgc taactaaggg cacactagaa ccagaatatt tcaattcagt gtgtagactt |
| 17521 | atgaaaacta taggtccaga catgttcctc ggaacttgtc ggcgttgtcc tgctgaaatt |
| 17581 | gttgacactg tgagtgcttt ggtttatgat aataagctta aagcacataa agacaaatca |
| 17641 | gctcaatgct ttaaaatgtt ttataagggt gttatcacgc atgatgtttc atctgcaatt |
| 17701 | aacaggccac aaataggcgt ggtaagagaa ttccttacac gtaaccctgc ttggagaaaa |
| 17761 | gctgtcttta tttcacctta taattcacag aatgctgtag cctcaaagat tttgggacta |
| 17821 | ccaactcaaa ctgttgattc atcacagggc tcagaatatg actatgtcat attcactcaa |
| 17881 | accactgaaa cagctcactc ttgtaatgta aacagattta atgttgctat taccagagca |
| 17941 | aaagtaggca tactttgcat aatgtctgat agagaccttt atgacaagtt gcaatttaca |
| 18001 | agtcttgaaa ttccacgtag gaatgtggca actttacaag ctgaaaatgt aacaggactc |
| 18061 | tttaaagatt gtagtaaggt aatcactggg ttacatccta cacaggcacc tacacacctc |
| 18121 | agtgttgaca ctaaattcaa aactgaaggt ttatgtgttg acatacctgg catacctaag |
| 18181 | gacatgacct atagaagact catctctatg atgggtttta aaatgaatta tcaagttaat |
| 18241 | ggttacccta acatgtttat cacccgcgaa gaagctataa gacatgtacg tgcatggatt |
| 18301 | ggcttcgatg tcgaggggtg tcatgctact agagaagctg ttggtaccaa tttaccttta |
| 18361 | cagctaggtt tttctacagg tgttaaccta gttgctgtac ctacaggtta tgttgataca |
| 18421 | cctaataata cagatttttc cagagttagt gctaaaccac cgcctggaga tcaatttaaa |
| 18481 | cacctcatac cacttatgta caaaggactt ccttggaatg tagtgcgtat aaagattgta |
| 18541 | caaatgttaa gtgacacact taaaaatctc tctgacagag tcgtatttgt cttatgggca |
| 18601 | catggctttg agttgacatc tatgaagtat tttgtgaaaa taggacctga gcgcacctgt |
| 18661 | tgtctatgtg atagacgtgc cacatgcttt tccactgctt cagacactta tgcctgttgg |
| 18721 | catcattcta ttggatttga ttacgtctat aatccgttta tgattgatgt tcaacaatgg |
| 18781 | ggttttacag gtaacctaca aagcaaccat gatctgtatt gtcaagtcca tggtaatgca |
| 18841 | catgtagcta gttgtgatgc aatcatgact aggtgtctag ctgtccacga gtgctttgtt |
| 18901 | aagcgtgttg actggactat tgaatatcct ataattggtg atgaactgaa gattaatgcg |
| 18961 | gcttgtagaa aggttcaaca catggttgtt aaagctgcat tattagcaga caaattccca |
| 19021 | gttcttcacg acattggtaa ccctaaagct attaagtgtg tacctcaagc tgatgtagaa |
| 19081 | tggaagttct atgatgcaca gccttgtagt gacaaagctt ataaaataga agaattattc |
| 19141 | tattcttatg ccacacattc tgacaaattc acagatggtg tatgcctatt ttggaattgc |
| 19201 | aatgtcgata gatatcctgc taattccatt gtttgtagat ttgacactag agtgctatct |
| 19261 | aaccttaact tgcctggttg tgatggtggc agtttgtatg taaataaaca tgcattccac |
| 19321 | acaccagctt ttgataaaag tgcttttgtt aatttaaaac aattaccatt tttctattac |
| 19381 | tctgacagtc catgtgagtc tcatggaaaa caagtagtgt cagatataga ttatgtacca |
| 19441 | ctaaagtctg ctacgtgtat aacacgttgc aatttaggtg gtgctgtctg tagacatcat |
| 19501 | gctaatgagt acagattgta tctcgatgct tataacatga tgatctcagc tggctttagc |
| 19561 | ttgtgggttt acaaacaatt tgatacttat aacctctgga acacttttac aagacttcag |
| 19621 | agtttagaaa atgtggcttt taatgttgta aataagggac actttgatgg acaacagggt |
| 19681 | gaagtaccag tttctatcat taataacact gtttacacaa aagttgatgg tgttgatgta |
| 19741 | gaattgtttg aaaataaaac aacattacct gttaatgtag catttgagct ttgggctaag |
| 19801 | cgcaacatta aaccagtacc agaggtgaaa atactcaata atttgggtgt ggacattgct |
| 19861 | gctaatactg tgatctggga ctacaaaaga gatgctccag cacatatatc tactattggt |
| 19921 | gtttgttcta tgactgacat agccaagaaa ccaactgaaa cgatttgtgc accactcact |
| 19981 | gtcttttttg atggtagagt tgatggtcaa gtagacttat ttagaaatgc ccgtaatggt |
| 20041 | gttcttatta cagaaggtag tgttaaaggt ttacaaccat ctgtaggtcc caaacaagct |
| 20101 | agtcttaatg gagtcacatt aattggagaa gccgtaaaaa cacagttcaa ttattataag |
| 20161 | aaagttgatg gtgttgtcca acaattacct gaaacttact ttactcagag tagaaattta |
| 20221 | caagaattta aacccaggag tcaaatggaa attgatttct tagaattagc tatggatgaa |
| 20281 | ttcattgaac ggtataaatt agaaggctat gccttcgaac atatcgttta tggagatttt |
| 20341 | agtcatagtc agttaggtgg tttacatcta ctgattggac tagctaaacg ttttaaggaa |
| 20401 | tcaccttttg aattagaaga ttttattcct atggacagta cagttaaaaa ctatttcata |
| 20461 | acagatgcgc aaacaggttc atctaagtgt gtgtgttctg ttattgattt attacttgat |
| 20521 | gattttgttg aaataataaa atcccaagat ttatctgtag tttctaaggt tgtcaaagtg |
| 20581 | actattgact atacagaaat ttcatttatg ctttggtgta aagatggcca tgtagaaaca |
| 20641 | ttttacccaa aattacaatc tagtcaagcg tggcaaccgg gtgttgctat gcctaatctt |
| 20701 | tacaaaatgc aaagaatgct attagaaaag tgtgaccttc aaaattatgg tgatagtgca |
| 20761 | acattaccta aaggcataat gatgaatgtc gcaaaatata ctcaactgtg tcaatattta |
| 20821 | aacacattaa cattagctgt accctataat atgagagtta tacattttgg tgctggttct |
| 20881 | gataaaggag ttgcaccagg tacagctgtt ttaagacagt ggttgcctac gggtacgctg |
| 20941 | cttgtcgatt cagatcttaa tgactttgtc tctgatgcag attcaacttt gattggtgat |
| 21001 | tgtgcaactg tacatacagc taataaatgg gatctcatta ttagtgatat gtacgaccct |
| 21061 | aagactaaaa atgttacaaa agaaaatgac tctaaagagg gttttttcac ttacatttgt |
| 21121 | gggtttatac aacaaaagct agctcttgga ggttccgtgg ctataaagat aacagaacat |
| 21181 | tcttggaatg ctgatcttta taagctcatg ggacacttcg catggtggac agcctttgtt |
| 21241 | actaatgtga atgcgtcatc atctgaagca tttttaattg gatgtaatta tcttggcaaa |
| 21301 | ccacgcgaac aaatagatgg ttatgtcatg catgcaaatt acatattttg gaggaataca |
| 21361 | aatccaattc agttgtcttc ctattcttta tttgacatga gtaaatttcc ccttaaatta |
| 21421 | aggggtactg ctgttatgtc tttaaaagaa ggtcaaatca atgatatgat tttatctctt |
| 21481 | cttagtaaag gtagacttat aattagagaa aacaacagag ttgttatttc tagtgatgtt |
| 21541 | cttgttaaca actaaacgaa caatgtttgt ttttcttgtt ttattgccac tagtctctag |
| 21601 | tcagtgtgtt aatcttacaa ccagaactca attaccccct gcatacacta attctttcac |
| 21661 | acgtggtgtt tattaccctg acaaagtttt cagatcctca gttttacatt caactcagga |
| 21721 | cttgttctta cctttctttt ccaatgttac ttggttccat gctatacatg tctctgggac |
| 21781 | caatggtact aagaggtttg ataaccctgt cctaccattt aatgatggtg tttattttgc |
| 21841 | ttccactgag aagtctaaca taataagagg ctggattttt ggtactactt tagattcgaa |
| 21901 | gacccagtcc ctacttattg ttaataacgc tactaatgtt gttattaaag tctgtgaatt |
| 21961 | tcaattttgt aatgatccat ttttgggtgt ttattaccac aaaaacaaca aaagttggat |
| 22021 | ggaaagtgag ttcagagttt attctagtgc gaataattgc acttttgaat atgtctctca |
| 22081 | gccttttctt atggaccttg aaggaaaaca gggtaatttc aaaaatctta gggaatttgt |
| 22141 | gtttaagaat attgatggtt attttaaaat atattctaag cacacgccta ttaatttagt |
| 22201 | gcgtgatctc cctcagggtt tttcggcttt agaaccattg gtagatttgc caataggtat |
| 22261 | taacatcact aggtttcaaa ctttacttgc tttacataga agttatttga ctcctggtga |
| 22321 | ttcttcttca ggttggacag ctggtgctgc agcttattat gtgggttatc ttcaacctag |
| 22381 | gacttttcta ttaaaatata atgaaaatgg aaccattaca gatgctgtag actgtgcact |
| 22441 | tgaccctctc tcagaaacaa agtgtacgtt gaaatccttc actgtagaaa aaggaatcta |
| 22501 | tcaaacttct aactttagag tccaaccaac agaatctatt gttagatttc ctaatattac |
| 22561 | aaacttgtgc ccttttggtg aagtttttaa cgccaccaga tttgcatctg tttatgcttg |
| 22621 | gaacaggaag agaatcagca actgtgttgc tgattattct gtcctatata attccgcatc |
| 22681 | attttccact tttaagtgtt atggagtgtc tcctactaaa ttaaatgatc tctgctttac |
| 22741 | taatgtctat gcagattcat ttgtaattag aggtgatgaa gtcagacaaa tcgctccagg |
| 22801 | gcaaactgga aagattgctg attataatta taaattacca gatgatttta caggctgcgt |
| 22861 | tatagcttgg aattctaaca atcttgattc taaggttggt ggtaattata attacctgta |
| 22921 | tagattgttt aggaagtcta atctcaaacc ttttgagaga gatatttcaa ctgaaatcta |
| 22981 | tcaggccggt agcacacctt gtaatggtgt tgaaggtttt aattgttact ttcctttaca |
| 23041 | atcatatggt ttccaaccca ctaatggtgt tggttaccaa ccatacagag tagtagtact |
| 23101 | ttcttttgaa cttctacatg caccagcaac tgtttgtgga cctaaaaagt ctactaattt |
| 23161 | ggttaaaaac aaatgtgtca atttcaactt caatggttta acaggcacag gtgttcttac |
| 23221 | tgagtctaac aaaaagtttc tgcctttcca acaatttggc agagacattg ctgacactac |
| 23281 | tgatgctgtc cgtgatccac agacacttga gattcttgac attacaccat gttcttttgg |
| 23341 | tggtgtcagt gttataacac caggaacaaa tacttctaac caggttgctg ttctttatca |
| 23401 | ggatgttaac tgcacagaag tccctgttgc tattcatgca gatcaactta ctcctacttg |
| 23461 | gcgtgtttat tctacaggtt ctaatgtttt tcaaacacgt gcaggctgtt taataggggc |
| 23521 | tgaacatgtc aacaactcat atgagtgtga catacccatt ggtgcaggta tatgcgctag |
| 23581 | ttatcagact cagactaatt ctcctcggcg ggcacgtagt gtagctagtc aatccatcat |
| 23641 | tgcctacact atgtcacttg gtgcagaaaa ttcagttgct tactctaata actctattgc |
| 23701 | catacccaca aattttacta ttagtgttac cacagaaatt ctaccagtgt ctatgaccaa |
| 23761 | gacatcagta gattgtacaa tgtacatttg tggtgattca actgaatgca gcaatctttt |
| 23821 | gttgcaatat ggcagttttt gtacacaatt aaaccgtgct ttaactggaa tagctgttga |
| 23881 | acaagacaaa aacacccaag aagtttttgc acaagtcaaa caaatttaca aaacaccacc |
| 23941 | aattaaagat tttggtggtt ttaatttttc acaaatatta ccagatccat caaaaccaag |
| 24001 | caagaggtca tttattgaag atctactttt caacaaagtg acacttgcag atgctggctt |
| 24061 | catcaaacaa tatggtgatt gccttggtga tattgctgct agagacctca tttgtgcaca |
| 24121 | aaagtttaac ggccttactg ttttgccacc tttgctcaca gatgaaatga ttgctcaata |
| 24181 | cacttctgca ctgttagcgg gtacaatcac ttctggttgg acctttggtg caggtgctgc |
| 24241 | attacaaata ccatttgcta tgcaaatggc ttataggttt aatggtattg gagttacaca |
| 24301 | gaatgttctc tatgagaacc aaaaattgat tgccaaccaa tttaatagtg ctattggcaa |
| 24361 | aattcaagac tcactttctt ccacagcaag tgcacttgga aaacttcaag atgtggtcaa |
| 24421 | ccaaaatgca caagctttaa acacgcttgt taaacaactt agctccaatt ttggtgcaat |
| 24481 | ttcaagtgtt ttaaatgata tcctttcacg tcttgacaaa gttgaggctg aagtgcaaat |
| 24541 | tgataggttg atcacaggca gacttcaaag tttgcagaca tatgtgactc aacaattaat |
| 24601 | tagagctgca gaaatcagag cttctgctaa tcttgctgct actaaaatgt cagagtgtgt |
| 24661 | acttggacaa tcaaaaagag ttgatttttg tggaaagggc tatcatctta tgtccttccc |
| 24721 | tcagtcagca cctcatggtg tagtcttctt gcatgtgact tatgtccctg cacaagaaaa |
| 24781 | gaacttcaca actgctcctg ccatttgtca tgatggaaaa gcacactttc ctcgtgaagg |
| 24841 | tgtctttgtt tcaaatggca cacactggtt tgtaacacaa aggaattttt atgaaccaca |
| 24901 | aatcattact acagacaaca catttgtgtc tggtaactgt gatgttgtaa taggaattgt |
| 24961 | caacaacaca gtttatgatc ctttgcaacc tgaattagac tcattcaagg aggagttaga |
| 25021 | taaatatttt aagaatcata catcaccaga tgttgattta ggtgacatct ctggcattaa |
| 25081 | tgcttcagtt gtaaacattc aaaaagaaat tgaccgcctc aatgaggttg ccaagaattt |
| 25141 | aaatgaatct ctcatcgatc tccaagaact tggaaagtat gagcagtata taaaatggcc |
| 25201 | atggtacatt tggctaggtt ttatagctgg cttgattgcc atagtaatgg tgacaattat |
| 25261 | gctttgctgt atgaccagtt gctgtagttg tctcaagggc tgttgttctt gtggatcctg |
| 25321 | ctgcaaattt gatgaagacg actctgagcc agtgctcaaa ggagtcaaat tacattacac |
| 25381 | ataaacgaac ttatggattt gtttatgaga atcttcacaa ttggaactgt aactttgaag |
| 25441 | caaggtgaaa tcaaggatgc tactccttca gattttgttc gcgctactgc aacgataccg |
| 25501 | atacaagcct cactcccttt cggatggctt attgttggcg ttgcacttct tgctgttttt |
| 25561 | cagagcgctt ccaaaatcat aaccctcaaa aagagatggc aactagcact ctccaagggt |
| 25621 | gttcactttg tttgcaactt gctgttgttg tttgtaacag tttactcaca ccttttgctc |
| 25681 | gttgctgctg gccttgaagc cccttttctc tatctttatg ctttagtcta cttcttgcag |
| 25741 | agtataaact ttgtaagaat aataatgagg ctttggcttt gctggaaatg ccgttccaaa |
| 25801 | aacccattac tttatgatgc caactatttt ctttgctggc atactaattg ttacgactat |
| 25861 | tgtatacctt acaatagtgt aacttcttca attgtcatta cttcaggtga tggcacaaca |
| 25921 | agtcctattt ctgaacatga ctaccagatt ggtggttata ctgaaaaatg ggaatctgga |
| 25981 | gtaaaagact gtgttgtatt acacagttac ttcacttcag actattacca gctgtactca |
| 26041 | actcaattga gtacagacac tggtgttgaa catgttacct tcttcatcta caataaaatt |
| 26101 | gttgatgagc ctgaagaaca tgtccaaatt cacacaatcg acggttcatc cggagttgtt |
| 26161 | aatccagtaa tggaaccaat ttatgatgaa ccgacgacga ctactagcgt gcctttgtaa |
| 26221 | gcacaagctg atgagtacga acttatgtac tcattcgttt cggaagagac aggtacgtta |
| 26281 | atagttaata gcgtacttct ttttcttgct ttcgtggtat tcttgctagt tacactagcc |
| 26341 | atccttactg cgcttcgatt gtgtgcgtac tgctgcaata ttgttaacgt gagtcttgta |
| 26401 | aaaccttctt tttacgttta ctctcgtgtt aaaaatctga attcttctag agttcctgat |
| 26461 | cttctggtct aaacgaacta aatattatat tagtttttct gtttggaact ttaattttag |
| 26521 | ccatggcaga ttccaacggt actattaccg ttgaagagct taaaaagctc cttgaacaat |
| 26581 | ggaacctagt aataggtttc ctattcctta catggatttg tcttctacaa tttgcctatg |
| 26641 | ccaacaggaa taggtttttg tatataatta agttaatttt cctctggctg ttatggccag |
| 26701 | taactttagc ttgttttgtg cttgctgctg tttacagaat aaattggatc accggtggaa |
| 26761 | ttgctatcgc aatggcttgt cttgtaggct tgatgtggct cagctacttc attgcttctt |
| 26821 | tcagactgtt tgcgcgtacg cgttccatgt ggtcattcaa tccagaaact aacattcttc |
| 26881 | tcaacgtgcc actccatggc actattctga ccagaccgct tctagaaagt gaactcgtaa |
| 26941 | tcggagctgt gatccttcgt ggacatcttc gtattgctgg acaccatcta ggacgctgtg |
| 27001 | acatcaagga cctgcctaaa gaaatcactg ttgctacatc acgaacgctt tcttattaca |
| 27061 | aattgggagc ttcgcagcgt gtagcaggtg actcaggttt tgctgcatac agtcgctaca |
| 27121 | ggattggcaa ctataaatta aacacagacc attccagtag cagtgacaat attgctttgc |
| 27181 | ttgtacagta agtgacaaca gatgtttcat ctcgttgact ttcaggttac tatagcagag |
| 27241 | atattactaa ttattatgag gacttttaaa gtttccattt ggaatcttga ttacatcata |
| 27301 | aacctcataa ttaaaaattt atctaagtca ctaactgaga ataaatattc tcaattagat |
| 27361 | gaagagcaac caatggagat tgattaaacg aacatgaaaa ttattctttt cttggcactg |
| 27421 | ataacactcg ctacttgtga gctttatcac taccaagagt gtgttagagg tacaacagta |
| 27481 | cttttaaaag aaccttgctc ttctggaaca tacgagggca attcaccatt tcatcctcta |
| 27541 | gctgataaca aatttgcact gacttgcttt agcactcaat ttgcttttgc ttgtcctgac |
| 27601 | ggcgtaaaac acgtctatca gttacgtgcc agatcagttt cacctaaact gttcatcaga |
| 27661 | caagaggaag ttcaagaact ttactctcca atttttctta ttgttgcggc aatagtgttt |
| 27721 | ataacacttt gcttcacact caaaagaaag acagaatgat tgaactttca ttaattgact |
| 27781 | tctatttgtg ctttttagcc tttctgctat tccttgtttt aattatgctt attatctttt |
| 27841 | ggttctcact tgaactgcaa gatcataatg aaacttgtca cgcctaaacg aacatgaaat |
| 27901 | ttcttgtttt cttaggaatc atcacaactg tagctgcatt tcaccaagaa tgtagtttac |
| 27961 | agtcatgtac tcaacatcaa ccatatgtag ttgatgaccc gtgtcctatt cacttctatt |
| 28021 | ctaaatggta tattagagta ggagctagaa aatcagcacc tttaattgaa ttgtgcgtgg |
| 28081 | atgaggctgg ttctaaatca cccattcagt acatcgatat cggtaattat acagtttcct |
| 28141 | gtttaccttt tacaattaat tgccaggaac ctaaattggg tagtcttgta gtgcgttgtt |
| 28201 | cgttctatga agacttttta gagtatcatg acgttcgtgt tgttttagat ttcatctaaa |
| 28261 | cgaacaaact aaaatgtctg ataatggacc ccaaaatcag cgaaatgcac cccgcattac |
| 28321 | gtttggtgga ccctcagatt caactggcag taaccagaat ggagaacgca gtggggcgcg |
| 28381 | atcaaaacaa cgtcggcccc aaggtttacc caataatact gcgtcttggt tcaccgctct |
| 28441 | cactcaacat ggcaaggaag accttaaatt ccctcgagga caaggcgttc caattaacac |
| 28501 | caatagcagt ccagatgacc aaattggcta ctaccgaaga gctaccagac gaattcgtgg |
| 28561 | tggtgacggt aaaatgaaag atctcagtcc aagatggtat ttctactacc taggaactgg |
| 28621 | gccagaagct ggacttccct atggtgctaa caaagacggc atcatatggg ttgcaactga |
| 28681 | gggagccttg aatacaccaa aagatcacat tggcacccgc aatcctgcta acaatgctgc |
| 28741 | aatcgtgcta caacttcctc aaggaacaac attgccaaaa ggcttctacg cagaagggag |
| 28801 | cagaggcggc agtcaagcct cttctcgttc ctcatcacgt agtcgcaaca gttcaagaaa |
| 28861 | ttcaactcca ggcagcagta ggggaacttc tcctgctaga atggctggca atggcggtga |
| 28921 | tgctgctctt gctttgctgc tgcttgacag attgaaccag cttgagagca aaatgtctgg |
| 28981 | taaaggccaa caacaacaag gccaaactgt cactaagaaa tctgctgctg aggcttctaa |
| 29041 | gaagcctcgg caaaaacgta ctgccactaa agcatacaat gtaacacaag ctttcggcag |
| 29101 | acgtggtcca gaacaaaccc aaggaaattt tggggaccag gaactaatca gacaaggaac |
| 29161 | tgattacaaa cattggccgc aaattgcaca atttgccccc agcgcttcag cgttcttcgg |
| 29221 | aatgtcgcgc attggcatgg aagtcacacc ttcgggaacg tggttgacct acacaggtgc |
| 29281 | catcaaattg gatgacaaag atccaaattt caaagatcaa gtcattttgc tgaataagca |
| 29341 | tattgacgca tacaaaacat tcccaccaac agagcctaaa aaggacaaaa agaagaaggc |
| 29401 | tgatgaaact caagccttac cgcagagaca gaagaaacag caaactgtga ctcttcttcc |
| 29461 | tgctgcagat ttggatgatt tctccaaaca attgcaacaa tccatgagca gtgctgactc |
| 29521 | aactcaggcc taaactcatg cagaccacac aaggcagatg ggctatataa acgttttcgc |
| 29581 | ttttccgttt acgatatata gtctactctt gtgcagaatg aattctcgta actacatagc |
| 29641 | acaagtagat gtagttaact ttaatctcac atagcaatct ttaatcagtg tgtaacatta |
| 29701 | gggaggactt gaaagagcca ccacattttc accgaggcca cgcggagtac gatcgagtgt |
| 29761 | acagtgaaca atgctaggga gagctgccta tatggaagag ccctaatgtg taaaattaat |
| 29821 | tttagtagtg ctatccccat gtgattttaa tagcttctta ggagaatgac aaaaaaaaaa |
| 29881 | aaaaaaaaaa aaaaaaaaaa aaa |

Start (atg) and stop codons (taa) are shown in bold type.

The membrane (M) protein is an integrity component of the viral membrane. The nucleocapsid (N) protein binds to the viral RNA and supports the nucleocapsid formation, assisting in virus budding, RNA replication, and mRNA replication. The envelope (E) protein is the least understood for its mechanism of action and structure, but seemingly plays roles in viral assembly, release, and pathogenesis.

### COVID-19 Vaccine Candidates

A vaccine is a biological preparation that provides active acquired immunity to a particular infectious disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. There are over 200 vaccine candidates for COVID-19 being pursued globally and these fall into several strategies:
1) Protein-based vaccines that generate target antigens in vitro such as inactivated virus vaccines, virus-like particles and protein subunit vaccines;
2) Gene-based vaccines that deliver genes encoding viral antigens to host cells for in vivo production such as virus-vectored vaccines,
3) DNA vaccines;
4) mRNA vaccines;
5) Combination of both protein-based and gene-based approaches to produce protein antigen or antigens both in vitro and in vivo, typically represented by live-attenuated virus vaccines;
   Cell-based approaches that use antigen-presenting cells (APC) such as dendritic cells (DC).

### SARS-CoV-2 Vaccine Epitopes

S protein is the main protein used as a target in COVID-19 vaccines. The S protein of the virus binds to the angiotensin-converting enzyme 2 (ACE2) receptor on the host cell surface, accompanied by being further primed by transmembrane protease serine (TMPRSS2). TMPRSS2 cleaves the S protein into two subunits, S1 and S2, during viral entry into the host cell via membrane fusion. ACE2 expression is ubiquitous in the nasal epithelium, lung, heart, kidney, and intestine, but it is rarely expressed in immune cells. Recent studies have shown that there are other receptors involved in viral entry in different cell types. As in the case of SARS-CoV, CD-147 on the epithelial cells is found to be a receptor for SARS-CoV-2 as well. CD26 (dipeptidyl peptidase 4, DPP4), originally discovered during the cellular entry of MERS-CoV, has also recently emerged as a potential receptor for SARS-CoV-2 and structural analysis showed SARS-CoV-2 S protein interaction with CD26 of the host cells. The critical role that the S protein plays in viral entry makes it an attractive target for COVID-19 vaccines.

The S1 subunit of the S protein contains the profusion-state of the receptor binding domain (RBD) responsible for binding to ACE2, while the S2 subunit contains the cleavage site that is critical for the fusion of viral and cellular membranes. Computational analyses and knowledge previously gained from SARS-CoV and MERS-CoV identified the full-length S protein, S1, RBD, and S2 subunit proteins to be key epitopes for inducing neutralizing antibodies. While structurally similar, the SARS-CoV-2 S protein has shown 20 times higher binding affinity to host cells than SARS-CoV S protein, explaining the high transmission rate of COVID-19. The S protein in both SARS-CoV and SARS-CoV-2 additionally induces the fusion between infected and non-infected cells, allowing for direct viral spread between cells while avoiding virus-neutralizing antibodies. The possibility of utilizing multiple neutralizing epitopes makes the S protein the most popular target for vaccination. In particular, the S1 epitope containing both the N-terminal binding domain (NTD) and RBD has been used in vaccine development, and especially the antibodies against the RBD domain have previously demonstrated to prevent infections by SARS-CoV and MERS-CoV.

The N protein is the most abundant protein among coronaviruses with a high level of conservancy. While patients have shown to develop antibodies against the N protein, its use in vaccination remains controversial. Some studies demonstrated strong N-specific humoral and cellular immune responses, while others showed insignificant contribution of the N protein to production of neutralizing antibodies.

Immunization with the M protein, a major protein on the surface of SARS-CoV-2, elicited efficient neutralizing antibodies in SARS patients. Structural analysis of the transmembrane portion of the M protein showed a T cell epitope cluster that enables the induction of strong cellular immune response against SARS-CoV, and it could also be a useful antigen in the development of SARS-CoV-2 vaccine. As compared to the S, N, and M proteins, E proteins of SARS-CoV-2 are not promising for vaccination as their structure low quantity is unlikely to induce an immune response.

### Challenges for Current COVID-19 Vaccines

Major hurdles in COVID-19 vaccine development include difficulty in validating and targeting the appropriate vaccine platform technologies, failure of generating long-term immunity, and inability to calm the cytokine storm. In addition to conventional vaccine forms of inactivated or live attenuated viruses, viral vectors, and subunit vaccines, emerging vaccine approaches using nanotechnology are highly adaptable and contribute to accelerated vaccine development. However, most of these platforms have not been licensed for use in humans yet, leading to questions of long-term safety as well as the degree to which they can induce strong and long-term immunity.

Electroporation and Delivery

In the past, platforms based on nucleic acids such as DNA and RNA have not resulted in a successful vaccine for human diseases and lipid nanoparticles are temperature-sensitive which may pose difficulties for scaling up production. Moreover, DNA vaccines are reliant on electroporation or an injector delivery device for vaccine administration which is problematic. Although electroporation (which is critical to generate an increased immune response) is considered to be a safe procedure, it can complicate vaccine delivery. Pre-existing immunity to adenoviruses is also a concern, particularly for those vaccine candidates utilizing human adenoviruses as it may result in a reduced immune response to the vaccine.

“S-Only” Vaccines

An additional key concern is relying on the “S-only” [vaccines targeting only the Spike (S) protein) vaccines], as mutations have been detected in the spike (S) protein of SARSCoV-2 and many candidate vaccines may need to be redesigned and tested. Mutations of the virus can result in vaccines having limited effectiveness against it. Historically, an ideal vaccine would be composed of an antigen or multiple antigens, adjuvant(s), and a delivery platform that can specifically be effective against the target infection, safe to a broad range of populations, and capable of inducing long-term immunity. Multiple coronavirus variants are circulating globally and three variants in particular that have mutations in the S protein are currently of significant concern as they appear to spread more easily and may affect the efficacy of approved vaccines. These variants are the UK “Kent” variant B.1.1.7, the South Africa variant B.1.351 and the Brazil variant P.1. Compared with the sequence shown above (SEQ ID NO: 1-S protein sequence), these variants have the following mutations: N501Y in B.1.1.7; K417N, E484K, and N501Y in B.1.351; and K417T, E484K, and N501Y in P.1 (Zhou D., Evidence of escape of SARS-CoV-2 variant B.1.351 from natural and vaccine-indice sera. Cell. 2021. 189:1-14). The appearance of these variants makes it likely that vaccines that target single S epitopes will need to be continually redesigned.

### Dendritic Cells (DCs)

Dendritic cells (DCs) are uniquely able to initiate primary immune responses. Because of their critical role in orchestrating the immune response, ex vivo DCs have been applied in vaccines. This approach involves direct ex vivo loading of antigens into autologous-derived DCs with an efficient DC stimulation through a “maturation cocktail”, which typically consists of a combination of pro-inflammatory cytokines and Toll-like receptor agonists. Besides targeting DC receptors, the ex vivo approach provides the possibility of applying a wide spectrum of more efficient antigen loading methods that cannot be applied in vivo. Ex vivo strategies of antigen loading to DCs include direct loading of proteins or peptides. Moreover, the transduction of DCs with viral vectors and mRNA, which encode antigens, could be applied. According to the invention, coronavirus-specific DCs are generated at a large scale in closed systems, yielding sufficient numbers of cells for clinical application.

In addition to conventional mRNA molecules, synthetic mRNAs that are expressed more rapidly are used in order to achieve more rapid in vivo responses. For example U.S. Pat. No. 9,657,282B2 (Factor Bio). Alternatively, DNA-encoding antigens or SARS-CoV-2 proteins or peptides are delivered to autologous or allogeneic DCs. Moreover, ‘TriMix’ mRNAs can be delivered in order to enhance DC functionality.

DCs are engineered to express proteins that enhance DC functionality. For example, the Soluble NSF attachment protein (SNAP) Receptor (SNARE) protein Vesicle-trafficking protein (SEC22B; human nucleic acid sequence GenBank Ref No: NM_004892.6 and human protein sequence GenBank Ref No: NP_004883.3) reduces antigen degradation by DCs. Delivery of SEC22b-encoding DNA or mRNA could thus enhance DC functionality.

Human SEC22b amino acid sequence GenBank Accession Number: NP_004883.3 (SEQ ID NO: 4) is provided below.

| | |
|---|---|
| 1 | mvlltmiarv adglplaasm qedeqsgrdl qqyqsqakql frklneqspt rctleagamt |
| 61 | fhyiieqgvc ylvlceaafp kklafayled lhsefdeqhg kkvptvsrpy sfiefdtfiq |
| 121 | ktkklyidsr arrnlgsint elqdvqrimv anieevlqrg ealsaldska nnlsslskky |
| 181 | rqdakylnmr styaklaava vffimlivyv rfwwl |

Exemplary landmark residues, domains, and fragments of SEC22b include, but are not limited to residues 1-13 (Signal sequence), residues 195-215 (transmembrane region). A fragment of an SEC22b protein is less than the length of the full length protein, e.g., a fragment is at least 3, 4, 5, 6, 7, 8, 9, 10, 20, 30, 40, 50, 100, 200 or more residues in length, but less than e.g., 215 residues in the case of SEC22b above.

Human SEC22b nucleic acid sequence is provided below with the start and stop codons bold and underlined. The GenBank Accession Number for the nucleic acid sequence is NM_004892.6 (SEQ ID NO: 5).

| | |
|---|---|
| 1 | acctcagcgg gaagcggaga cgcaagcagc tggatctccg gtaactgaga catagggtat |
| 61 | aactgttgtc gcggcggagg aagtgaggac ggcgccaagg gccttccggg ccagtgttgg |
| 121 | atccctgtag tttgtgaaga tggtgttgct aacaatgatc gcccgagtgg cggacgggct |
| 181 | cccgctggcc gcctcgatgc aggaggacga acagtctggc cgggaccttc aacaatatca |
| 241 | gagtcaggct aagcaactct ttcgaaagtt gaatgaacag tcccctacca gatgtacctt |
| 301 | ggaagcagga gccatgactt ttcactacat tattgagcag ggggtgtgtt atttggtttt |
| 361 | atgtgaagct gccttcccta agaagttggc ttttgcctac ctagaagatt tgcactcaga |
| 421 | atttgatgaa cagcatggaa agaaggtgcc cactgtgtcc cgaccctatt cctttattga |
| 481 | atttgatact ttcattcaga aaaccaagaa gctctacatt gacagtcgtg ctcgaagaaa |
| 541 | tctaggctcc atcaacactg aattgcaaga tgtgcagagg atcatggtgg ccaatattga |
| 601 | agaagtgtta caacgaggag aagcactctc agcattggat tcaaaggcta acaatttgtc |
| 661 | cagtctgtcc aagaaatacc gccaggatgc gaagtacttg aacatgcgtt ccacttatgc |
| 721 | caaacttgca gcagtagctg tatttttcat catgttaata gtgtatgtcc gattctggtg |
| 781 | gctgtgaaat aatgaataca gtcactggta agggagaacc tagaacccag taggtgtata |
| 841 | ttttcaggaa actgagctca cagagatgtg tattagaatc caagtggaac ttctgcctct |
| 901 | aaagaccttg caagaaaaga gatgccctga aaatgaaagg ttgcacctca tttaatgaag |
| 961 | cttaacccta tgtagaaagt ctctttcggg ggcagaggct ttctctgggt gccaagccat |
| 1021 | atatattagg gaatagtaga ttgttaattt cgttttttcc ctcccagtgc attttaaaaa |
| 1081 | cagcactggc tggggcattc tcattctctg atggagccat caatgagatt taacttagtc |
| 1141 | aacctgtgct agcaacattc tgaaattcct tcaaagaagg cagtcctttg ggaaggtgtt |
| 1201 | tttttttttt tttttttttt tgactctaat caacattcct tttgttggtg acatttgtga |
| 1261 | ttttcagtaa tctgagtttt tgatggcctt ttaaacaaga ctccagtatg tgaaggttaa |
| 1321 | ttgctgtgct ccacagatct tgtctattgg cccctgtaga aagttaacct ttgttgtttt |
| 1381 | ccttttataa tttgcttatt gcacaattgc tttagggtaa gtgaattata ttaagatgcc |
| 1441 | ttgaaattat agcactcctt gattaagaag ctaaaatgtt tctctcattt actccttaaa |
| 1501 | caaaagactt aaattagttt gggtcattat tacttttatt ttgcagcatt tggtttgtta |
| 1561 | ttagcgtaag agcaagtata ggatatggag aggcccctgg cttcatgaga acaaaggcag |
| 1621 | gcccaggtta taattacagc tttctcctgc cccttcttta ctttctctac cacagttttc |
| 1681 | tccactgttt gttttcctct tgccacaatt tgctaacatt taaaaaattt tcctgcaccc |
| 1741 | agtagtttca tatcctgtag acatcctctt aggacattct caaatttcaa aataaaaaat |
| 1801 | attcatctat gtagttaatt aaagttaaag tttttgcaga tcaactactc aaactactaa |
| 1861 | atacatttac ctgagaaaaa gtctctgaga gcacttcatt cctgttttag ttcgtgtaaa |
| 1921 | ttctctgaga atgttctgga gatagataac tcatttacag tggtttctat taactaatta |
| 1981 | aagtacccat gattttttcc ttttctgctc agggatgatg gagatttcct tttaccttct |
| 2041 | gaggtagaat tttttaatgg ggaaaatagg ccttttaaat attattgcca gggtctgcaa |
| 2101 | tataacttaa aattcctgta catactgcaa atatttcttt aaattgcaca ggaaaatgag |
| 2161 | cgaacttttt atttcttaat atctttggca aaaaacttta accagtaagc aattttataa |
| 2221 | ccctgaggga tcatcaaaga tactatcctg attcctggta aggaaaaata tattatttcc |
| 2281 | ttataacaag gcaaggagaa atgctatttt attcctgata atttatataa ctagaataat |
| 2341 | ttttttcctt tcttttatgg acctaaatct gccaattggg aattttgtgc atgaaatatg |
| 2401 | aagttacttt ttatagataa tcagtgcttt taagtcccta aaaggctcct gctgaagtaa |
| 2461 | tgatgatgtt aataataaaa gcctttgaaa ggctgaaaac ctacatagtg gtaccatagt |
| 2521 | atttggagct tctataggag tggagagggg cagctcattg ttgagagttg catgctgcaa |
| 2581 | cctaatggtc agcaatgaaa taaatacttg tagaatgttc acttcagtgt gaagttttgt |
| 2641 | tatctagtta atttatatac atatatcctt tgtagataca tttctatcta atcttgttgg |
| 2701 | gctaattaag aaataagggg tggggtaatt gtcaacaaag ggagaagaaa gtggtttaag |
| 2761 | atcagggcag cagaaaaatt agagaacaag aatatcataa tatggctcct ggttttcttt |
| 2821 | ataagaggca gtgggaagat ctgactagat gaaatgtatc atcaaccaaa ctggcatcta |
| 2881 | aaatagaatg ggataaatac tgtatggggt tattggaggc atattaagaa aggacaccta |
| 2941 | atttattttg ggaagaagta tgttaagaga agactttcta gagaaggaga atggggcatt |
| 3001 | ctaggaagag tcaatggcat gtgcaaaggc atgaataaag acagtgaggc atattttgga |
| 3061 | aatgtaacag cttgattcag cttagcccat agggtaagca tagacaacag aggagacttg |
| 3121 | aggaatgaga actagatggg tacactatca taaagggact tgtctatcat gctgaggagt |
| 3181 | ttagaccatc ttaatggtag tggccaagga tggcatcaga tttatagttt caagtgatca |
| 3241 | taatattggc ataaaagata tattagggag gaagcctgaa gtagggagat gaaaataagg |
| 3301 | agccatcaaa ggcagaatga aacttaggca gatttcaagt gattttcaaa aatgttgtga |
| 3361 | tcagaggtac cagataataa ctattacata acactttctt tgttaggagc ttatttctca |
| 3421 | cactgaccaa agcttttgaa gtaagtactc tttacaccac tatataaata aaccttacaa |
| 3481 | aggattctgc tttgaggcat gagagagtta agtcatttgc ccaaagtcac agagttagaa |
| 3541 | agtaatagag ctaagatttg aacctaggca gtttggctcc agagtctgtg ctcttacgct |
| 3601 | atattaccac caagaggtca aataaatacc aaagaatgta ttttcgaatt taacaatgag |
| 3661 | gaacttaatc atacaggcag aagtaattcc agagcactgg agacagaagc cagattgcca |
| 3721 | tatgggttaa agagtgtgta aaccactagg aagtaaagac atagaactac tctcacaagt |
| 3781 | gcttttctgg ttattgtgac gctgaacttc atggcttgtt ttaattaaga catcttacaa |
| 3841 | gtgtcaaaat ttggaaatat ttggacactg tacactctgg ttatttaaat atctaacaat |
| 3901 | ggttcttgag cattttgaga aacctttgaa aatctgatga aaggtatgtg ccattactct |
| 3961 | agaaaaatgt tcctgtgtac atgcacatca agtatcacat actgtttcag gctgttcaaa |
| 4021 | gactacaaag tctgttcatg acctaatggt ccatgttccc tcagttaaga gctccagaga |
| 4081 | taaaggatgt ggaactcaaa ggtaagtacc cagagccttg aaaactccat ctgtgacttg |
| 4141 | gaagaattct acaatttgaa ttaactttgt ggagagagat atatttttga aaaattgtgt |
| 4201 | gtaccaaaaa aatttcatat caaataatat tttcctgtag tgcattcaag gatctggttc |
| 4261 | cacagcaaaa aattgttttg gtctcagttc ctcaaaatca catttaagga gcttgagatt |
| 4321 | tatattttct acttaataag tcttacaaaa gcaagttaag aaggaaaatg gacaatcatt |
| 4381 | tctgcacata tagggtttaa taaaacatgt ataataaaat atctcatatt ttaaatttcc |
| 4441 | accttattgg tagctttcat gacaaagggc tagggtgctg atggccatac aattaaggtt |
| 4501 | tttggttagt tagttagcag aactaactga ctcctacctg gtgggttttt cttttgtttg |
| 4561 | gttggttggt tggttggttt tttcccagat ggctcaggag gaaggtaaat agcagtcatt |
| 4621 | gtatgtgtga cagagtttga gatagaatga gcatattgaa tctcacatcc tattcttatt |
| 4681 | actgtcaggc agcgttgacc tagcagtata aaactatctg aagcaatgta gtcactcagt |
| 4741 | tctcataaag tttatttcaa gtactgtaac aattcatgtt tggattagaa aagtcactag |
| 4801 | aaatttgact tccatatagt aatctatact tttttctctc atttccttca ttttttgagc |
| 4861 | cgtaagtgta aggcattttg ctggtattat tacaatggtt atgaggagtt tctttgcttg |
| 4921 | cccaaggtca catagctagc aagttaaagt agattcaaat ccaggcctgc tagataccaa |
| 4981 | attattattt aagagtactt ttcactactc ctaaataatg acacagatac gtttgtctta |
| 5041 | cacatttcac tttattgtca agttattagt atgtttattt tcaaaagtta ttttttgcaa |
| 5101 | tttcttttta ttattccgta ctttttaaat ttacttcatt atcacgtctt cctttattct |
| 5161 | ttttaaatag tttttgcttt tgttattttg ttttcccttt tttactcttg gtttgtaata |
| 5221 | cctctttcct tatttgctcc tttctcattt gatctcaatg ttaatccaac tgttttccac |
| 5281 | atctgattca ctaaaatttt agcccttaaa aaaaaaattc ctgtttttcc tatctccttt |
| 5341 | tgtccattct cttctccttg cctcacttct tttatctttt tccattttac tttcattttt |
| 5401 | tgtttctcta gatgttgttt tgacatatga gttaatgtac tggtacaatt ttgcatctgt |
| 5461 | aaattagagc ttcagaatca actgagtgta tttattcttt atttttaggc ctaaatttat |
| 5521 | cttacctttt attgatttta taatatacta tactctttca ttttagtctg catatgttag |
| 5581 | ccaaagaaga tatgcccctg ttttaagaaa tctctgtaaa aaatgtcaag tgtgacaaga |
| 5641 | attcttcaag aaacaagctc ctctagtttg tcttctatat ttagagcttc aacagttacc |
| 5701 | tatattactg gtaactccca aatatacctt caaacttgtt ttttgggccc aagttttttg |
| 5761 | cttcatatat atctgttttg aatatcccat aaataattgc atctaaagca tacctccact |
| 5821 | ccattgttct caaagataaa accaaacctg tgctgcttct tatatttcta gtatttaagc |
| 5881 | gtcacctgcc acccctttac ctgagctaca agtcacgcat tgcattagac tcctctgctt |
| 5941 | tcttctttca cctctaacta gactattaac caaaaatttt ttaatataac tttcaaaagg |
| 6001 | tattttatta catcatttcc atcctttatg tttgggttca agccctcatt aactttaaca |
| 6061 | tggattcttg gagtaccctc cttactgatt ttgttacaca tgtccctctt ttagtcagta |
| 6121 | ctaccatgat aataccatgg ataataattt tttcattttt atttttagtc ttgctctgtc |
| 6181 | gccgaggcta gagtgcagtg gcgcgatctc agctcactgc aacctccacc tcccgggttc |
| 6241 | aagtgattct cctgcctcag cctactgagt agctgggatt acaggcacct gccacagctc |
| 6301 | ctggctaatt tttgtatttt tagtagagat ggagtttcac cgtcttgaac tcctgacctc |
| 6361 | atgatccacc ctcctcggcc tcccaaagtg ctgggattac aggcatgggc cactgcgccc |
| 6421 | ggccaataat ttttgtgtgt gtgtgtgcta atcatactac attttcttta gaataaaaga |
| 6481 | tcacatactt gtttgccatt cgcagtctgg ccccattgtg ccattctaga cttacctcct |
| 6541 | gccactcccc accagctttg ttttgtctta gccacacaaa ataatctagc gtctctaacc |
| 6601 | agtcaaacat tttaccttgt gccttggctc actctgtgcc ttttctccag aatatctttc |
| 6661 | tgtgtacttt tctcccatcc ttttaccttt aaacctgctg ctatggtttg catgttgttt |
| 6721 | ggcccctcca aaactcatgt tgtagttcaa ttgccaatgt aatagtgttg ggagatggta |
| 6781 | cttttaagag gtaattaggt tgctaagatg gattaacatc tttctcttga cactgagact |
| 6841 | gggttctcct gggaatggtt agttcccaag agagtgagtt gttataaaac aatgctgcct |
| 6901 | cttctatttt gcgctttttg tttgcac |

Another example is expression of IL-12 or CXCL9 to enhance T cell activation by DCs. Another example, induction of CD40L expression via mRNA is useful as a maturation tool in some DC vaccines.

The methods described herein provide that proteins can be downregulated in DCs to enhance DC functionality. For example, YTH N6-Methyladenosine RNA Binding Protein 1 (YTHDF1) promotes antigen degradation. The SOLUPORE™ system of molecules can downregulate expression of YTHDF1, such as siRNA or gene editing systems such as CRISPR Cas9, could thus enhance DC functionality. Another example is knockdown of PD-L1 and PD-L2 which are used to improve T cell activation by DCs.

The functionally closed SOLUPORE™ system is deployed to effect needle-needle near-patient cell engineering of a vaccine-size dose of engineered cells.

As described herein, the SOLUPORE™ method is used to generate DC vaccines for other infectious diseases as well as non-infectious diseases, e.g., cancer. Moreover, as described herein, other delivery methods and/or vectors are used to generate DCs as outlined herein such as viral transduction, electroporation, lipofection, nanoparticles, magnetofection, cell squeezing, carrier molecules (e.g. Feldan shuttle technology), Poros technology, Ntrans technology, microinjection, microfluidic vortex shedding.

### Challenges in DC-Based Immunotherapies

Dendritic cells (DC) are uniquely able to initiate primary immune responses. Because of their critical role in orchestrating the immune response, ex vivo DC have been applied in vaccines. This approach involves direct ex vivo loading of antigens into autologous-derived DC with an efficient DC stimulation through a “maturation cocktail”, which typically consists of a combination of pro-inflammatory cytokines and Toll-like receptor agonists. Besides targeting DC receptors, the ex vivo approach provides the possibility of applying a wide spectrum of more efficient antigen loading methods that cannot be applied in vivo.

Ex vivo strategies of antigen loading to DC include direct loading of proteins or peptides. Moreover, the transduction of DC with viral vectors and mRNA, which encode antigens, could be applied. DCs can be generated at a large scale in closed systems, yielding sufficient numbers of cells for clinical application. For DC-based cancer vaccines, more broadly activated polyclonal antitumor immunity has been generated by loading the DC with multiple antigens or with tumor lysates to activate multiple CD8+ and CD4+ T cell clones. This approach is taken to more potently activate a polyclonal immune response, incorporating multiple adaptive and innate effectors in order to induce effective anti-tumor immunity and clinical response. If a similar approach was taken for COVD-19 vaccines where multiple epitopes were loaded into DC, it is possible that these vaccines would be more broad spectrum and the need to re-engineer vaccines regularly could be reduced.

In particular, as disclosed herein, DCs are loaded with combinations of coronavirus antigens in order to generate a broad spectrum response that is more likely to immunize the patient against multiple variants of the virus. In addition, the SOLUPORE™ technology is more gentle than other delivery technologies such as electroporation. This means that the DCs are less likely to be adversely affected by the delivery process and more likely to produce a robust response in T cells.

These drawbacks have thus far precluded wide-scale application of autologous DC-based vaccines (Cancer Immunol Immunother (2008) 57:1569-1577). An alternative approach is the use of allogeneic DC as vaccine vehicles. A major advantage of the use of alloDC (allogenic DC is the feasibility of preparing large clinical-grade batches that may be used for all patients, thus providing a more standardized DC vaccine in terms of phenotype and maturation status. In addition, bypassing the need for individually prepared vaccines represents a considerable logistic advantage. Although seemingly counter-intuitive, from a theoretical point of view alloDC-based vaccines might even induce a stronger vaccine-specific immune response than autoDC. Since an estimated 1-10% of the circulating T cell repertoire is directed against allo-antigens, alloDC may be expected to trigger a broadly reactive T-cell response with two possible advantages: (1) activation of tumor-reactive T-cells through fortuitous cross-reactivity and (perhaps more likely and more importantly:) (2) allo-antigens on the DC may provide T helper (Th) epitopes aiding in the optimal activation of Cytotoxic T Lymphocytes (CTL) against the tumor-related vaccine payload.

### Nucleic Acid Therapeutics

Nucleic acid therapeutics, both DNA- and RNA-based, have emerged as promising alternatives to conventional vaccine approaches. Early promising results did not lead to substantial investment in developing mRNA therapeutics, largely owing to concerns associated with mRNA instability, high innate immunogenicity and inefficient in vivo delivery. Instead, the field pursued DNA-based and protein-based therapeutic approaches. However, over the past decade, major technological innovation and research investment have enabled mRNA to become a promising therapeutic tool in the fields of vaccine development and protein replacement therapy (Nat Rev Drug Discov. 2018 April; 17(4): 261-279. ‘mRNA vaccines—a new era in vaccinology’).

The use of mRNA has several beneficial features over subunit, killed and live attenuated virus, as well as DNA-based vaccines. An important benefit is the safety of mRNA vaccines. mRNA is a non-infectious, non-integrating platform and there is no potential risk of infection or insertional mutagenesis. Additionally, mRNA is degraded by normal cellular processes, and its in vivo half-life can be regulated through the use of various modifications and delivery methods. The inherent immunogenicity of the mRNA can be down-modulated to further increase the safety profile. A second benefit of mRNA vaccines is their efficacy. Various modifications make mRNA more stable and highly translatable. mRNA is the minimal genetic vector; therefore, anti-vector immunity is avoided, and mRNA vaccines can be administered repeatedly. A third advantage of mRNA vaccines include their production. mRNA vaccines have the potential for rapid, inexpensive and scalable manufacturing, mainly owing to the high yields of in vitro transcription reactions.

There are two basic approaches for the delivery of mRNA vaccines that have been described to date. Direct injection of mRNA is comparatively rapid and cost-effective, but it does not yet allow precise and efficient cell-type-specific delivery. Alternatively, loading of mRNA into (dendritic cells) DC ex vivo, followed by re-infusion of the transfected cells. Ex vivo DC loading allows precise control of the cellular target, transfection efficiency and other cellular conditions. Although DC have been shown to internalize naked mRNA through a variety of endocytic pathways, ex vivo transfection efficiency is commonly increased using electroporation; in this case, mRNA molecules pass through membrane pores formed by a high-voltage pulse and directly enter the cytoplasm. This mRNA delivery approach has been favoured for its ability to generate high transfection efficiency without the need for a carrier molecule. DC that are loaded with mRNA ex vivo are then re-infused into the autologous vaccine recipient to initiate the immune response.

Compared to protein or peptide antigen loading, this approach is an attractive option due to the possibility of avoiding the need for identification of the patient's haplotype, as well as to avoid the requirement for antigen harvesting or production. It has been demonstrated that the transfection of mRNA encoding tumor-specific antigens into DC can induce an antigen-specific CD8+ and CD4+ T cell response (Cancers 2020, 12, 590). The following step of artificial DC maturation is required. Although this approach has been demonstrated to elicit a response, it is limited due to low transfection efficacy. Lipid-mediated mRNA transfection was proposed to enhance transfection efficacy. Nevertheless, it has been demonstrated that lipid-mediated mRNA transfection was not substantially effective compared to passive mRNA transfection. Moreover, this approach should be applied providently due to the potential that the lipids could be quite toxic. Electroporation has been shown to be the most effective method of mRNA transfection. Electroporation of DC has been successfully used in preclinical and clinical trials for treating cancer. Recent advances in the mRNA transfection approach are related to the so-called TriMix-formula. This approach involves mRNA transfection-based delivery of antigens alongside a combination of cluster of differentiation 40 ligand (CD40L), constitutively active toll-like receptor 4 (caTLR4), and cluster of differentiation 70 (CD70) encoding mRNAs. DC transfected with TriMix demonstrate an enhanced T cell activation potential. Vaccination with autologous TriMix-DC has been shown to be safe and capable of antigen-specific immune response activation. Antigen-encoding DNA delivery to DC has been also applied. Recently, several nanoparticle-based approaches to DNA delivery have been reported. Liposomes or gold nanoparticles functionalized with mannose-mimicking headgroups were used to deliver DNA plasmid to DC ex vivo. Although this approach demonstrates some efficacy, further study is required for translation to clinical studies.

While ex vivo DC loading is a heavily pursued method to generate cell-mediated immunity against cancer, development of infectious disease vaccines using this approach has been mainly limited to a therapeutic vaccine for HIV-1. HIV-1-infected individuals on highly active antiretroviral therapy were treated with autologous DC electroporated with mRNA encoding various HIV-1 antigens, and cellular immune responses were evaluated. This intervention proved to be safe and elicited antigen-specific CD4+ and CD8+ T cell responses, but no clinical benefit was observed. Another study in humans evaluated a CMV pp65 mRNA-loaded DC vaccination in healthy human volunteers and allogeneic stem cell recipients and reported induction or expansion of CMV-specific cellular immune responses. mRNA vaccines have elicited protective immunity against a variety of infectious agents in animal models and have therefore generated substantial optimism. However, recently published results from two clinical trials of mRNA vaccines for infectious diseases were somewhat modest, leading to more cautious expectations about the translation of preclinical success to the clinic.

Thus, the methods described herein provide for the use of the SOLUPORE™ system to engineer DCs for COVID-19 vaccinations.

### Advantages of Described Method

Compared to other loading/transfection methods, the SOLUPORE™ technology provides an efficient and gentle method for delivering cargos to cells ex vivo and enables retention of high levels of cell functionality. The importance of using immunocompetent DC in vaccination applications is well established (JExpMed, 194:769 (2001)) and the toxicity of lipofection and electroporation may reduce in vivo efficacy.

Another point of difference between the SOLUPORE™ technology and other delivery methods such as electroporation is that the SOLUPORE™ technology involves concentration of the cargo at the cell membrane. This may be important for DC-based vaccines because the nature of the immune response generated by DC depends heavily upon the mode of antigen uptake. Straightforward pulsing of DC, such as occurs with electroporation, is inferior in comparison to the targeting of antigens to specific receptors of DC (Baldin, A. et al. Cancers 2020, 12, p. 590). Antigens conjugated with receptor-specific antibodies or antigen modulation for specific recognition by DC receptors enhance antigen uptake and they are more likely to undergo cross-presentation. The concentration of cargo at the cell membrane that occurs during soluporation could therefore enhance the targeting of DC receptors thus enhance the processing and cross-presentation efficacy of DC.

It has been demonstrated that DC vaccines are capable of inducing a de novo immune response at a number of DC as low as 3-10×10e6 (Clin. Cancer Res. O. J. Am. Assoc. Cancer Res. 2016, 22, 2155-2166) which is well within the range of SOLUPORE™ technology.

The purpose of the present invention is to use the SOLUPORE™ technology to engineer DC for COVID-19 vaccinations. In this invention, the SOLUPORE™ technology will be used to engineer DC such that the DC (i) present coronavirus antigens and (ii) have enhanced functionality compared with other delivery methods such as incubation and electroporation. The SOLUPORE™ technology will be used to deliver mRNA encoding for SARS-CoV-2 antigens to dendritic cells ex vivo. In addition to conventional mRNA molecules, synthetic mRNAs that are expressed more rapidly can be used in order to achieve more rapid in vivo responses (see, e.g., U.S. Pat. No. 9,657,282 Factor Bio, incorporated herein by reference in its entirety. In particular, see col. 3: 1-16; col. 10: 48-col. 15:49 and col. 14: 14-48 of U.S. Pat. No. 9,657,282.

Alternatively, DNA-encoding antigens or SARS-CoV-2 proteins or peptides are delivered to DC. Additionally, ‘TriMix’ mRNAs can be delivered in order to enhance DC functionality. In another examples, DCs are engineered to express proteins that enhance DC functionality. For example, the SNARE protein SEC22B reduces antigen degradation by DC. Delivery of SEC22b-encoding DNA or mRNA could thus enhance DC functionality. Another example is expression of IL-12 or CXCL9 to enhance T cell activation by DC. Another example, induction of CD40L expression via mRNA is well established as a maturation tool in some DC vaccines.

In other embodiments, proteins can be downregulated in DCs to enhance DC functionality. For example, YTHDF1 promotes antigen degradation. Using SOLUPORE™ technology to deliver molecules that downregulate expression of YTHDF1, such as siRNA or gene editing systems such as CRISPR Cas9, could thus enhance DC functionality. Another example is knockdown of PD-L1 and PD-L2 which could improve T cell activation by DC. The PD-1/PDL axis is involved in inhibiting the function of T cells upon their engagement with PD-L1 expressing cells such as DCs. PD-1 is a co-inhibitory receptor that is inducibly expressed by T cells upon activation and can lead to T cell exhaustion. Therefore, knockdown of PD-L1 and PD-L2 could improve T cell activation by DC.

In addition, the functionally closed SOLUPORE™ system can be deployed to effect needle-needle near-patient cell engineering of a vaccine-size dose of engineered cells.

In other embodiments, the SOLUPORE™ technology is used as outlined above to generate DC vaccines for other infectious diseases as well as non-infectious diseases such as cancer. In further examples, other delivery methods and/or vectors are used to generate DC as outlined above such as viral transduction, electroporation, lipofection, nanoparticles, magnetofection, cell squeezing, carrier molecules (eg. Feldan shuttle technology), Poros technology, Ntrans technology, microinjection, or microfluidic vortex shedding.

### Advantages of Dendritic Cell Vaccines for Certain Cohorts

While the existing and imminent covid-19 vaccines are likely to be effective and safe in many people, there are certain cohorts for which concerns remain.

While serious adverse events have not been associated with the current vaccines, in many cases there has been substantial reactogenicity. Patients on cancer treatments have been excluded from Covid-19 vaccine trials thus far. Reactogenicity is not trivial for patients with cancer, for whom eg. fever carries a concerning differential (eg. infection, disease recurrence etc.). Dendritic cell vaccines tend to have fewer side effects compared with mRNA and DNA vaccines and so may be more suited to vaccinating cancer patients. Furthermore, given the concern about coronavirus variants, it is possible that at-risk cohorts, such as cancer patients, may need to receive repeated new vaccinations over time, similar to the annual ‘flu jab’. A dendritic cell vaccine that provides broad spectrum protection against multiple variants could reduce the number of re-vaccinations that are needed over time, thus reducing exposure to potentially harmful side effects.

There is also concern about Covid-19 vaccine uptake among minority ethnic groups, because vaccine uptake in previous vaccine programs over the past decade has been traditionally lower in these groups. In the UK in terms of general vaccinations, Black African and Black Caribbean groups are less likely to be vaccinated (50%) compared to White groups (70%). Furthermore, for new vaccines (post-2013), adults in minority ethnic groups were less likely to have received the vaccine compared to those in White groups (by 10-20%). During the Covid-19 pandemic, prior to vaccination roll-out in the UK, it has been shown that people of black and south Asian ethnic background have a greater risk of death from Covid than white people, with data suggesting black people have a fourfold higher risk of dying from Covid than white people. Given the likely need for repeat vaccinations for Covid-19 in order to tackle recurring variants, uptake of mRNA and DNA vaccines is likely to remain disproportionally low in these sub-populations. A dendritic cell vaccine that provides broad spectrum protection against multiple variants could reduce the number of re-vaccinations that are needed over time and so provide these minorities with greater protection.

An exemplary COVID-19 variant composite vaccine composition may be manufactured as follows. A method for engineering dendritic cells (DCs) to present a payload comprising one or more coronavirus antigens, e.g., a spike protein, e.g., a COVID-19 variant composite protein, coronavirus mRNA molecules, coronavirus synthetic mRNAs, or DNA-encoding coronavirus antigens peptides, is carried out by providing a population of patient-derived (allogeneic with respect to the eventual recipient) DCs and contacting the population of cells with a volume of an isotonic aqueous solution, the aqueous solution including the payload and an alcohol at greater than 2 percent (v/v) concentration (e.g., an isotonic solution comprising 106 mM KCl and 12% ethanol or other delivery solution variations as described herein). The DCs (from intended subject) are contacted with a mRNA encoding a protein comprising an amino acid sequence with at least 90% (91%, 92%, 93%, 94%, 95%, 96%, 97%, 98%, 98%, 99% or 100%) sequence identity to the amino acid sequence of SEQ ID NO: 30 e.g., the DCs are contacted with a mRNA encoding a protein comprising the amino acid sequence of SEQ ID NO: 30. The amino acid sequence of SEQ ID NO: 30 is shown below:

mfvflvllpl vssqcvnftt rtqlppaytn sftrgvyypd
kvfrssvlhs tqdlflpffs nvtwfhaihv sgtngtkrfd
npvlpfndgv yfasteksni irgwifgttl dsktqslliv
nnatnvvikv cefqfcndpf lgvyyhknnk swmesefrvy
ssannctfey vsqpflmdle gkqgnfknlr efvfknidgy
fkiyskhtpi nlvrdlpqgf saleplvdlp iginitrfqt
llalhisylt pggsssgwta gaaayyvgyl qprtfllkyn
engtitdavd caldplsetk ctlksftvek giyqtsnfrv
qptesivrfp nitnlcpfge vfnatrfasv yawnrkrisn
cvadysvlyn sasfstfkcy gvsptklndl cftnvyadsf
virgdevrqi apgqtgniad ynyklpddft gcviawnskn
ldskvggnyn yrfrlfrksn lkpferdist eiyqagntpc
ngvkgfncyf plqsygfqpt ygvgyqpyrv vvlsfellha
patvcgpkks tnlvknkcvn fnfngltgtg vltesnkkfl
pfqqfgrdia dttdavrdpq tleilditpc sfggvsvitp
gtntsnqvav lyqgvnctev pvaihadqlt ptwrvystgs
nvfqtragcl igaehvnnsy ecdipigagi casyqtptns
hrrarsvasq siiaytmslg vensvaysnn siaiptnfti
svtteilpvs mtktsvdctm yicgdstecs nlllqygsfc
tqlnraltgi aveqdkntqe vfaqvkqiyk tppikdfggf
nfsqilpdps kpskrsfied llfnkvtlad agfikqygdc
lgdiaardli caqkfngltv lpplltdemi aqytsallag
titsgwtfga gaalqipfam qmayrfngig vtqnvlyenq
klianqfnsa igkiqdslss tasalgklqd vvnqnaqaln
tlvkqlssnf gaissvlndi lsrldkveae vqidrlitgr
lqslqtyvtq qliraaeira sanlaatkms ecvlgqskrv
dfcgkgyhlm sfpqsaphgv vflhvtyvpa qeknfttapa
ichdgkahfp regvfvsngt hwfvtqrnfy epqiittdnt
fvsgncdvvi givnntvydp lqpeldsfke eldkyfknht
spdvdlgdis ginasvvniq keidrlneva knlneslidl
qelgkyeqyi kwpwyiwlgf iagliaivmv timlccmtsc
csclkgccsc gscckfdedd sepvlkgvkl hyt

This protein is a variant composite that contains the following spike protein mutations: L18F, R246I, D253G, K417N, N439K, L452R, Y453F, S477N, E484K, N501Y, D614G, Q677P, P681H, A701V. Alternatively, the protein is a variant composite that contains the following spike protein mutations: L18F, R246I, D253G, K417T, N439K, L452R, Y453F, S477N, E484K, N501Y, D614G, Q677H, P681H, A701V. The variant composite protein (containing a plurality of spike protein point mutations identified in COVID-19 variants) is encoded by the DNA sequence of SEQ ID NO:31, shown below:

atgtttgtgtttctggtgctgctgccgctggtgagcagccagtgcgtga
actttaccacccgcacccagctgccgccggcgtataccaacagctttac
ccgcggcgtgtattatccggataaagtgtttcgcagcagcgtgctgcat
agcacccaggatctgtttctgccgttttttagcaacgtgacctggtttc
atgcgattcatgtgagcggcaccaacggcaccaaacgctttgataaccc
ggtgctgccgtttaacgatggcgtgtattttgcgagcaccgaaaaaagc
aacattattcgcggctggatttttggcaccaccctggatagcaaaaccc
agagcctgctgattgtgaacaacgcgaccaacgtggtgattaaagtgtg
cgaatttcagttttgcaacgatccgtttctgggcgtgtattatcataaa
aacaacaaaagctggatggaaagcgaatttcgcgtgtatagcagcgcga
acaactgcacctttgaatatgtgagccagccgtttctgatggatctgga
aggcaaacagggcaactttaaaaacctgcgcgaatttgtgtttaaaaac
attgatggctattttaaaatttatagcaaacataccccgattaacctgg
tgcgcgatctgccgcagggctttagcgcgctggaaccgctggtggatct
gccgattggcattaacattacccgctttcagaccctgctggcgctgcat
attagctatctgaccccgggcggcagcagcagcggctggaccgcgggcg
cggcggcgtattatgtgggctatctgcagccgcgcacctttctgctgaa
atataacgaaaacggcaccattaccgatgcggtggattgcgcgctggat
ccgctgagcgaaaccaaatgcaccctgaaaagctttaccgtggaaaaag
gcatttatcagaccagcaactttcgcgtgcagccgaccgaaagcattgt
gcgctttccgaacattaccaacctgtgcccgtttggcgaagtgtttaac
gcgacccgctttgcgagcgtgtatgcgtggaaccgcaaacgcattagca
actgcgtggcggattatagcgtgctgtataacagcgcgagctttagcac
ctttaaatgctatggcgtgagcccgaccaaactgaacgatctgtgcttt
accaacgtgtatgcggatagctttgtgattcgcggcgatgaagtgcgcc
agattgcgccgggccagaccggcaacattgcggattataactataaact
gccggatgattttaccggctgcgtgattgcgtggaacagcaaaaacctg
gatagcaaagtgggcggcaactataactatcgctttcgcctgtttcgca
aaagcaacctgaaaccgtttgaacgcgatattagcaccgaaatttatca
ggcgggcaacaccccgtgcaacggcgtgaaaggctttaactgctatttt
ccgctgcagagctatggctttcagccgacctatggcgtgggctatcagc
cgtatcgcgtggtggtgctgagctttgaactgctgcatgcgccggcgac
cgtgtgcggcccgaaaaaaagcaccaacctggtgaaaaacaaatgcgtg
aactttaactttaacggcctgaccggcaccggcgtgctgaccgaaagca
acaaaaaatttctgccgtttcagcagtttggccgcgatattgcggatac
caccgatgcggtgcgcgatccgcagaccctggaaattctggatattacc
ccgtgcagctttggcggcgtgagcgtgattaccccgggcaccaacacca
gcaaccaggtggcggtgctgtatcagggcgtgaactgcaccgaagtgcc
ggtggcgattcatgcggatcagctgaccccgacctggcgcgtgtatagc
accggcagcaacgtgtttcagacccgcgcgggctgcctgattggcgcgg
aacatgtgaacaacagctatgaatgcgatattccgattggcgcgggcat
ttgcgcgagctatcagaccccgaccaacagccatcgccgcgcgcgcagc
gtggcgagccagagcattattgcgtataccatgagcctgggcgtggaaa
acagcgtggcgtatagcaacaacagcattgcgattccgaccaactttac
cattagcgtgaccaccgaaattctgccggtgagcatgaccaaaaccagc
gtggattgcaccatgtatatttgcggcgatagcaccgaatgcagcaacc
tgctgctgcagtatggcagcttttgcacccagctgaaccgcgcgctgac
cggcattgcggtggaacaggataaaaacacccaggaagtgtttgcgcag
gtgaaacagatttataaaaccccgccgattaaagattttggcggcttta
actttagccagattctgccggatccgagcaaaccgagcaaacgcagctt
tattgaagatctgctgtttaacaaagtgaccctggcggatgcgggcttt
attaaacagtatggcgattgcctgggcgatattgcggcgcgcgatctga
tttgcgcgcagaaatttaacggcctgaccgtgctgccgccgctgctgac
cgatgaaatgattgcgcagtataccagcgcgctgctggcgggcaccatt
accagcggctggacctttggcgcgggcgcggcgctgcagattccgtttg
cgatgcagatggcgtatcgctttaacggcattggcgtgacccagaacgt
gctgtatgaaaaccagaaactgattgcgaaccagtttaacagcgcgatt
ggcaaaattcaggatagcctgagcagcaccgcgagcgcgctgggcaaac
tgcaggatgtggtgaaccagaacgcgcaggcgctgaacaccctggtgaa
acagctgagcagcaactttggcgcgattagcagcgtgctgaacgatatt
ctgagccgcctggataaagtggaagcggaagtgcagattgatcgcctga
ttaccggccgcctgcagagcctgcagacctatgtgacccagcagctgat
tcgcgcggcggaaattcgcgcgagcgcgaacctggcggcgaccaaaatg
agcgaatgcgtgctgggccagagcaaacgcgtggatttttgcggcaaag
gctatcatctgatgagctttccgcagagcgcgccgcatggcgtggtgtt
tctgcatgtgacctatgtgccggcgcaggaaaaaaactttaccaccgcg
ccggcgatttgccatgatggcaaagcgcattttccgcgcgaaggcgtgt
ttgtgagcaacggcacccattggtttgtgacccagcgcaacttttatga
accgcagattattaccaccgataacacctttgtgagcggcaactgcgat
gtggtgattggcattgtgaacaacaccgtgtatgatccgctgcagccgg
aactggatagctttaaagaagaactggataaatattttaaaaaccatac
cagcccggatgtggatctgggcgatattagcggcattaacgcgagcgtg
gtgaacattcagaaagaaattgatcgcctgaacgaagtggcgaaaaacc
tgaacgaaagcctgattgatctgcaggaactgggcaaatatgaacagta
tattaaatggccgtggtatatttggctgggctttattgcgggcctgatt
gcgattgtgatggtgaccattatgctgtgctgcatgaccagctgctgca
gctgcctgaaaggctgctgcagctgcggcagctgctgcaaatttgatga
agatgatagcgaaccggtgctgaaaggcgtgaaactgcattatacc

For example, the mRNA delivered to the DCs comprises the ribonucleic acid sequence of SEO ID NO: 32, which is shown below:

AUGUUUGUGUUUCUGGUGCUGCUGCCGCUGGUGAGCAGCCAGUGCGUGA
ACUUUACCACCCGCACCCAGCUGCCGCCGGCGUAUACCAACAGCUUUAC
CCGCGGCGUGUAUUAUCCGGAUAAAGUGUUUCGCAGCAGCGUGCUGCAU
AGCACCCAGGAUCUGUUUCUGCCGUUUUUUAGCAACGUGACCUGGUUUC
AUGCGAUUCAUGUGAGCGGCACCAACGGCACCAAACGCUUUGAUAACCC
GGUGCUGCCGUUUAACGAUGGCGUGUAUUUUGCGAGCACCGAAAAAAGC
AACAUUAUUCGCGGCUGGAUUUUUGGCACCACCCUGGAUAGCAAAACCC
AGAGCCUGCUGAUUGUGAACAACGCGACCAACGUGGUGAUUAAAGUGUG
CGAAUUUCAGUUUUGCAACGAUCCGUUUCUGGGCGUGUAUUAUCAUAAA
AACAACAAAAGCUGGAUGGAAAGCGAAUUUCGCGUGUAUAGCAGCGCGA
ACAACUGCACCUUUGAAUAUGUGAGCCAGCCGUUUCUGAUGGAUCUGGA
AGGCAAACAGGGCAACUUUAAAAACCUGCGCGAAUUUGUGUUUAAAAAC
AUUGAUGGCUAUUUUAAAAUUUAUAGCAAACAUACCCCGAUUAACCUGG
UGCGCGAUCUGCCGCAGGGCUUUAGCGCGCUGGAACCGCUGGUGGAUCU
GCCGAUUGGCAUUAACAUUACCCGCUUUCAGACCCUGCUGGCGCUGCAU
AUUAGCUAUCUGACCCCGGGCGGCAGCAGCAGCGGCUGGACCGCGGGCG
CGGCGGCGUAUUAUGUGGGCUAUCUGCAGCCGCGCACCUUUCUGCUGAA
AUAUAACGAAAACGGCACCAUUACCGAUGCGGUGGAUUGCGCGCUGGAU
CCGCUGAGCGAAACCAAAUGCACCCUGAAAAGCUUUACCGUGGAAAAAG
GCAUUUAUCAGACCAGCAACUUUCGCGUGCAGCCGACCGAAAGCAUUGU
GCGCUUUCCGAACAUUACCAACCUGUGCCCGUUUGGCGAAGUGUUUAAC
GCGACCCGCUUUGCGAGCGUGUAUGCGUGGAACCGCAAACGCAUUAGCA
ACUGCGUGGCGGAUUAUAGCGUGCUGUAUAACAGCGCGAGCUUUAGCAC
CUUUAAAUGCUAUGGCGUGAGCCCGACCAAACUGAACGAUCUGUGCUUU
ACCAACGUGUAUGCGGAUAGCUUUGUGAUUCGCGGCGAUGAAGUGCGCC
AGAUUGCGCCGGGCCAGACCGGCAACAUUGCGGAUUAUAACUAUAAACU
GCCGGAUGAUUUUACCGGCUGCGUGAUUGCGUGGAACAGCAAAAACCUG
GAUAGCAAAGUGGGCGGCAACUAUAACUAUCGCUUUCGCCUGUUUCGCA
AAAGCAACCUGAAACCGUUUGAACGCGAUAUUAGCACCGAAAUUUAUCA
GGCGGGCAACACCCCGUGCAACGGCGUGAAAGGCUUUAACUGCUAUUUU
CCGCUGCAGAGCUAUGGCUUUCAGCCGACCUAUGGCGUGGGCUAUCAGC
CGUAUCGCGUGGUGGUGCUGAGCUUUGAACUGCUGCAUGCGCCGGCGAC
CGUGUGCGGCCCGAAAAAAAGCACCAACCUGGUGAAAAACAAAUGCGUG
AACUUUAACUUUAACGGCCUGACCGGCACCGGCGUGCUGACCGAAAGCA
ACAAAAAAUUUCUGCCGUUUCAGCAGUUUGGCCGCGAUAUUGCGGAUAC
CACCGAUGCGGUGCGCGAUCCGCAGACCCUGGAAAUUCUGGAUAUUACC
CCGUGCAGCUUUGGCGGCGUGAGCGUGAUUACCCCGGGCACCAACACCA
GCAACCAGGUGGCGGUGCUGUAUCAGGGCGUGAACUGCACCGAAGUGCC
GGUGGCGAUUCAUGCGGAUCAGCUGACCCCGACCUGGCGCGUGUAUAGC
ACCGGCAGCAACGUGUUUCAGACCCGCGCGGGCUGCCUGAUUGGCGCGG
AACAUGUGAACAACAGCUAUGAAUGCGAUAUUCCGAUUGGCGCGGGCAU
UUGCGCGAGCUAUCAGACCCCGACCAACAGCCAUCGCCGCGCGCGCAGC
GUGGCGAGCCAGAGCAUUAUUGCGUAUACCAUGAGCCUGGGCGUGGAAA
ACAGCGUGGCGUAUAGCAACAACAGCAUUGCGAUUCCGACCAACUUUAC
CAUUAGCGUGACCACCGAAAUUCUGCCGGUGAGCAUGACCAAAACCAGC
GUGGAUUGCACCAUGUAUAUUUGCGGCGAUAGCACCGAAUGCAGCAACC
UGCUGCUGCAGUAUGGCAGCUUUUGCACCCAGCUGAACCGCGCGCUGAC
CGGCAUUGCGGUGGAACAGGAUAAAAACACCCAGGAAGUGUUUGCGCAG
GUGAAACAGAUUUAUAAAACCCCGCCGAUUAAAGAUUUUGGCGGCUUUA
ACUUUAGCCAGAUUCUGCCGGAUCCGAGCAAACCGAGCAAACGCAGCUU
UAUUGAAGAUCUGCUGUUUAACAAAGUGACCCUGGCGGAUGCGGGCUUU
AUUAAACAGUAUGGCGAUUGCCUGGGCGAUAUUGCGGCGCGCGAUCUGA
UUUGCGCGCAGAAAUUUAACGGCCUGACCGUGCUGCCGCCGCUGCUGAC
CGAUGAAAUGAUUGCGCAGUAUACCAGCGCGCUGCUGGCGGGCACCAUU
ACCAGCGGCUGGACCUUUGGCGCGGGCGCGGCGCUGCAGAUUCCGUUUG
CGAUGCAGAUGGCGUAUCGCUUUAACGGCAUUGGCGUGACCCAGAACGU
GCUGUAUGAAAACCAGAAACUGAUUGCGAACCAGUUUAACAGCGCGAUU
GGCAAAAUUCAGGAUAGCCUGAGCAGCACCGCGAGCGCGCUGGGCAAAC
UGCAGGAUGUGGUGAACCAGAACGCGCAGGCGCUGAACACCCUGGUGAA
ACAGCUGAGCAGCAACUUUGGCGCGAUUAGCAGCGUGCUGAACGAUAUU
CUGAGCCGCCUGGAUAAAGUGGAAGCGGAAGUGCAGAUUGAUCGCCUGA
UUACCGGCCGCCUGCAGAGCCUGCAGACCUAUGUGACCCAGCAGCUGAU
UCGCGCGGCGGAAAUUCGCGCGAGCGCGAACCUGGCGGCGACCAAAAUG
AGCGAAUGCGUGCUGGGCCAGAGCAAACGCGUGGAUUUUUGCGGCAAAG
GCUAUCAUCUGAUGAGCUUUCCGCAGAGCGCGCCGCAUGGCGUGGUGUU
UCUGCAUGUGACCUAUGUGCCGGCGCAGGAAAAAAACUUUACCACCGCG
CCGGCGAUUUGCCAUGAUGGCAAAGCGCAUUUUCCGCGCGAAGGCGUGU
UUGUGAGCAACGGCACCCAUUGGUUUGUGACCCAGCGCAACUUUUAUGA
ACCGCAGAUUAUUACCACCGAUAACACCUUUGUGAGCGGCAACUGCGAU
GUGGUGAUUGGCAUUGUGAACAACACCGUGUAUGAUCCGCUGCAGCCGG
AACUGGAUAGCUUUAAAGAAGAACUGGAUAAAUAUUUUAAAAACCAUAC
CAGCCCGGAUGUGGAUCUGGGCGAUAUUAGCGGCAUUAACGCGAGCGUG
GUGAACAUUCAGAAAGAAAUUGAUCGCCUGAACGAAGUGGCGAAAAACC
UGAACGAAAGCCUGAUUGAUCUGCAGGAACUGGGCAAAUAUGAACAGUA
UAUUAAAUGGCCGUGGUAUAUUUGGCUGGGCUUUAUUGCGGGCCUGAUU
GCGAUUGUGAUGGUGACCAUUAUGCUGUGCUGCAUGACCAGCUGCUGCA
GCUGCCUGAAAGGCUGCUGCAGCUGCGGCAGCUGCUGCAAAUUUGAUGA
AGAUGAUAGCGAACCGGUGCUGAAAGGCGUGAAACUGCAUUAUACC

Also within the invention is a dendritic cell (or population of dendritic cells) comprising a protein comprising an amino acid sequence with at least 90% (91%, 92%, 93%, 94%, 95%, 96%, 97%, 98%, 98%, 99% or 100%) sequence identity to the amino acid sequence of SEQ ID NO: 30. For example, the dendritic cell comprises a protein comprising the amino acid sequence of SEQ ID NO: 30.

The DCs (from intended subject) are contacted with a DNA comprising a sequence with at least 90% (91%, 92%, 93%, 94%, 95%, 96%, 97%, 98%, 98%, 99% or 100%) sequence identity to the DNA sequence of SEQ ID NO: 31.

The DCs (from intended subject) are contacted with a mRNA comprising a sequence with at least 90% (91%, 92%, 93%, 94%, 95%, 96%, 97%, 98%, 98%, 99% or 100%) sequence identity to the DNA sequence of SEQ ID NO: 32.

A vaccine comprising such dendritic cells is associated with numerous advantages compared to first generation mRNA vaccines currently in use. Such advantages are described above.

### Methods of Preparation of Coronavirus-Specific Dendritic Cells

The agents (e.g., coronavirus antigens, conventional mRNA molecules, synthetic mRNAs, DNA-encoding antigens or SARS-CoV-2 proteins or peptides) are delivered into the cytoplasm of dendritic cells by contacting the cells with a solution containing a compound(s) to be delivered (e.g., e.g., coronavirus antigens, conventional mRNA molecules, synthetic mRNAs, DNA-encoding antigens or SARS-CoV-2 proteins or peptides) and an agent that reversibly permeates or dissolves a cell membrane. Preferably, the solution is delivered to the cells in the form of a spray, e.g., aqueous particles. (see, e.g., PCT/US2015/057247 and PCT/IB2016/001895, each of which are hereby incorporated in their entirety by reference). For example, the cells are coated with the spray but not soaked or submersed in the delivery compound-containing solution. Exemplary agents that permeate or dissolve a eukaryotic cell membrane include alcohols and detergents such as ethanol and Triton X-100, respectively. Other exemplary detergents, e.g., surfactants include polysorbate 20 (e.g., Tween 20), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), sodium dodecyl sulfate (SDS), and octyl glucoside.

An example of conditions to achieve a coating of a population of coated cells include delivery of a fine particle spray, e.g., the conditions exclude dropping or pipetting a bolus volume of solution on the cells such that a substantial population of the cells are soaked or submerged by the volume of fluid. Thus, the mist or spray comprises a ratio of volume of fluid to cell volume. Alternatively, the conditions comprise a ratio of volume of mist or spray to exposed cell area, e.g., area of cell membrane that is exposed when the cells exist as a confluent or substantially confluent layer on a substantially flat surface such as the bottom of a tissue culture vessel, e.g., a well of a tissue culture plate, e.g., a microtiter tissue culture plate.

“Cargo” or “payload” are terms used to describe a compound, or composition that is delivered via an aqueous solution across a cell plasma membrane and into the interior of a cell. For example, the cargo or payload may include coronavirus antigens, conventional mRNA molecules, synthetic mRNAs, DNA-encoding antigens or SARS-CoV-2 proteins or peptides.

In an aspect, delivering a payload across a plasma membrane of a cell includes providing a population of cells and contacting the population of cells with a volume of an aqueous solution. The aqueous solution includes the payload and an alcohol content greater than 5 percent concentration. In other examples, the aqueous solution includes the payload and an alcohol of less than 5 percent or less than 2 percent. In embodiments, the alcohol may be zero percent. The volume of the aqueous solution may be a function of exposed surface area of the population of cells, or may be a function of a number of cells in the population of cells.

In another aspect, a composition for delivering a payload across a plasma membrane of a cell includes an aqueous solution including the payload, an alcohol at greater than 5 percent concentration, greater than 46 mM salt, less than 121 mM sugar, and less than 19 mM buffering agent. For example, the alcohol, e.g., ethanol, concentration does not exceed 50%.

One or more of the following features can be included in any feasible combination. The volume of solution to be delivered to the cells is a plurality of units, e.g., a spray, e.g., a plurality of droplets on aqueous particles. The volume is described relative to an individual cell or relative to the exposed surface area of a confluent or substantially confluent (e.g., at least 75%, at least 80% confluent, e.g., 85%, 90%, 95%, 97%, 98%, 100%) cell population. For example, the volume can be between 6.0×10⁻⁷ microliter per cell and 7.4×10⁻⁴ microliter per cell. The volume is between 4.9×10⁻⁶ microliter per cell and 2.2×10⁻³ microliter per cell. The volume can be between 9.3×10⁻⁶ microliter per cell and 2.8×10⁻⁵ microliter per cell. The volume can be about 1.9×10⁻⁵ microliters per cell, and about is within 10 percent. The volume is between 6.0×10⁻⁷ microliter per cell and 2.2×10⁻³ microliter per cell. The volume can be between 2.6×10⁻⁹ microliter per square micrometer of exposed surface area and 1.1×10⁻⁶ microliter per square micrometer of exposed surface area. The volume can be between 5.3×10-8 microliter per square micrometer of exposed surface area and 1.6×10⁻⁷ microliter per square micrometer of exposed surface area. The volume can be about 1.1×10⁻⁷ microliter per square micrometer of exposed surface area. About can be within 10 percent.

Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel.

Contacting the population of cells with the volume of aqueous solution can be performed by gas propelling the aqueous solution to form a spray. The gas can include nitrogen, ambient air, or an inert gas. The spray can include discrete units of volume ranging in size from, 1 nm to 100 μm, e.g., 30-100 μm in diameter. The spray includes discrete units of volume with a diameter of about 30-50 μm. A total volume of aqueous solution of 20 μl can be delivered in a spray to a cell-occupied area of about 1.9 cm², e.g., one well of a 24-well culture plate. A total volume of aqueous solution of 10 μl is delivered to a cell-occupied area of about 0.95 cm², e.g., one well of a 48-well culture plate. Typically, the aqueous solution includes a payload to be delivered across a cell membrane and into cell, and the second volume is a buffer or culture medium that does not contain the payload. Alternatively, the second volume (buffer or media) can also contain payload. In some embodiments, the aqueous solution includes a payload and an alcohol, and the second volume does not contain alcohol (and optionally does not contain payload). The population of cells can be in contact with said aqueous solution for 0.1 10 minutes prior to adding a second volume of buffer or culture medium to submerse or suspend said population of cells. The buffer or culture medium can be phosphate buffered saline (PBS). The population of cells can be in contact with the aqueous solution for 2 seconds to 5 minutes prior to adding a second volume of buffer or culture medium to submerse or suspend the population of cells. The population of cells can be in contact with the aqueous solution, e.g., containing the payload, for 30 seconds to 2 minutes prior to adding a second volume of buffer or culture medium, e.g., without the payload, to submerse or suspend the population of cells. The population of cells can be in contact with a spray for about 1-2 minutes prior to adding the second volume of buffer or culture medium to submerse or suspend the population of cells. During the time between spraying of cells and addition of buffer or culture medium, the cells remain hydrated by the layer of moisture from the spray volume.

The aqueous solution can include an ethanol concentration of 5 to 30%. The aqueous solution can include one or more of 75 to 98% H₂O, 2 to 45% ethanol, 6 to 91 mM sucrose, 2 to 500 mM KCl, 2 to 35 mM ammonium acetate, and 1 to 14 mM (4-(2-hydroxyethyl)-1-piperazineethanesulfonic acid) (HEPES). For example, the delivery solution contains 106 mM KCl and 10-27% ethanol, e.g., 12% ethanol v/v.

The population of cells includes, for example, dendritic cells (DCs), which are antigen-presenting cells (also known as accessory cells) of the mammalian immune system. Their main function is to process antigen material and present it on the cell surface to the T cells of the immune system. They act as messengers between the innate and the adaptive immune systems.

The payload can include a small chemical molecule, a peptide or protein, or a nucleic acid. The small chemical molecule can be less than 1,000 Da. The chemical molecule can include MitoTracker® Red CMXRos, propidium iodide, methotrexate, and/or DAPI (4′,6-diamidino-2-phenylindole). The peptide can be about 5,000 Da. The peptide can include ecallantide under trade name Kalbitor, is a 60 amino acid polypeptide for the treatment of hereditary angioedema and in prevention of blood loss in cardiothoracic surgery), Liraglutide (marketed as the brand name Victoza, is used for the treatment of type II diabetes, and Saxenda for the treatment of obesity), and Icatibant (trade name Firazyer, a peptidomimetic for the treatment of acute attacks of hereditary angioedema). The small-interfering ribonucleic acid (siRNA) molecule can be about 20-25 base pairs in length, or can be about 10,000-15,000 Da. The siRNA molecule can reduces the expression of any gene product, e.g., knockdown of gene expression of clinically relevant target genes or of model genes, e.g., glyceraldehyde-3phosphate dehydrogenase (GAPDH) siRNA, GAPDH siRNA-FITC, cyclophilin B siRNA, and/or lamin siRNA. Protein therapeutics can include peptides, enzymes, structural proteins, receptors, cellular proteins, or circulating proteins, or fragments thereof. The protein or polypeptide be about 100-500,000 Da, e.g., 1,000-150,000 Da. The protein can include any therapeutic, diagnostic, or research protein or peptide, e.g., beta-lactoglobulin, ovalbumin, bovine serum albumin (BSA), and/or horseradish peroxidase. In other examples, the protein can include a cancer-specific apoptotic protein, e.g., Tumor necrosis factor-related apoptosis inducing protein (TRAIL).

An antibody is generally be about 150,000 Da in molecular mass. The antibody can include an anti-actin antibody, an anti-GAPDH antibody, an anti-Src antibody, an anti-Myc ab, and/or an anti-Raf antibody. The antibody can include a green fluorescent protein (GFP) plasmid, a GLuc plasmid and, and a BATEM plasmid. The DNA molecule can be greater than 5,000,000 Da. In some examples, the antibody can be a murine-derived monoclonal antibody, e.g., ibritumomab tiuxetin, muromomab-CD3, tositumomab, a human antibody, or a humanized mouse (or other species of origin) antibody. In other examples, the antibody can be a chimeric monoclonal antibody, e.g., abciximab, basiliximab, cetuximab, infliximab, or rituximab. In still other examples, the antibody can be a humanized monoclonal antibody, e.g., alemtuzamab, bevacizumab, certolizumab pegol, daclizumab, gentuzumab ozogamicin, trastuzumab, tocilizumab, ipilimumamb, or panitumumab. The antibody can comprise an antibody fragment, e.g., abatecept, aflibercept, alefacept, or etanercept. The invention encompasses not only an intact monoclonal antibody, but also an immunologically-active antibody fragment, e. g., a Fab or (Fab)2 fragment; an engineered single chain Fv molecule; or a chimeric molecule, e.g., an antibody which contains the binding specificity of one antibody, e.g., of murine origin, and the remaining portions of another antibody, e.g., of human origin.

The payload can include a therapeutic agent. For example, the cargo or payload may include coronavirus antigens, conventional mRNA molecules, synthetic mRNAs, DNA-encoding antigens or SARS-CoV-2 proteins or peptides. A therapeutic agent, e.g., a drug, or an active agent”, can mean any compound useful for therapeutic or diagnostic purposes, the term can be understood to mean any compound that is administered to a patient for the treatment of a condition. Accordingly, a therapeutic agent can include, proteins, peptides, antibodies, antibody fragments, and small molecules. Therapeutic agents described in U.S. Pat. No. 7,667,004 (incorporated herein by reference) can be used in the methods described herein. The therapeutic agent can include at least one of cisplatin, aspirin, statins (e.g., pitavastatin, atorvastatin, lovastatin, pravastatin, rosuvastatin, simvastatin, promazine HCl, chloropromazine HCl, thioridazine HCl, Polymyxin B sulfate, chloroxine, benfluorex HCl and phenazopyridine HCl), and fluoxetine. The payload can include a diagnostic agent. The diagnostic agent can include a detectable label or marker such as at least one of methylene blue, patent blue V, and indocyanine green. The payload can include a fluorescent molecule. The payload can include a detectable nanoparticle. The nanoparticle can include a quantum dot.

The population of non-adherent cells can be substantially confluent, such as greater than 75 percent confluent. Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel. The population of cells can form a monolayer of cells.

The alcohol can be selected from methanol, ethanol, isopropyl alcohol, butanol and benzyl alcohol. The salt can be selected from NaCl, KCl, Na₂HPO₄, KH₂PO₄, and C₂H₃O₂NH. In preferred embodiments, the salt is KCl. The sugar can include sucrose. The buffering agent can include 4-2-(hydroxyethyl)-1-piperazineethanesulfonic acid.

The present subject matter relates to a method for delivering molecules across a plasma membrane. The present subject matter finds utility in the field of intra-cellular delivery, and has application in, for example, delivery of molecular biological and pharmacological therapeutic agents to a target site, such as a cell, tissue, or organ. The method of the present subject matter comprises introducing the molecule to an aqueous composition to form a matrix; atomizing the matrix into a spray; and contacting the matrix with a plasma membrane.

This present subject matter relates to a composition for use in delivering molecules across a plasma membrane. The present subject matter finds utility in the field of intra-cellular delivery, and has application in, for example, delivery of molecular biological and pharmacological therapeutic agents to a target site, such as a cell, tissue, or organ. The composition of the present subject matter comprises an alcohol; a salt; a sugar; and/or a buffering agent.

In some implementations, demonstrated is a permeabilisation technique that facilitates intracellular delivery of molecules independent of the molecule and cell type. Nanoparticles, small molecules, nucleic acids, proteins and other molecules can be efficiently delivered into suspension cells or adherent cells in situ, including primary cells and stem cells, with low cell toxicity and the technique is compatible with high throughput and automated cell-based assays.

The example methods described herein include a payload, wherein the payload includes an alcohol. By the term “an alcohol” is meant a polyatomic organic compound including a hydroxyl (—OH) functional group attached to at least one carbon atom. The alcohol may be a monohydric alcohol and may include at least one carbon atom, for example methanol. The alcohol may include at least two carbon atoms (e.g. ethanol). In other aspects, the alcohol comprises at least three carbons (e.g. isopropyl alcohol). The alcohol may include at least four carbon atoms (e.g., butanol), or at least seven carbon atoms (e.g., benzyl alcohol). The example payload may include no more than 50% (v/v) of the alcohol, more preferably, the payload comprises 2-45% (v/v) of the alcohol, 5-40% of the alcohol, and 10-40% of the alcohol. The payload may include 20-30% (v/v) of the alcohol.

Most preferably, the payload delivery solution includes 25% (v/v) of the alcohol. Alternatively, the payload can include 2-8% (v/v) of the alcohol, or 2% of the alcohol. The alcohol may include ethanol and the payload comprises 5, 10, 20, 25, 30, and up to 40% or 50% (v/v) of ethanol, e.g., 27%. Example methods may include methanol as the alcohol, and the payload may include 5, 10, 20, 25, 30, or 40% (v/v) of the methanol. The payload may include 2-45% (v/v) of methanol, 20-30% (v/v), or 25% (v/v) methanol. Preferably, the payload includes 20-30% (v/v) of methanol. Further alternatively, the alcohol is butanol and the payload comprises 2, 4, or 8% (v/v) of the butanol.

In some aspects of the present subject matter, the payload is in an isotonic solution or buffer.

According to the present subject matter, the payload may include at least one salt. The salt may be selected from NaCl, KCl, Na₂HPO₄, C₂H₃O₂NH₄ and KH₂PO₄. For example, KCl concentration ranges from 2 mM to 500 mM. In some preferred embodiments, the concentration is greater than 100 mM, e.g., 106 mM.

According to example methods of the present subject matter, the payload may include a sugar (e.g., a sucrose, or a disaccharide). According to example methods, the payload comprises less than 121 mM sugar, 6-91 mM, or 26-39 mM sugar. Still further, the payload includes 32 mM sugar (e.g., sucrose). Optionally, the sugar is sucrose and the payload comprises 6.4, 12.8, 19.2, 25.6, 32, 64, 76.8, or 89.6 mM sucrose.

According to example methods of the present subject matter, the payload may include a buffering agent (e.g. a weak acid or a weak base). The buffering agent may include a zwitterion. According to example methods, the buffering agent is 4-(2-hydroxyethyl)-1-piperazineethanesulfonic acid. The payload may comprise less than 19 mM buffering agent (e.g., 1-15 mM, or 4-6 mM or 5 mM buffering agent). According to example methods, the buffering agent is 4-(2-hydroxyethyl)-1-piperazineethanesulfonic acid and the payload comprises 1, 2, 3, 4, 5, 10, 12, 14 mM 4-(2-hydroxyethyl)-1-piperazineethanesulfonic acid. Further preferably, the payload comprises 5 mM 4-(2-hydroxyethyl)-1-piperazineethanesulfonic acid.

According to example methods of the present subject matter, the payload includes ammonium acetate. The payload may include less than 46 mM ammonium acetate (e.g., between 2-35 mM, 10-15 mM, ore 12 mM ammonium acetate). The payload may include 2.4, 4.8, 7.2, 9.6, 12, 24, 28.8, or 33.6 mM ammonium acetate.

The volume of aqueous solution performed by gas propelling the aqueous solution may include compressed air (e.g. ambient air), other implementations may include inert gases, for example, helium, neon, and argon.

In certain aspects of the present subject matter, the population of cells may include dendritic cells (DCs).

In certain aspects of the present subject matter, the population of cells may be substantially confluent, and substantially may include greater than 75 percent confluent. In preferred implementations, the population of cells may form a single monolayer.

According to example methods, the payload to be delivered has an average molecular weight of up to 20,000,000 Da. In some examples, the payload to be delivered can have an average molecular weight of up to 2,000,000 Da. In some implementations, the payload to be delivered may have an average molecular weight of up to 150,000 Da. In further implementations, the payload to be delivered has an average molecular weight of up to 15,000 Da, 5,000 Da or 1,000 Da.

The payload to be delivered across the plasma membrane of a cell may include a small chemical molecule, a peptide or protein, a polysaccharide or a nucleic acid or a nanoparticle. A small chemical molecule may be less than 1,000 Da, peptides may have molecular weights about 5,000 Da, siRNA may have molecular weights around 15,000 Da, antibodies may have molecular weights of about 150,000 Da and DNA may have molecular weights of greater than or equal to 5,000,000 Da. In preferred embodiments, the payload comprises mRNA.

According to example methods, the payload includes 3.0-150.0 μM of a molecule to be delivered, more preferably, 6.6-150.0 μM molecule to be delivered (e.g. 3.0, 3.3, 6.6, or 150.0 μM molecule to be delivered). In some implementations, the payload to be delivered has an average molecular weight of up to 15,000 Da, and the payload includes 3.3 μM molecules to be delivered.

According to example methods, the payload to be delivered has an average molecular weight of up to 15,000 Da, and the payload includes 6.6 μM to be delivered. In some implementations, the payload to be delivered has an average molecular weight of up to 1,000 Da, and the payload includes 150.0 μM to be delivered.

According to further aspects of the present subject matter, a method for delivering molecules of more than one molecular weight across a plasma membrane is provided; the method including the steps of: introducing the molecules of more than one molecular weight to an aqueous solution; and contacting the aqueous solution with a plasma membrane.

In some implementations, the method includes introducing a first molecule having a first molecular weight and a second molecule having a second molecular weight to the payload, wherein the first and second molecules may have different molecular weights, or wherein, the first and second molecules may have the same molecular weights. According to example methods, the first and second molecules may be different molecules.

In some implementations, the payload to be delivered may include a therapeutic agent, or a diagnostic agent, including, for example, coronavirus antigens, conventional mRNA molecules, synthetic mRNAs, DNA-encoding antigens or SARS-CoV-2 proteins or peptides. Additionally, the therapeutic agent may include cisplatin, aspirin, various statins (e.g., pitavastatin, atorvastatin, lovastatin, pravastatin, rosuvastatin, simvastatin, promazine HCl, chloropromazine HCl, thioridazine HCl, Polymyxin B sulfate, chloroxine, benfluorex HCl and phenazopyridine HCl), and fluoxetine. Other therapeutic agents include antimicrobials (aminoclyclosides (e.g. gentamicin, neomycin, streptomycin), penicillins (e.g., amoxicillin, ampicillin), glycopeptides (e.g., avoparcin, vancomycin), macrolides (e.g., erythromycin, tilmicosin, tylosin), quinolones (e.g., sarafloxacin, enrofloxin), streptogramins (e.g., viginiamycin, quinupristin-dalfoprisitin), carbapenems, lipopeptides, oxazolidinones, cycloserine, ethambutol, ethionamide, isoniazrid, para-aminosalicyclic acid, and pyrazinamide). In some examples, an anti-viral (e.g., Abacavir, Aciclovir, Enfuvirtide, Entecavir, Nelfinavir, Nevirapine, Nexavir, Oseltamivir Raltegravir, Ritonavir, Stavudine, and Valaciclovir). The therapeutic may include a protein-based therapy for the treatment of various diseases, e.g., cancer, infectious diseases, hemophilia, anemia, multiple sclerosis, and hepatitis B or C.

Additional exemplary an additional payload can also include detectable markers or labels such as methylene blue, Patent blue V, and Indocyanine green.

The methods described herein may also include an additional payload may be added and may include a detectable moiety, or a detectable nanoparticle (e.g., a quantum dot). The detectable moiety may include a fluorescent molecule or a radioactive agent (e.g., ¹²⁵I). When the fluorescent molecule is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as ¹⁵²Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

In additional embodiments, the payload to be delivered may include a composition that edits genomic DNA (i.e., gene editing tools). For example, the gene editing composition may include a compound or complex that cleaves, nicks, splices, rearranges, translocates, recombines, or otherwise alters genomic DNA. Alternatively or in addition, a gene editing composition may include a compound that (i) may be included a gene-editing complex that cleaves, nicks, splices, rearranges, translocates, recombines, or otherwise alters genomic DNA; or (ii) may be processed or altered to be a compound that is included in a gene-editing complex that cleaves, nicks, splices, rearranges, translocates, recombines, or otherwise alters genomic DNA. In various embodiments, the gene editing composition comprises one or more of (a) gene editing protein; (b) RNA molecule; and/or (c) ribonucleoprotein (RNP).

In some embodiments, the gene editing composition comprises a gene editing protein, and the gene editing protein is a zinc finger nuclease (ZFN), a transcription activator-like effector nuclease (TALEN), a Cas protein, a Cre recombinase, a Hin recombinase, or a Flp recombinase. In additional embodiments, the gene editing protein may be a fusion proteins that combine homing endonucleases with the modular DNA binding domains of TALENs (megaTAL). For example, megaTAL may be delivered as a protein or alternatively, a mRNA encoding a megaTAL protein is delivered to the cells.

In various embodiments, the gene editing composition comprises a RNA molecule, and the RNA molecule comprises a sgRNA, a crRNA, and/or a tracrRNA.

In certain embodiments, the gene editing composition comprises a RNP, and the RNP comprises a Cas protein and a sgRNA or a crRNA and a tracrRNA. Aspects of the present subject matter are particularly useful for controlling when and for how long a particular gene-editing compound is present in a cell.

In various implementations of the present subject matter, the gene editing composition is detectable in a population of cells, or the progeny thereof, for (a) about 0.5, 1, 2, 3, 4, 5, 6, 7, 8, 9, 10, 12, 24, 48, 60, 72, 0.5-2, 0.5-6, 6-12 or 0.5-72 hours after the population of cells is contacted with the aqueous solution, or (b) less than about 0.5, 1, 2, 3, 4, 5, 6, 7, 8, 9, 10, 12, 24, 48, 60, 72, 0.5-2, 0.5-6, 6-12 or 0.5-72 hours after the population of cells is contacted with the aqueous solution.

In some embodiments, the genome of cells in the population of cells, or the progeny thereof, comprises at least one site-specific recombination site for the Cre recombinase, Hin recombinase, or Flp recombinase.

Aspects of the present invention relate to cells that comprise one gene editing compound, and inserting another gene editing compound into the cells. For example, one component of an RNP could be introduced into cells that express or otherwise already contain another component of the RNP. For example, cells in a population of cells, or the progeny thereof, may comprise a sgRNA, a crRNA, and/or a tracrRNA. In some embodiments the population of cells, or the progeny thereof, expresses the sgRNA, crRNA, and/or tracrRNA. Alternatively or in addition, cells in a population of cells, or the progeny thereof, express a Cas protein.

Various implementations of the subject matter herein include a Cas protein. In some embodiments, the Cas protein is a Cas9 protein or a mutant thereof. Exemplary Cas proteins (including Cas9 and non-limiting examples of Cas9 mutants) are described herein.

The Streptococcus pyogenes Cas9 NCBI Reference Sequence: NZ_CP010450.1 protein sequence is provided below (SEQ ID NO: 24)

MDKKYSIGLDIGTNSVGWAVITDDYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLADSTD
KADLRLIYLALAHMIKFRGHFLIEGDLNPDNSDVDKLFIQLVQTYNQLF
EENPINASRVDAKAILSARLSKSRRLENLIAQLPGEKKNGLFGNLIALL
LGLTPNFKSNFDLAEDAKLQLSKDTYDDDLDNLLAQIGDQYADLFLAAK
NLSDAILLSDILRVNSEITKAPLSASMIKRYDEHHQDLTLLKALVRQQL
PEKYKEIFFDQSKNGYAGYIDGGASQEEFYKFIKPILEKMDGTEELLAK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLPEDKEMIEERLK
KYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSDILKEYPVENTQLQNEKLYLYYLQNGRDMYVDQELDINRL
SDYDVDHIVPQSFLKDDSIDNKVLTRSDKNRGKSDNVPSEEVVKKMKNY
WKQLLNAKLITQRKFDNLTKAERGGLSELDKAGFIKRQLVETRQITKHV
AQILDSRMNTKYDENDKLIREVRVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSPEK
NPIDFLEAKGYKEVRKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

The Staphylococcus agnetis Cas9 NCBI Reference Sequence: NZ_CP045927.1 amino acid sequence is provided below (SEQ ID NO: 25)

MNNYILGLDIGITSVGYGIVDSDTREIKDAGVRLFPEANVDNNEGRRSK
RGARRLKRRRIHRLDRVKHLLAEYNLLDLTNIPKSTNPYQIRVKGLNEK
LSKDELVIALLHIAKRRGIHNVNVMMDDNDSGNELSTKDQLKKNAKALS
DKYVCELQLERFEQDYKVRGEKNRFKTEDFVREARKLLETQSKFFEIDQ
TFIMRYIDLVETRREYFEGPGKGSPFGWEGNIKKWFEQMMGHCTYFPEE
LRSVKYAYSAELFNALNDLNNLVITRDEEAKLNYGEKFQIIENVFKQKK
TPNLKQIAKEIGVSETDIKGYRVNKSGKPEFTQFKLYHDLKNIFEDSKY
LNDVQLMDNIAEIITIYQDPESIIKELNQLPELLSEKEKEKISALSGYA
GTHRLSLKCINLLLDDLWESSLNQMELFTKLNLKPKKIDLSQQHKIPIK
LVDDFILSPVVKRAFIQSIQVVNAIIDKYGLPEDIIIELARENNSDDRR
KFLNQLQKQNAETRKQVEKVLREYGNDNAKRIVQKIKLHNMQEGKCLYS
LKDIPLEDLLKNPNHYEVDHIIPRSVAFDNSMHNKVLVRAEENSKKGNR
TPYQYLNSSESSLSYNEFKQHILNLSKTKDRITKKKREYLLEERDINKY
DVQKEFINRNLVDTRYATRELTSLLKAYFSANNLDVKVKTINGSFTNYL
RKVWKFDKDRNKGYKHHAEDALIIANADFLFKHNKKLRNINKVLDAPSK
EVDKKRVTVQSEDEYNQMFEDTQKAQAIKKFEIRKFSHRVDKKPNRQLI
KDTLYSTRNIDGIEYVVESIKDIYSVNNDKVKTKFKKDPHRLLMYRNDP
QTFEKFEKVFKQYESEKNPFAKYYEETGEKIRKFSKTGQGPYINKIKYL
RERLGRHCDVTNKYINSRNKIVQLKIYSYRFDIYQYGNNYKMITISYID
LEQKSNYYYISREKYEQKKKDKQIDDSYKFIGSFYKNDIINYNGEMYRV
IGVNDSEKIKFSLI

The Synthetic construct derived from Staphylococcus aureus Cas9 NCBI Reference Sequence: MN548085.1 is provided below (SEQ ID NO:26)

MAPKKKRKVGIHGVPAAKRNYILGLDIGITSVGYGIIDYETRDVIDAGV
RLFKEANVENNEGRRSKRGARRLKRRRRHRIQRVKKLLFDYNLLTDHSE
LSGINPYEARVKGLSQKLSEEEFSAALLHLAKRRGVHNVNEVEEDTGNE
LSTREQISRNSKALEEKYVAELQLERLKKDGEVRGSINRFKTSDYVKEA
KQLLKVQKAYHQLDQSFIDTYIDLLETRRTYYEGPGEGSPFGWKDIKEW
YEMLMGHCTYFPEELRSVKYAYNADLYNALNDLNNLVITRDENEKLEYY
EKFQIIENVFKQKKKPTLKQIAKEILVNEEDIKGYRVTSTGKPEFTNLK
VYHDIKDITARKEIIENAELLDQIAKILTIYQSSEDIQEELTNLNSELT
QEEIEQISNLKGYTGTHNLSLKAINLILDELWHTNDNQIAIFNRLKLVP
KKVDLSQQKEIPTTLVDDFILSPVVKRSFIQSIKVINAIIKKYGLPNDI
IIELAREKNSKDAQKMINEMQKRNRQTNERIEEIIRTTGKENAKYLIEK
IKLHDMQEGKCLYSLEAIPLEDLLNNPFNYEVDHIIPRSVSFDNSFNNK
VLVKQEENSKKGNRTPFQYLSSSDSKISYETFKKHILNLAKGKGRISKT
KKEYLLEERDINRFSVQKDFINRNLVDTRYATRGLMNLLRSYFRVNNLD
VKVKSINGGFTSFLRRKWKFKKERNKGYKHHAEDALIIANADFIFKEWK
KLDKAKKVMENQMFEERQAESMPEIETEQEYKEIFITPHQIKHIKDFKD
YKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLT
KYSKKDNGPVIKKIKYYGNKLNAHLDITDDYPNSRNKVVKLSLKPYRFD
VYLDNGVYKFVTVKNLDVIKKENYYEVNSKCYEEAKKLKKISNQAEFIA
SFYNNDLIKINGELYRVIGVNNDLLNRIEVNMIDITYREYLENMNDKRP
PRIIKTIASKTQSIKKYSTDILGNLYEVKSKKHPQIIKKGKRPAATKKA
GQAKKKKGSYPYDVPDYASGFANELGPRLMGK

The Candidatus Methanomethylophilus alvus Mx1201 Cas12a NCBI Reference Sequence: NC_020913.1 (SEQ ID NO: 27) is provided below.

MHTGGLLSMDAKEFTGQYPLSKTLRFELRPIGRTWDNLEASGYLAEDRH
RAECYPRAKELLDDNHRAFLNRVLPQIDMDWHPIAEAFCKVHKNPGNKE
LAQDYNLQLSKRRKEISAYLQDADGYKGLFAKPALDEAMKIAKENGNES
DIEVLEAFNGFSVYFTGYHESRENIYSDEDMVSVAYRITEDNFPRFVSN
ALIFDKLNESHPDIISEVSGNLGVDDIGKYPDVSNYNNFLSQAGIDDYN
HIIGGHTTEDGLIQAFNVVLNLRHQKDPGPEKIQFKQLYKQILSVRTSK
SYIPKQFDNSKEMVDCICDYVSKIEKSETVERALKLVRNISSFDLRGIF
VNKKNLRILSNKLIGDWDAIETALMHSSSSENDKKSVYDSAEAFTLDDI
FSSVKKFSDASAEDIGNRAEDICRVISETAPFINDLRAVDLDSLNDDGY
EAAVSKIRESLEPYMDLFHELEIFSVGDEFPKCAAFYSELEEVSEQLIE
IIPLFNKARSFCTRKRYSTDKIKVNLKFPTLADGWDLNKERDNKAAILR
KDGKYYLAILDMKKDLSSIRTSDEDESSFEKMEYKLLPSPVKMLPKIFV
KSKAAKEKYGLTDRMLECYDKGMHKSGSAFDLGFCHELIDYYKRCIAEY
PGWDVFDFKFRETSDYGSMKEFNEDVAGAGYYMSLRKIPCSEVYRLLDE
KSIYLFQIYNKDYSENAHGNKNMHTMYWEGLFSPQNLESPVFKLSGGAE
LFPRKSSIPNDAKTVHPKGSVLVPRNDVNGRRIPDSIYRELTRYFNRGD
CRISDEAKSYLDKVKTKKADHDIVKDRRFTVDKMMFHVPIAMNFKAISK
PNLNKKVIDGIIDDQDLKIIGIDRGERNLIYVTMVDRKGNILYQDSLNI
LNGYDYRKALDVREYDNKEARRNWTKVEGIRKMKEGYLSLAVSKLADMI
IENNAIIVMEDLNHGFKAGRSKIEKQVYQKFESMLINKLGYMVLKDKSI
DQSGGALHGYQLANHVTTLASVGKQCGVIFYIPAAFTSKIDPTTGFADL
FALSNVKNVASMREFFSKMKSVIYDKAEGKFAFTFDYLDYNVKSECGRT
LWTVYTVGERFTYSRVNREYVRKVPTDIIYDALQKAGISVEGDLRDRIA
ESDGDTLKSIFYAFKYALDMRVENREEDYIQSPVKNASGEFFCSKNAGK
SLPQDSDANGAYNIALKGILQLRMLSEQYDPNAESIRLPLITNKAWLTF
MQSGMKTWKN

The Candidatus Methanomethylophilus alvus isolate MGYG-HGUT-02456 Cas12a NCBI Reference Sequence: NZ_LR699000.1 (SEQ ID NO: 28) is provided below:

MDAKEFTGQYPLSKTLRFELRPIGRTWDNLEASGYLAEDRHRAECYPRA
KELLDDNHRAFLNRVLPQIDMDWHPIAEAFCKVHKNPGNKELAQDYNLQ
LSKRRKEISAYLQDADGYKGLFAKPALDEAMKIAKENGNESDIEVLEAF
NGFSVYFTGYHESRENIYSDEDMVSVAYRITEDNFPRFVSNALIFDKLN
ESHPDIISEVSGNLGVDDIGKYFDVSNYNNFLSQAGIDDYNHIIGGHTT
EDGLIQAFNVVLNLRHQKDPGFEKIQFKQLYKQILSVRTSKSYIPKQFD
NSKEMVDCICDYVSKIEKSETVERALKLVRNISSFDLRGIFVNKKNLRI
LSNKLIGDWDAIETALMHSSSSENDKKSVYDSAEAFTLDDIFSSVKKFS
DASAEDIGNRAEDICRVISETAPFINDLRAVDLDSLNDDGYEAAVSKIR
ESLEPYMDLFHELEIFSVGDEFPKCAAFYSELEEVSEQLIEIIPLFNKA
RSFCTRKRYSTDKIKVNLKFPTLADGWDLNKERDNKAAILRKDGKYYLA
ILDMKKDLSSIRTSDEDESSFEKMEYKLLPSPVKMLPKIFVKSKAAKEK
YGLTDRMLECYDKGMHKSGSAFDLGFCHELIDYYKRCIAEYPGWDVFDF
KPRETSDYGSMKEFNEDVAGAGYYMSLRKIPCSEVYRLLDEKSIYLFQI
YNKDYSENAHGNKNMHTMYWEGLFSPQNLESPVFKLSGGAELFFRKSSI
PNDAKTVHPKGSVLVPRNDVNGRRIPDSIYRELTRYFNRGDCRISDEAK
SYLDKVKTKKADHDIVKDRRFTVDKMMFHVPIAMNFKAISKPNLNKKVI
DGIIDDQDLKIIGIDRGERNLIYVTMVDRKGNILYQDSLNILNGYDYRK
ALDVREYDNKEARRNWTKVEGIRKMKEGYLSLAVSKLADMIIENNAIIV
MEDLNHGFKAGRSKIEKQVYQKFESMLINKLGYMVLKDKSIDQSGGALH
GYQLANHVTTLASVGKQCGVIFYIPAAFTSKIDPTTGFADLFALSNVKN
VASMREFFSKMKSVIYDKAEGKFAFTFDYLDYNVKSECGRTLWTVYTVG
ERFTYSRVNREYVRKVPTDIIYDALQKAGISVEGDLRDRIAESDGDTLK
SIFYAFKYALDMRVENREEDYIQSPVKNASGEFFCSKNAGKSLPQDSDA
NGAYNIALKGILQLRMLSEQYDPNAESIRLPLITNKAWLTFMQSGMKTW
KN

The Candidatus Methanoplasma termitum strain MpT1 chromosome Cas12a NCBI Reference Sequence: NZ_CP010070.1 (SEQ ID NO: 29) is provided below:

MNNYDEFTKLYPIQKTIRFELKPQGRTMEHLETFNFFEEDRDRAEKYKI
LKEAIDEYHKKFIDEHLTNMSLDWNSLKQISEKYYKSREEKDKKVFLSE
QKRMRQEIVSEFKKDDRFKDLFSKKLFSELLKEEIYKKGNHQEIDALKS
PDKFSGYFIGLHENRKNMYSDGDEITAISNRIVNENFPKFLDNLQKYQE
ARKKYPEWIIKAESALVAHNIKMDEVFSLEYFNKVLNQEGIQRYNLALG
GYVTKSGEKMMGLNDALNLAHQSEKSSKGRIHMTPLFKQILSEKESFSY
IPDVFTEDSQLLPSIGGFFAQIENDKDGNIFDRALELISSYAEYDTERI
YIRQADINRVSNVIFGEWGTLGGLMREYKADSINDINLERTCKKVDKWL
DSKEFALSDVLEAIKRTGNNDAFNEYISKMRTAREKIDAARKEMKFISE
KISGDEESIHIIKTLLDSVQQFLHFFNLFKARQDIPLDGAFYAEFDEVH
SKLFAIVPLYNKVRNYLTKNNLNTKKIKLNFKNPTLANGWDQNKVYDYA
SLIFLRDGNYYLGIINPKRKKNIKFEQGSGNGPFYRKMVYKQIPGPNKN
LPRVFLTSTKGKKEYKPSKEIIEGYEADKHIRGDKFDLDFCHKLIDFFK
ESIEKHKDWSKFNFYFSPTESYGDISEFYLDVEKQGYRMHFENISAETI
DEYVEKGDLFLFQIYNKDFVKAATGKKDMHTIYWNAAFSPENLQDVVVK
LNGEAELFYRDKSDIKEIVHREGEILVNRTYNGRTPVPDKIHKKLTDYH
NGRTKDLGEAKEYLDKVRYFKAHYDITKDRRYLNDKIYFHVPLTLNFKA
NGKKNLNKMVIEKFLSDEKAHIIGIDRGERNLLYYSIIDRSGKIIDQQS
LNVIDGPDYREKLNQREIEMKDARQSWNAIGKIKDLKEGYLSKAVHEIT
KMAIQYNAIVVMEELNYGFKRGRFKVEKQIYQKFENMLIDKMNYLVFKD
APDESPGGVLNAYQLTNPLESFAKLGKQTGILFYVPAAYTSKIDPTTGF
VNLFNTSSKTNAQERKEFLQKFESISYSAKDGGIFAFAFDYRKFGTSKT
DHKNVWTAYTNGERMRYIKEKKRNELFDPSKEIKEALTSSGIKYDGGQN
ILPDILRSNNNGLIYTMYSSFIAAIQMRVYDGKEDYIISPIKNSKGEFF
RTDPKRRELPIDADANGAYNIALRGELTMRAIAEKFDPDSEKMAKLELK
HKDWFEFMQTRGD

In certain embodiments, the gene editing composition comprises (a) a first sgRNA molecule and a second sgRNA molecule, wherein the nucleic acid sequence of the first sgRNA molecule is different from the nucleic acid sequence of the second sgRNA molecule; (b) a first RNP comprising a first sgRNA and a second RNP comprising a second sgRNA, wherein the nucleic acid sequence of the first sgRNA molecule is different from the nucleic acid sequence of the second sgRNA molecule; (c) a first crRNA molecule and a second crRNA molecule, wherein the nucleic acid sequence of the first crRNA molecule is different from the nucleic acid sequence of the second crRNA molecule; (d) a first crRNA molecule and a second crRNA molecule, wherein the nucleic acid sequence of the first crRNA molecule is different from the nucleic acid sequence of the second crRNA molecule, and further comprising a tracrRNA molecule; or (e) a first RNP comprising a first crRNA and a tracrRNA and a second RNP comprising a second crRNA and a tracrRNA, wherein the nucleic acid sequence of the first crRNA molecule is different from the nucleic acid sequence of the second crRNA molecule.

In aspects, the ratio of the Cas9 protein to guide RNA may be 1:1, 1:2, 1:3, 1:4, 1:5, 1:6, 1:7, 1:8, 1:9, or 1:10.

In embodiments, increasing the number of times that cells go through the delivery process (alternatively, increasing the number of doses), may increase the percentage edit; wherein, in some embodiments the number of doses may include 1, 2, 3, 4, 5, 6, 7, 8, 9, or 10 doses.

In various embodiments, the first and second sgRNA or first and second crRNA molecules together comprise nucleic acid sequences complementary to target sequences flanking a gene, an exon, an intron, an extrachromosomal sequence, or a genomic nucleic acid sequence, wherein the gene, an exon, intron, extrachromosomal sequence, or genomic nucleic acid sequence is about 1, 2, 3, 4, 5, 6, 10, 20, 30, 40, 50, 60, 70, 80, 90, 100, 1-100, kilobases in length or is at least about 1, 2, 3, 4, 5, 6, 10, 20, 30, 40, 50, 60, 70, 80, 90, 100, 1-100, kilobases in length. In some embodiments, the use of pairs of RNPs comprising the first and second sgRNA or first and second crRNA molecules may be used to create a polynucleotide molecule comprising the gene, exon, intron, extrachromosomal sequence, or genomic nucleic acid sequence.

In certain embodiments, the target sequence of a sgRNA or crRNA is about 12 to about 25, or about 12, 13, 14, 15, 16, 17, 18, 19, 20, 21, 22, 23, 24, 25, 17-23, or 18-22, nucleotides long. In some embodiments, the target sequence is 20 nucleotides long or about 20 nucleotides long.

In various embodiments, the first and second sgRNA or first and second crRNA molecules are complementary to sequences flanking an extrachromosomal sequence that is within an expression vector.

Aspects of the present subject matter relate to the delivery of multiple components of a gene-editing complex, where the multiple components are not complexed together. In some embodiments, gene editing composition comprises at least one gene editing protein and at least one nucleic acid, wherein the gene editing protein and the nucleic acid are not bound to or complexed with each other.

The present subject matter allows for high gene editing efficiency while maintaining high cell viability. In some embodiments, at least about 10, 20, 30, 40, 50, 60, 70, 80, 90, 95, 99%, 1-99%, or more of the population of cells, or the progeny thereof, become genetically modified after contact with the aqueous solution. In various embodiments, at least about 10, 20, 30, 40, 50, 60, 70, 80, 90, 95, 99%, 1-99%, or more of the population of cells, or the progeny thereof, are viable after contact with the aqueous solution.

In certain embodiments, the gene editing composition induces single-strand or double-strand breaks in DNA within the cells. In some embodiments the gene editing composition further comprises a repair template polynucleotide. In various embodiments, the repair template comprises (a) a first flanking region comprising nucleotides in a sequence complementary to about 40 to about 90 base pairs on one side of the single or double strand break and a second flanking region comprising nucleotides in a sequence complementary to about 40 to about 90 base pairs on the other side of the single or double strand break; or (b) a first flanking region comprising nucleotides in a sequence complementary to at least about 20, 25, 30, 35, 40, 45, 50, 60, 70, 80, or 90 base pairs on one side of the single or double strand break and a second flanking region comprising nucleotides in a sequence complementary to at least about 20, 25, 30, 35, 40, 45, 50, 60, 70, 80, or 90 base pairs on the other side of the single or double strand break. Non-limiting descriptions relating to gene editing (including repair templates) using the CRISPR-Cas system are discussed in Ran et al. (2013) Nat Protoc. 2013 November; 8(11): 2281-2308, the entire content of which is incorporated herein by reference. Embodiments involving repair templates are not limited to those comprising the CRISPR-Cas system.

In various implementations of the present subject matter, the volume of aqueous solution is delivered to the population of cells in the form of a spray. In some embodiments, the volume is between 6.0×10⁻⁷ microliter per cell and 7.4×10⁻⁴ microliter per cell. In certain embodiments, the spray comprises a colloidal or sub-particle comprising a diameter of 10 nm to 100 μm. In various embodiments, the volume is between 2.6×10⁻⁹ microliter per square micrometer of exposed surface area and 1.1×10⁻⁶ microliter per square micrometer of exposed surface area.

In some embodiments, the RNP has a size of approximately 100 Å×100 Å×50 Å or 10 nm×10 nm×5 nm. In various embodiments, the size of spray particles is adjusted to accommodate at least about 1, 2, 3, 4, 5, 6, 7, 8, 9, 10, or more RNPs per spray particle.

For example, contacting the population of cells with the volume of aqueous solution may be performed by gas propelling the aqueous solution to form a spray. In certain embodiments, the population of cells is in contact with said aqueous solution for 0.01-10 minutes (e.g., 0.1 10 minutes) prior to adding a second volume of buffer or culture medium to submerse or suspend said population of cells.

In various embodiments, the population of cells includes at least one of primary or immortalized cells. For example, the population of cells may include mesenchymal stem cells, lung cells, neuronal cells, fibroblasts, human umbilical vein (HUVEC) cells, and human embryonic kidney (HEK) cells, primary or immortalized hematopoietic stem cell (HSC), T cells, natural killer (NK) cells, cytokine-induced killer (CIK) cells, human cord blood CD34+ cells, B cells. Non limiting examples of T cells may include CD8+ or CD4+ T cells. In some aspects, the CD8+ subpopulation of the CD3⁺ T cells are used. CD8⁺ T cells may be purified from the PBMC population by positive isolation using anti-CD8 beads. In some aspects primary NK cells are isolated from PBMCs and GFP mRNA may be delivered by platform delivery technology (i.e., 3% expression and 96% viability at 24 hours). In additional aspects, NK cell lines, e.g., NK92 may be used.

Cell types also include cells that have previously been modified for example T cells, NK cells and MSC to enhance their therapeutic efficacy. For example: T cells or NK cells that express chimeric antigen receptors (CAR T cells, CAR NK cells, respectively); T cells that express modified T cell receptor (TCR); MSC that are modified virally or non-virally to overexpress therapeutic proteins that complement their innate properties (e.g. delivery of Epo using lentiviral vectors or BMP-2 using AAV-6) (reviewed in Park et al, Methods, 2015 August; 84-16.); MSC that are primed with non-peptidic drugs or magnetic nanoparticles for enhanced efficacy and externally regulated targeting respectively (Park et al., 2015); MSC that are functionalised with targeting moieties to augment their homing toward therapeutic sites using enzymatic modification (e.g. Fucosyltransferase), chemical conjugation (eg. modification of SLeX on MSC by using N-hydroxy-succinimide (NHS) chemistry) or non-covalent interactions (eg. engineering the cell surface with palmitated proteins which act as hydrophobic anchors for subsequent conjugation of antibodies) (Park et al., 2015). For example, T cells, e.g., primary T cells or T cell lines, that have been modified to express chimeric antigen receptors (CAR T cells) may further be treated according to the invention with gene editing proteins and or complexes containing guide nucleic acids specific for the CAR encoding sequences for the purpose of editing the gene(s) encoding the CAR, thereby reducing or stopping the expression of the CAR in the modified T cells.

Aspects of the present invention relate to the expression vector-free delivery of gene editing compounds and complexes to cells and tissues, such as delivery of Cas-gRNA ribonucleoproteins for genome editing in primary human T cells, hematopoietic stem cells (HSC), and mesenchymal stromal cells (MSC). In some example, mRNA encoding such proteins are delivered to the cells.

Various aspects of the CRISPR-Cas system are known in the art. Non-limiting aspects of this system are described, e.g., in U.S. Pat. No. 9,023,649, issued May 5, 2015; U.S. Pat. No. 9,074,199, issued Jul. 7, 2015; U.S. Pat. No. 8,697,359, issued Apr. 15, 2014; U.S. Pat. No. 8,932,814, issued Jan. 13, 2015; PCT International Patent Application Publication No. WO 2015/071474, published Aug. 27, 2015; Cho et al., (2013) Nature Biotechnology Vol 31 No 3 pp 230-232 (including supplementary information); and Jinek et al., (2012) Science Vol 337 No 6096 pp 816-821, the entire contents of each of which are incorporated herein by reference.

In one aspect, the present subject matter describes cells attached to a solid support, (e.g., a strip, a polymer, a bead, or a nanoparticle). The support or scaffold may be a porous or non-porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present subject matter. The support material may have virtually any possible structural configuration. Thus, the support configuration may be spherical, as in a bead, or cylindrical, as in the inside surface of a test tube, or the external surface of a rod. Alternatively, the surface may be flat such as a sheet, or test strip, etc. Preferred supports include polystyrene beads.

In other aspects, the solid support comprises a polymer, to which cells are chemically bound, immobilized, dispersed, or associated. A polymer support may be a network of polymers, and may be prepared in bead form (e.g., by suspension polymerization). The cells on such a scaffold can be sprayed with payload containing aqueous solution according to the invention to deliver desired compounds to the cytoplasm of the scaffold. Exemplary scaffolds include stents and other implantable medical devices or structures.

The present subject matter further relates to apparatus, systems, techniques and articles for delivery of payloads across a plasma membrane. The present subject matter also relates to an apparatus for delivering payloads such as proteins or protein complexes across a plasma membrane (coronavirus antigens, coronavirus mRNA molecules, coronavirus synthetic mRNAs, or DNA-encoding coronavirus antigens peptides). The current subject matter may find utility in the field of intra-cellular delivery, and has application in, for example, delivery of molecular biological and pharmacological therapeutic agents to a target site, such as a cell, tissue, or organ.

In some implementations, an apparatus for delivering a payload across a plasma membrane can include an atomizer having at least one atomizer emitter and a support oriented relative to the atomizer. The method further comprises the step of atomizing the payload prior to contacting the plasma membrane with the payload.

The atomizer can be selected from a mechanical atomizer, an ultrasonic atomizer, an electrospray, a nebuliser, and a Venturi tube. The atomizer can be a commercially available atomizer. The atomizer can be an intranasal mucosal atomization device. The atomizer can be an intranasal mucosal atomization device commercially available from LMA Teleflex of NC, USA. The atomizer can be an intranasal mucosal atomization device commercially available from LMA Teleflex of NC, USA under catalogue number MAD300.

The atomizer can be adapted to provide a colloid suspension of particles having a diameter of 30-100 μm prior to contacting the plasma membrane with the payload. The atomizer can be adapted to provide a colloid suspension of particles having a diameter of 30-80 μm. The atomizer can be adapted to provide a colloid suspension of particles having a diameter of 50-80 μm.

The atomizer can include a gas reservoir. The atomizer can include a gas reservoir with the gas maintained under pressure. The gas can be selected from air, carbon dioxide, and helium. The gas reservoir can include a fixed pressure head generator. The gas reservoir can be in fluid communication with the atomizer emitter. The gas reservoir can include a gas guide, which can be in fluid communication with the atomizer emitter. The gas guide can be adapted to allow the passage of gas therethrough. The gas guide can include a hollow body. The gas guide can be a hollow body having open ends. The gas guide can include a hollow body having first and second open ends. The gas guide can be a hollow body having first and second opposing open ends. The diameter of the first open end can be different to the diameter of the second open end. The diameter of the first open end can be different to the diameter of the second open end. The diameter of the first open end can be greater than the diameter of the second open end. The first open end can be in fluid communication with the gas reservoir. The second open end can be in fluid communication with the atomizer emitter.

The apparatus can include a sample reservoir. The sample reservoir can be in fluid communication with the atomizer. The sample reservoir can be in fluid communication with the atomizer emitter. The gas reservoir and the sample reservoir can both be in fluid communication with the atomizer emitter.

The apparatus can include a sample valve located between the sample reservoir and the gas reservoir. The apparatus can include a sample valve located between the sample reservoir and the gas guide. The sample valve can be adapted to adjust the sample flow from the sample reservoir. The sample valve can be adapted to allow continuous or semi-continuous sample flow. The sample valve can be adapted to allow semi-continuous sample flow. The sample valve can be adapted to allow semi-continuous sample flow of a defined amount. The sample valve is adapted to allow semi-continuous sample flow of 0.5-100 μL. The sample valve can be adapted to allow semi-continuous sample flow of 10 μL. The sample valve can be adapted to allow semi-continuous sample flow of 1 μL to an area of 0.065-0.085 cm².

The atomizer and the support can be spaced apart. The support can include a solid support. The support can include a plate including sample wells. The support can include a plate including sample wells selected from 1, 6, 9, 12, 24, 48, 384, 1536 or more wells. Alternatively, the support comprises a plate, e.g., a scaled up configuration that can accommodate a monolayer with more cells than a microtiter plate. The solid support can be formed from an inert material. The solid support can be formed from a plastic material, or a metal or metal alloy, or a combination thereof. The support can include a heating element. The support can include a resistive element. The support can be reciprocally mountable to the apparatus. The support can be reciprocally movable relative to the apparatus. The support can be reciprocally movable relative to the atomizer. The support can be reciprocally movable relative to the atomizer emitter. The support can include a support actuator to reciprocally move the support relative to the atomizer. The support can include a support actuator to reciprocally move the support relative to the atomizer emitter. The support can include a support actuator to reciprocally move the support relative to the longitudinal axis of the atomizer emitter. The support can include a support actuator to reciprocally move the support transverse to the longitudinal axis of the atomizer emitter.

The longitudinal axis of the spray zone can be coaxial with the longitudinal axis or center point of the support and/or the circular well of the support, to which the payload is to be delivered. The longitudinal axis of the atomizer emitter can be coaxial with the longitudinal axis or center point of the support and/or the circular well of the support. The longitudinal axis of the atomizer emitter, the longitudinal axis of the support, and the longitudinal axis of the spray zone can be each coaxial. The longitudinal length of the spray zone may be greater than the diameter (may be greater than double) of the circular base of the spray zone (e.g., the area of cells to which the payload is to be delivered).

The apparatus can include a valve located between the gas reservoir and the atomizer. The valve can be an electromagnetically operated valve. The valve can be a solenoid valve. The valve can be a pneumatic valve. The valve can be located at the gas guide. The valve can be adapted to adjust the gas flow within the gas guide. The valve can be adapted to allow continuous or semi-continuous gas flow. The valve can be adapted to allow semi-continuous gas flow. The valve can be adapted to allow semi-continuous gas flow of a defined time interval. The valve can be adapted to allow semi-continuous gas flow of a one second time interval. The apparatus can include at least one filter. The filter can include a pore size of less than 10 μm. The filter can have a pore size of 10 μm. The filter can be located at the gas guide. The filter can be in fluid communication with the gas guide.

The apparatus can include at least one regulator. The regulator can be an electrical regulator. The regulator can be a mechanical regulator. The regulator can be located at the gas guide. The regulator can be in fluid communication with the gas guide. The regulator can be a regulating valve. The pressure within the gas guide can be 1.0-2.0 bar. The pressure within the gas guide can be 1.5 bar. The pressure within the gas guide can be 1.0-2.0 bar, and the distance between the atomizer and the support can be less than or equal to 31 mm. The pressure within the gas guide can be 1.5 bar, and the distance between the atomizer and the support can be 31 mm. The pressure within the gas guide can be 0.05 bar per millimeter distance between the atomizer and the support. The regulating valve can be adapted to adjust the pressure within the gas guide to 1.0-2.0 bar. The regulating valve can be adapted to adjust the pressure within the gas guide to 1.5 bar. Each regulating valve can be adapted to maintain the pressure within the gas guide at 1.0-2.0 bar. Each regulating valve can be adapted to maintain the pressure within the gas guide at 1.5 bar.

The apparatus can include two regulators. The apparatus can include first and second regulators. The first and second regulator can be located at the gas guide. The first and second regulator can be in fluid communication with the gas guide. The first regulator can be located between the gas reservoir and the filter. The first regulator can be adapted to adjust the pressure from the gas reservoir within the gas guide to 2.0 bar. The first regulator can be adapted to maintain the pressure within the gas guide at 2.0 bar. The second regulator can be located between the filter and the valve.

The atomizer emitter can be adapted to provide a conical spray zone (e.g., a generally circular conical spray zone). The atomizer emitter can be adapted to provide a 30° conical spray zone. The apparatus further can include a microprocessor to control any or all parts of the apparatus. The microprocessor can be arranged to control any or all of the sample valve, the support actuator, the valve, and the regulator. The apparatus can include an atomizer having at least one atomizer emitter; and a support oriented relative to the atomizer; the atomizer can be selected from a mechanical atomizer, an ultrasonic atomizer, an electrospray, a nebuliser, and a Venturi tube. The atomizer can be adapted to provide a colloid suspension of particles having a diameter of 30-100 μm. The apparatus can include a sample reservoir and a gas guide, and a sample valve located between the sample reservoir and the gas guide. The sample valve can be adapted to allow semi-continuous sample flow of 10-100 μL. The atomizer and the support can be spaced apart and define a generally conical spray zone there between; and the distance between the atomizer and the support can be approximately double the diameter of the circular base of the area of cells to which molecules are to be delivered; the distance between the atomizer and the support can be 31 mm and the diameter of the circular base of the area of cells to which molecules are to be delivered can be 15.5 mm. The apparatus can include a gas guide and the pressure within the gas guide is 1.0-2.0 bar. The apparatus can include at least one filter having a pore size of less than 10 μm.

The aqueous solution and/or composition can be saponin-free.

The details of one or more variations of the subject matter described herein are set forth in the accompanying drawings and the description below. Other features and advantages of the subject matter described herein will be apparent from the description and drawings, and from the claims.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Delivery to DC for Epitope Presentation

In these studies, the SOLUPORE™ technology is used to deliver SARS-CoV-2-related molecules to dendritic cells (DCs). Epitope presentation and T cell activation are examined Exemplary SARS-CoV-2 related molecules include DNA, mRNA or protein, in particular for 1) full length Spike(S) protein (SEQ ID NO: 1), 2) spike protein subunit 2 (S2) (SEQ ID NO: 4), 3) spike protein subunit 1 (S1) (SEQ ID NO: 3), 4) D614G variant (of SEQ ID NO: 1), and 5) variants including K417N, K417T, N439K, L452R, Y453F, S477N, E484K, N501Y, D253G, L18F, R246I, L452R, P681H, A701V, Q677P, and/or Q677H of SEQ ID NO: 1.

In addition, TriMix mRNAs (e.g., mRNAs encoding CD40L, caTLR4 and/or CD70) are co-delivered with the SARS-CoV-2 related molecules to determine whether responses, such as epitope presentation or T cell activation would be enhanced.

DC are loaded with 0.1 mg, 0.33 mg or 1.0 mg SARS-CoV-2 spike protein, with or without GM-CSF. In particular, full length spike protein (SEQ ID NO: 1) is loaded to DCs. In other examples, fragments of spike protein (SEQ ID NO: 1) are loaded, including the 51 subunit (SEQ ID NO: 3) or the S2 subunit (SEQ ID NO: 4). In further examples, mutations or variants of the 51 protein are loaded to DCs, including for example, K417N, E484K, N501Y, K417T, E484K, and N501Y of SEQ ID NO: 1. In further examples, various combinations of spike protein fragments and/or mutations (or variants) are co-delivered to DCs. For example, full length spike protein (SEQ ID NO: 1), K417N, E484K, N501Y, K417T, E484K, and/or N501Y are co-delivered to DCs. In examples, any combination of variants can be delivered to DCs, for example, one variant, two variants, 3 variants, 4 variants, 5 variants, or 6 variants may be delivered to DCs. A mutation at the DNA level results in the variant virus, thus the payload (cargo) delivered to the DCs are variants.

DC antigen presentation is analysed in vitro whereby DCs are co-cultured with naïve CD4+ cells in vitro, for 14 d and re-stimulated with spike protein for 7 h. An increase in the percentage of CD4+CD154+IFNγ+ cells is observed indicating that DCs are successfully presenting spike protein antigens and inducing T cell responses. Similar responses are observed when DC are loaded with mRNA encoding for SARS-CoV-2 spike protein. TriMix mRNAs are co-delivered with either SARS-CoV-2 spike protein or with mRNA encoding for SARS-CoV-2 spike protein. A further increase in the percentage of CD4+CD154+IFNγ+ cells is observed. For example, a clinically relevant increase of CD4+CD154+IFNγ+ cells may be about 10-20%, about 10%, about 15%, or about 20% increase (e.g., relative to a control of non-genetically engineered DCs).

The components of the delivery solution (for delivery of payloads to DCs) includes 32.5 mM sucrose, 106 mM potassium chloride, 5 mM Hepes in water with a range of ethanol from about 2-50%, for example about 12% ethanol.

### Example 2: Engineering DCs to Enhance Functionality

DCs are engineered to enhance functionality (e.g., antigen presentation and/or activation of coronavirus-specific T cells), wherein an increased release of IFN gamma, IL-2, IL-8, IL-10 and/or TNF alpha is observed.

mRNAs encoding for IL-12, CXCL9 or the SNARE protein SEC22B are delivered simultaneously or sequentially with mRNA encoding for spike protein or spike protein itself. DC antigen presentation is analysed in vitro whereby DC were co-cultured with naïve CD4+ cells in vitro, for 14 d and re-stimulated with spike protein for 7 h. An increase in the percentage of CD4+CD154+IFNγ+ cells is observed in cells where IL-12, CXCL9 or the SNARE protein SEC22B is delivered indicating that they enhanced the ability of DC to induce T cell responses.

CRISPR Cas9 RNPs targeting PD-L1 and PD-L2 are delivered to DCs followed by delivery of mRNA encoding for spike protein or spike protein itself. DC antigen presentation is analysed in vitro whereby DC were co-cultured with naïve CD4+ cells in vitro, for 14 d and re-stimulated with spike protein for 7 h. An increase in the percentage of CD4+CD154+IFNγ+ cells is observed in cells where PD-L1 and PD-L2 were knocked down indicating that they enhance the ability of DC to induce T cell responses. For example, a clinically relevant increase of CD4+CD154+IFNγ+ cells may be about 10-20%, about 10%, about 15%, or about 20% increase (e.g., relative to a control of non-genetically engineered DCs).

### Example 3: Delivery of Allogenic DC

Allogeneic DCs are generated by maturing DC generated through differentiation and maturation of the AML cell line DCOne (available from DCPrime at dcprime.com/dcprime-obtains-patent-protection-for-dcone-platform/). The SOLUPORE™ technology is used to deliver SARS-CoV-2-related molecules to these DCs, and epitope presentation and T cell activation are examined. In addition, TriMix mRNAs are co-delivered with the SARS-CoV-2 related molecules, to determine whether the responses, such as epitope presentation and T cell activation are enhanced. The cells are cultured in a cocktail of Granulocyte-macrophage colony-stimulating factor (GM-CSF), TNFα, and IL-4 in the presence of mitoxantrone to accelerate DC differentiation, followed by maturation in the presence of prostaglandin-E2, TNFα, and IL-1β.

DC are loaded with 0.1 mg, 0.33 mg or 1.0 mg SARS-CoV-2 spike protein, with or without GM-CSF. DC antigen presentation is analysed in vitro whereby DC were co-cultured with naïve CD4+ cells in vitro, for 14 d and re-stimulated with spike protein for 7 h. An increase in the percentage of CD4+CD154+IFNγ+ cells is observed indicating that DC are successfully presenting spike protein antigens and inducing T cell responses. Similar responses are observed when DC are loaded with mRNA encoding for SARS-CoV-2 spike protein. TriMix mRNAs are co-delivered with either SARS-CoV-2 spike protein or with mRNA encoding for SARS-CoV-2 spike protein. A further increase in the percentage of CD4+CD154+IFNγ+ cells is observed. For example, a clinically relevant increase of CD4+CD154+IFNγ+ cells may be about 10-20%, about 10%, about 15%, or about 20% increase (e.g., relative to a control of non-genetically engineered DCs).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed:

1. A method for engineering dendritic cells (DCs) to present a payload comprising coronavirus antigens, coronavirus mRNA molecules, coronavirus synthetic mRNAs, or DNA-encoding coronavirus antigens peptides, comprising,
providing a population of DCs; and
contacting the population of cells with a volume of an isotonic aqueous solution, the aqueous solution including the payload and an alcohol at greater than 2 percent (v/v) concentration.

2. The method of claim 1, wherein the DCs are contacted with a mRNA encoding a protein comprising an amino acid sequence with at least 90% sequence identity to the amino acid sequence of SEQ ID NO: 30.

3. The method of claim 1, wherein the DCs are contacted with a mRNA encoding a protein comprising the amino acid sequence of SEQ ID NO: 30.

4. The method of claim 3, wherein the mRNA comprises the ribonucleic acid sequence of SEQ ID NO: 32.

5. The method of claim 1, wherein the payload is delivered to autologous cells ex vivo.

6. The method of claim 1, wherein the payload is delivered to allogenic cells ex vivo.

7. The method of claim 1, wherein the cells comprise DCOne cells or MUTZ-3 cells.

8. The method of claim 1, wherein the payload further comprises a DNA or mRNA encoding a Soluble N-ethylmaleimide-sensitive factor (NSF) attachment protein (SNAP) Receptor (SNARE) protein, wherein the SNARE protein comprises vesicle-trafficking protein SEC22B (SEC22B), interleukin 12 (IL-12), Chemokine (C-X-C motif) ligand 9 (CXCL9), or cluster of differentiation 40 (CD40L).

9. The method of claim 1, wherein the payload further comprises a DNA or mRNA encoding YTH N6-Methyladenosine RNA Binding Protein 1 (YTHDF1), gene editing proteins, programmed death ligand 1 (PD-L1), or programmed death ligand 2 (PD-L2).

10. A method of generating dendritic cell vaccines for infectious and non-infectious diseases according to claim 1.

11. A dendritic cell vaccine comprising mRNA encoding a coronavirus antigen delivered to autologous or allogenic dendritic cells.

12. The method of claim 1, wherein the alcohol comprises ethanol at a concentration from about 2-20% (v/v).

13. The method of claim 12, wherein the alcohol comprises ethanol at a concentration of about 12% (v/v).

14. The method of claim 1, wherein the aqueous solution comprises potassium chloride (KCl) comprises a concentration between 12.5-500 mM.

15. The method of claim 14, wherein the KCl comprises a concentration of 106 mM.

16. The method of claim 1, wherein the payload comprises mRNA encoding for severe acute respiratory syndrome coronavirus 2 (SARS-CoV-2) spike protein (SEQ ID NO: 1), or a fragment thereof.

17. The method of claim 1, wherein the payload comprises mRNA encoding for a SARS-CoV-2 spike protein variant.

18. The method of claim 14, wherein the spike protein variant comprises K417N, E484K, N501Y, K417T, E484K, and/or N501Y of SEQ ID NO: 1.

19. The method of claim 1, wherein the payload further comprises mRNA encoding for at least one of cluster of differentiation 40 ligand (CD40), constitutively active toll-like receptor 4 (caTLR4), and/or cluster of differentiation 70 (CD70).

20. The method of claim 1, wherein the payload further comprises Snap Receptor Protein (SNARE) protein, wherein the SNARE protein comprises vesicle-trafficking protein SEC22B (SEC22B).

21. The method of claim 20, wherein the payload comprises DNA or mRNA encoding SNARE and/or SEC22b.

22. The method of claim 1, wherein the engineered DCs have enhanced functionality and T cell response compared to control DCs, wherein the control DCs do not comprise a payload.

23. A dendritic cell comprising a protein comprising an amino acid sequence with at least 90% sequence identity to the amino acid sequence of SEQ ID NO: 30.

24. The dendritic cell of claim 23, wherein said dendritic cell comprises a protein comprising the amino acid sequence of SEQ ID NO: 30.
